(19) United States
(12) Patent Application Publication
(10) Pub. No.: US 2025/0154131 A1
(43) Pub. Date: May 15, 2025

(54) PYRROLIDINE ANTIVIRAL COMPOUND

(71) Applicant: HAINAN SIMCERE PHARMACEUTICAL CO., LTD., Haikou, Hainan (CN)

(72) Inventors: Hao ZOU, Shanghai (CN); Wei ZHU, Shanghai (CN); Lang LIU, Shanghai (CN); Weikun WANG, Shanghai (CN); Shun XU, Shanghai (CN); Zhengtao LI, Shanghai (CN)

(21) Appl. No.: 18/868,693

(22) PCT Filed: Nov. 25, 2022
(86) PCT No.: PCT/CN2022/134262

§ 371 Date: Jan. 16, 2025

(30) Foreign Application Priority Data
Nov. 26, 2021 (CN) 202111424968.3

(51) Int. Cl. C07D 403/12; A61K 31/404; A61K 31/437; A61K 31/454; A61P 31/14; C07D 401/14; C07D 471/04

(52) U.S. Cl. CPC C07D 403/12; A61K 31/404; A61K 31/437; A61K 31/454; A61P 31/14; C07D 401/14; C07D 471/04

(57) ABSTRACT

Provided are a pyrrolidine compound as represented by formula (I) and a pharmaceutically acceptable salt thereof, and the use thereof as a coronavirus 3CL protease inhibitor in the prevention or treatment of related diseases caused by coronavirus infections.

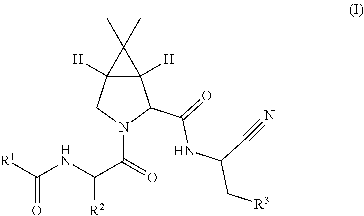

The present disclosure claims priority to the Chinese Patent Application No. 202111424968.3 filed with China National Intellectual Property Administration on Nov. 26, 2021, and entitled “PYRROLIDINE ANTIVIRAL COMPOUND”. The prior application described above is incorporated herein by reference in its entirety.

### TECHNICAL FIELD

The present disclosure relates to the field of medicines, and particularly to a novel pyrrolidine compound and a pharmaceutically acceptable salt thereof, and use thereof as a coronavirus 3CL protease inhibitor in the prevention or treatment of a related disease caused by coronavirus infection.

### BACKGROUND

Coronaviruses are a class of RNA viruses that can cause diseases in mammals and birds, leading to respiratory tract infections ranging from mild to severe and even fatal. The epidemics of diseases such as SARS, MERS, and SARS-CoV-2 (COVID-19) are all caused by coronaviruses. The SARS-CoV-2 (COVID-19) coronavirus contains four non-structural proteins: papain-like protease (PLPro), 3C-like protease (3CLPro), RNA polymerase, and helicase. Among them, 3CLpro is capable of hydrolyzing the viral polyproteins pp1a and pp1ab during the replication of the coronavirus to produce functional proteins. Thus, the 3C-like protease is essential for the replication of the virus. Currently, multiple preclinical and clinical studies have shown that inhibition of the 3CL protease can effectively suppress viral replication and reduce intracellular and in-vivo viral titers. However, to date, no 3CL protease inhibitors have been approved for clinical use. Given that the severe pneumonia caused by coronaviruses seriously affects people's life health, there is an urgent clinical need for effective small-molecule antiviral drugs, so that the research and development of 3CL protease inhibitors with novel structure, low toxicity and high efficiency hold great social significance.

### SUMMARY

The present disclosure provides a compound of formula (I) or a pharmaceutically acceptable salt thereof:

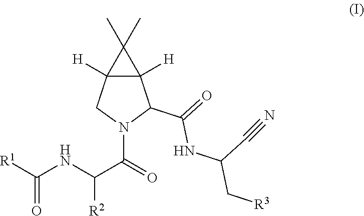

wherein
R¹ is selected from C₁-C₁₀ alkyl, wherein the C₁-C₁₀ alkyl is optionally substituted with halogen;
R² is selected from C₁-C₁₀ alkyl and (CH₂)ₙ—R⁴, wherein n is selected from 0 and 1, and R⁴ is selected from C₃-C₁₂ cycloalkyl, C₄-C₁₂ cycloalkenyl, 4- to 14-membered heterocyclyl, C₆-C₁₀ aryl, and 5- to 10-membered heteroaryl, wherein the C₁-C₁₀ alkyl, C₃-C₁₂ cycloalkyl, C₄-C₁₂ cycloalkenyl, 4- to 14-membered heterocyclyl, C₆-C₁₀ aryl, or 5- to 10-membered heteroaryl is optionally substituted with R^{2a};
R³ is selected from 4- to 14-membered heterocyclyl and 5- to 10-membered heteroaryl, wherein the 4- to 14-membered heterocyclyl or 5- to 10-membered heteroaryl is optionally substituted with R^{3a};
R^{2a} and R^{3a} are each independently selected from F, Cl, Br, I, CN, ═O, OH, NH₂, C₁-C₆ alkyl, C₃-C₆ cycloalkyl, and 4- to 7-membered heterocyclyl, wherein the OH, NH₂, C₁-C₆ alkyl, C₃-C₆ cycloalkyl, or 4- to 7-membered heterocyclyl is optionally substituted with R^{b};
each R^{b} is independently selected from F, Cl, Br, I, CN, ═O, OH, NH₂, C₁-C₆ alkyl, C₃-C₆ cycloalkyl, and 4- to 7-membered heterocyclyl, wherein the OH, NH₂, C₁-C₆ alkyl, C₃-C₆ cycloalkyl, or 4- to 7-membered heterocyclyl is optionally substituted with Re.
each R^{c} is independently selected from F, Cl, Br, I, CN, ═O, OH, NH₂, C₁-C₆ alkyl, C₃-C₆ cycloalkyl, 4- to 7-membered heterocyclyl, C₁-C₆ alkoxy, C₃-C₆ cycloalkyloxy, and 4- to 7-membered heterocyclyloxy;
the proviso is that the compound of formula (I) does not comprise

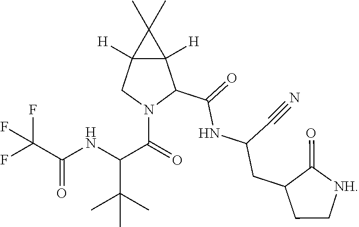 [SMILES: CC(C)(C)C(NC(=O)C(F)(F)F)C(=O)N1CC2C(C1C(=O)NC(C#N)CC1CCNC1=O)C2(C)C]

In some embodiments, R¹ is selected from C₁-C₃ alkyl, wherein the C₁-C₃ alkyl is optionally substituted with halogen.

In some embodiments, R¹ is selected from C₁-C₃ alkyl, wherein the C₁-C₃ alkyl is optionally substituted with F or Cl.

In some embodiments, R¹ is selected from CF₃ and CF₂Cl.

In some embodiments, R² is selected from C₁-C₆ alkyl and (CH₂)ₙ—R⁴, wherein n is selected from 0 and 1, and R⁴ is selected from C₅-C₁₂ cycloalkyl, 4- to 7-membered heterocyclyl, and C₆-C₁₀ aryl, wherein the C₁-C₆ alkyl, C₅-C₁₂ cycloalkyl, 4- to 7-membered heterocyclyl, or C₆-C₁₀ aryl is optionally substituted with R^{2a}.

In some embodiments, R² is selected from C₁-C₆ alkyl, C₅-C₁₂ cycloalkyl, 4- to 7-membered heterocyclyl, and CH₂—(C₆-C₁₀ aryl), wherein the C₁-C₆ alkyl, C₅-C₁₂ cycloalkyl, 4- to 7-membered heterocyclyl, or CH₂—(C₆-C₁₀ aryl) is optionally substituted with R^{2a}.

In some embodiments, R² is selected from C₁-C₄ alkyl, C₆-C₁₀ cycloalkyl, 5- to 6-membered heterocyclyl, and CH₂—(C₆-C₁₀ aryl), wherein the C₁-C₄ alkyl, C₆-C₁₀ cycloalkyl, 5- to 6-membered heterocyclyl, or CH₂—(C₆-C₁₀ aryl) is optionally substituted with R^{2a}.

In some embodiments, R² is selected from C₁-C₄ alkyl, C₆-C₁₀ cycloalkyl, and CH₂—(C₆-C₁₀ aryl), wherein the C₁-C₄ alkyl, C₆-C₁₀ cycloalkyl, or CH₂—(C₆-C₁₀ aryl) is optionally substituted with R^{2a}.

In some embodiments, R^{2a} is selected from F, Cl, Br, I, CN, ═O, OH, NH₂, and C₁-C₆ alkyl, wherein the OH, NH₂, or C₁-C₆ alkyl is optionally substituted with R^{b}.

In some embodiments, R^{2a} is selected from OH, C₁-C₃ alkyl, and NH₂, wherein the NH₂ is optionally substituted with R^{b}.

In some embodiments, R^{b} is selected from C₁-C₃ alkyl.

In some embodiments, R^{2a} is selected from OH, methyl, and N(CH₃)₂.

In some embodiments, R^{2a} is selected from OH and N(CH₃)₂.

In some embodiments R² is selected from

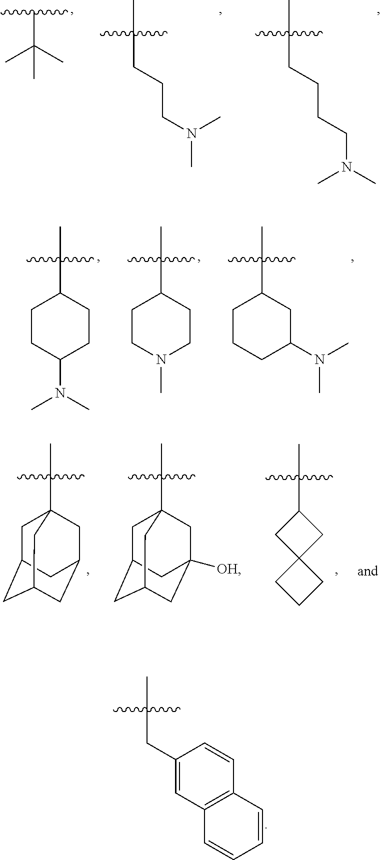

In some embodiments R² is selected from

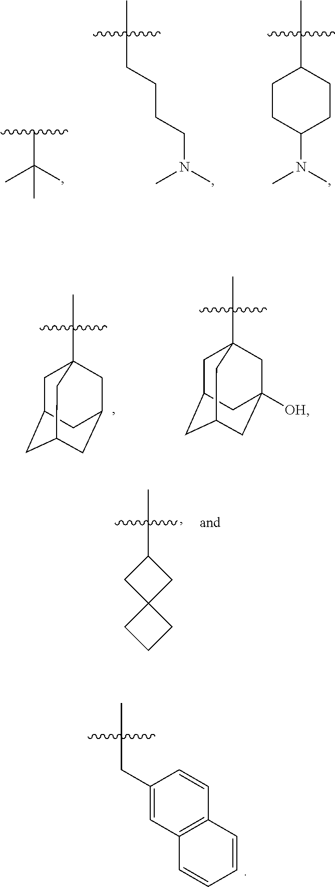

In some embodiments, R³ is selected from 4- to 14-membered heterocyclyl, wherein the 4- to 14-membered heterocyclyl is substituted with R^{3a}.

In some embodiments, R³ is selected from 4- to 7-membered monocyclic heterocyclyl, 6- to 14-membered fused heterocyclyl, 6- to 14-membered spiro heterocyclyl, and 6- to 14-membered bridged heterocyclyl, wherein the 4- to 7-membered monocyclic heterocyclyl, 6- to 14-membered fused heterocyclyl, 6- to 14-membered spiro heterocyclyl, or 6- to 14-membered bridged heterocyclyl is optionally substituted with R^{3a}.

In some embodiments, R³ is selected from 4- to 7-membered monocyclic heterocyclyl and 6- to 14-membered fused heterocyclyl, wherein the 4- to 7-membered monocyclic heterocyclyl or 6- to 14-membered fused heterocyclyl is optionally substituted with R^{3a}.

In some embodiments, R³ is selected from

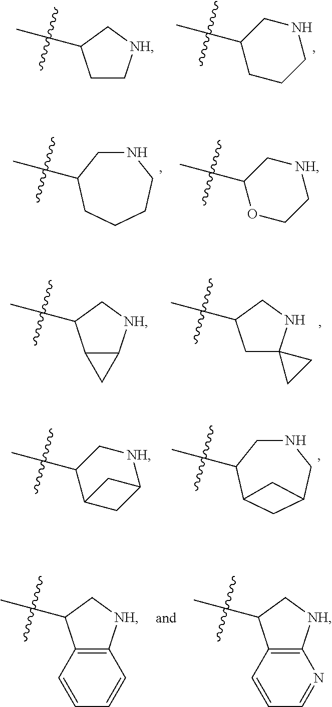

wherein the

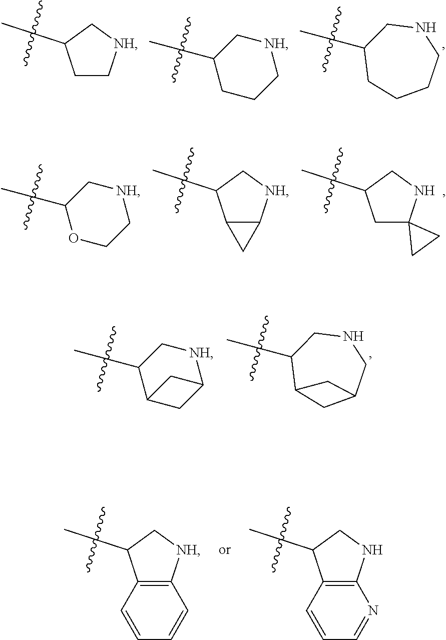

is optionally substituted with R^{3a}.

In some embodiments, R³ is selected from

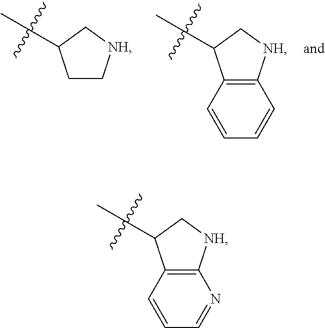 [SMILES: *C1CCNC1 ; *C1CNc2ccccc21 ; *C1CNc2ncccc21]

wherein the

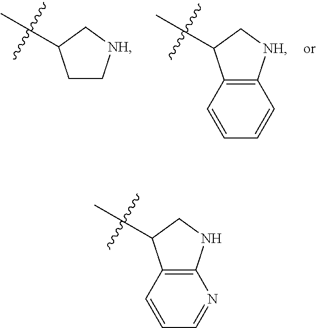 [SMILES: *C1CCNC1 ; *C1CNc2ccccc21 ; *C1CNc2ncccc21]

or is optionally substituted with R^{3a}.

In some embodiments, R^{3a} is independently selected from F, Cl, Br, I, CN, ═O, OH, NH₂, C₁-C₆ alkyl, C₃-C₆ cycloalkyl, and 4- to 7-membered heterocyclyl.

In some embodiments, R^{3a} is independently selected from F, Cl, Br, I, CN, ═O, and C₁-C₆ alkyl.

In some embodiments, R^{3a} is independently selected from F, Cl, and ═O.

In some embodiments, R^{3a} is selected from ═O.

In some embodiments, R³ is selected from

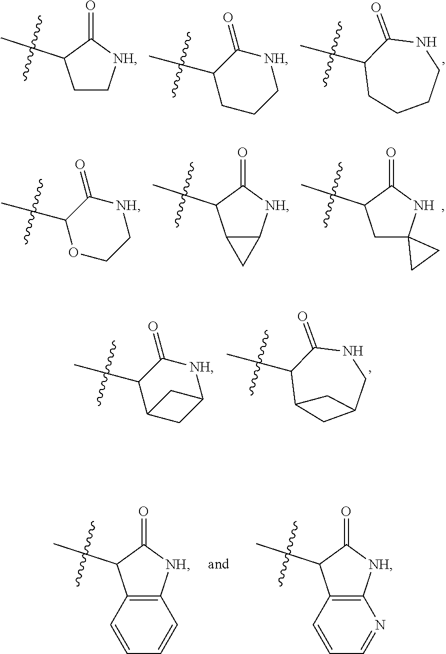

wherein the

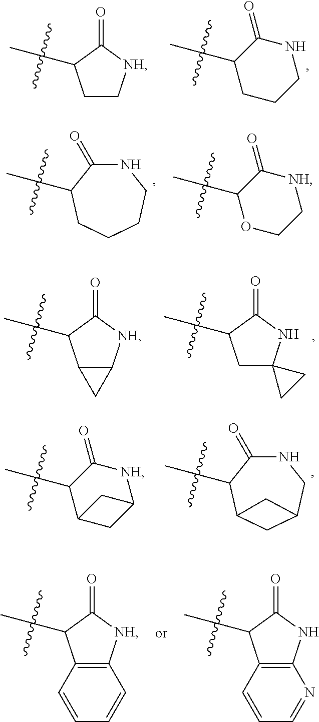

is optionally substituted with F, Cl, Br, I, CN, or C₁-C₃ alkyl.

In some embodiments, R³ is selected from

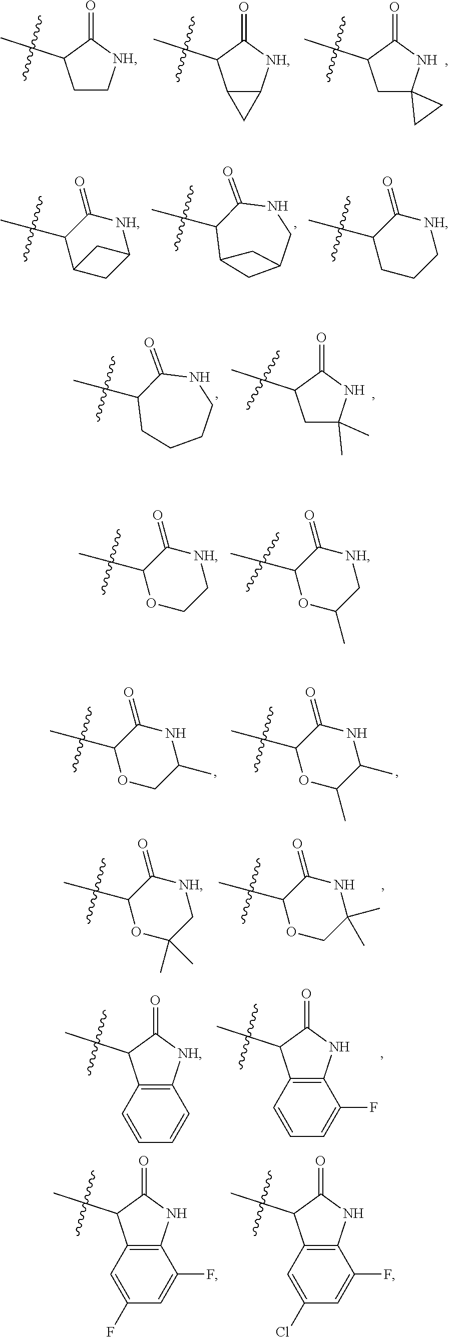

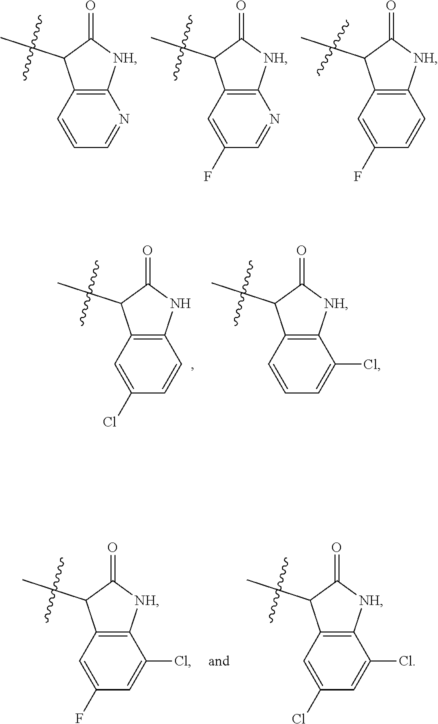

In some embodiments, R³ is selected from

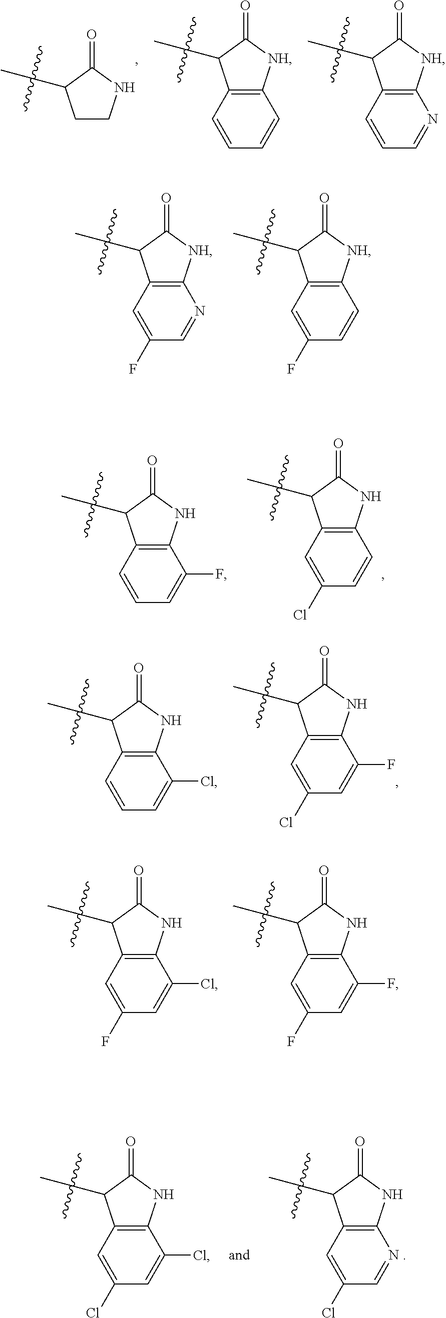

In some embodiments, R³ is selected from

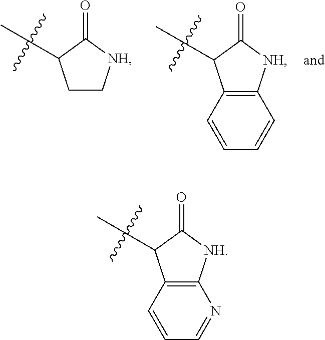 [SMILES: *C1CCNC1=O ; *C1C(=O)Nc2ccccc21 ; *C1C(=O)Nc2ncccc21]

In some embodiments, the compound of formula (I) or the pharmaceutically acceptable salt thereof is selected from a compound of formula (Ia) or a pharmaceutically acceptable salt thereof:

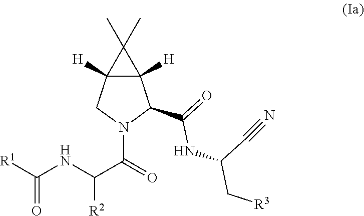

wherein R¹, R², and R³ are as defined above.

In some embodiments, the compound of formula (I) or the pharmaceutically acceptable salt thereof is selected from a compound of formula (Ib) or a pharmaceutically acceptable salt thereof:

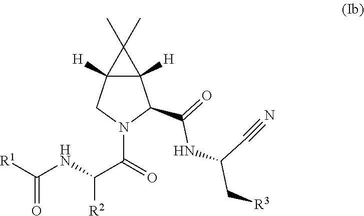

wherein R¹, R², and R³ are as defined above.

In some embodiments, the compound of formula (I) or the pharmaceutically acceptable salt thereof is selected from a compound of formula (Ic) or a pharmaceutically acceptable salt thereof:

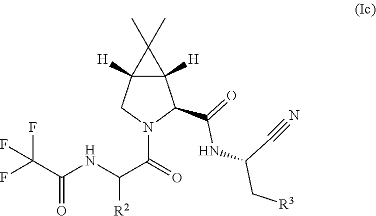

wherein R² and R³ are as defined above.

In some embodiments, the compound of formula (I) or the pharmaceutically acceptable salt thereof is selected from a compound of formula (Id) or a pharmaceutically acceptable salt thereof:

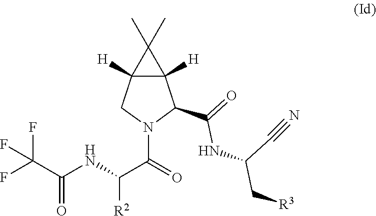

wherein R² and R³ are as defined above.

In some embodiments, the compound of formula (I) or the pharmaceutically acceptable salt thereof is selected from the following compounds or pharmaceutically acceptable salts thereof:

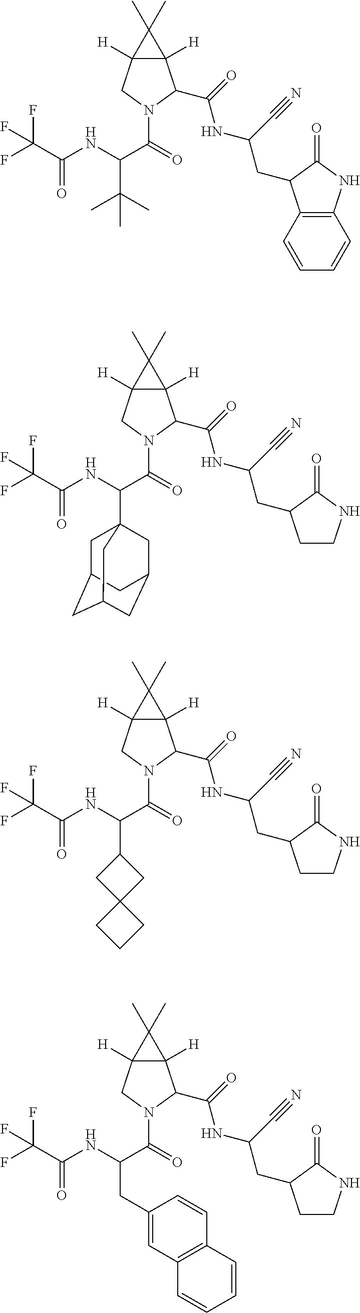

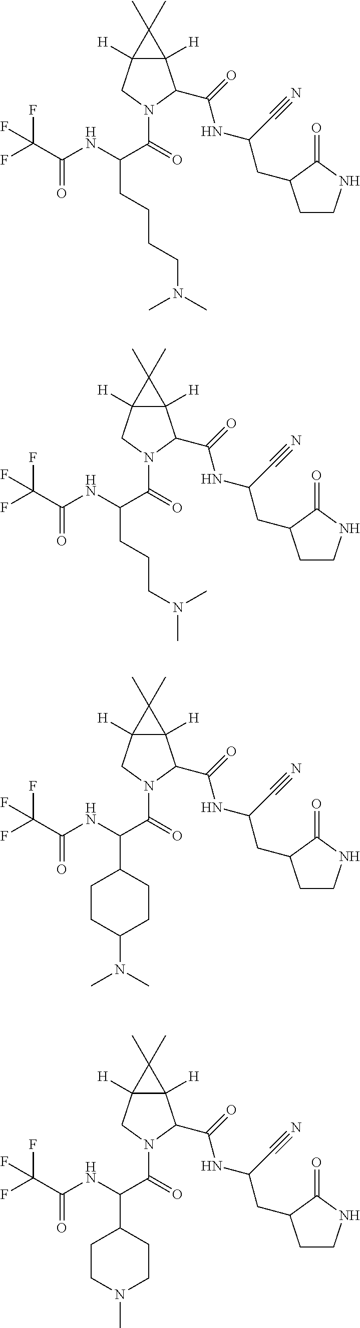

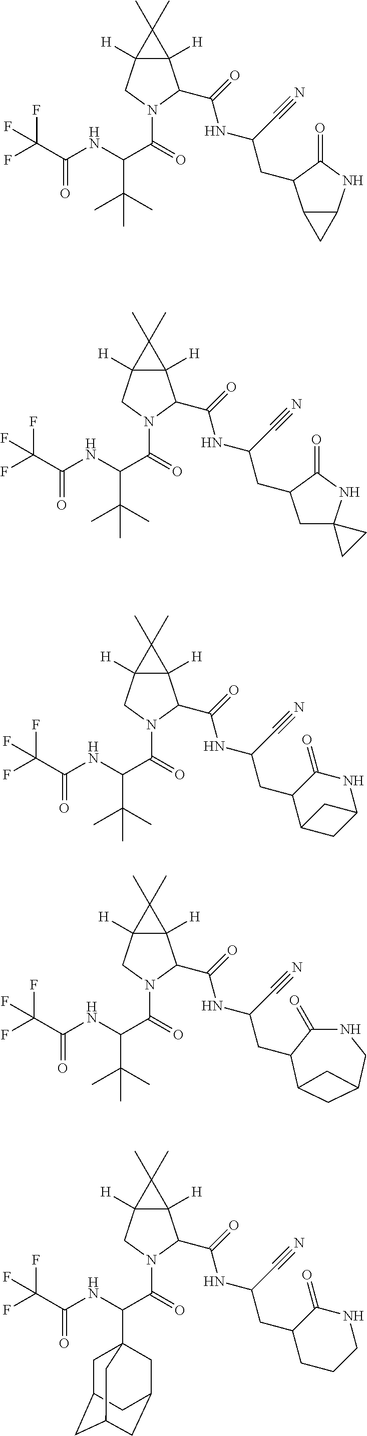

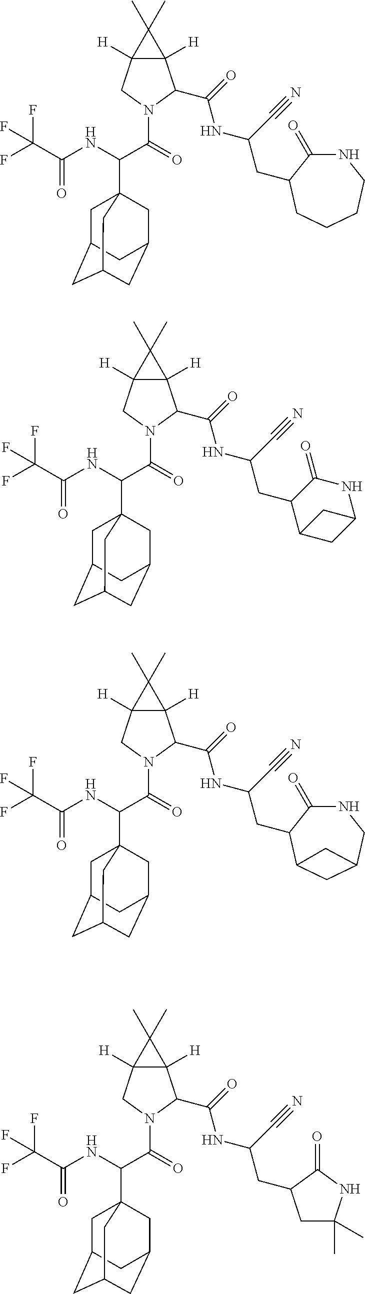

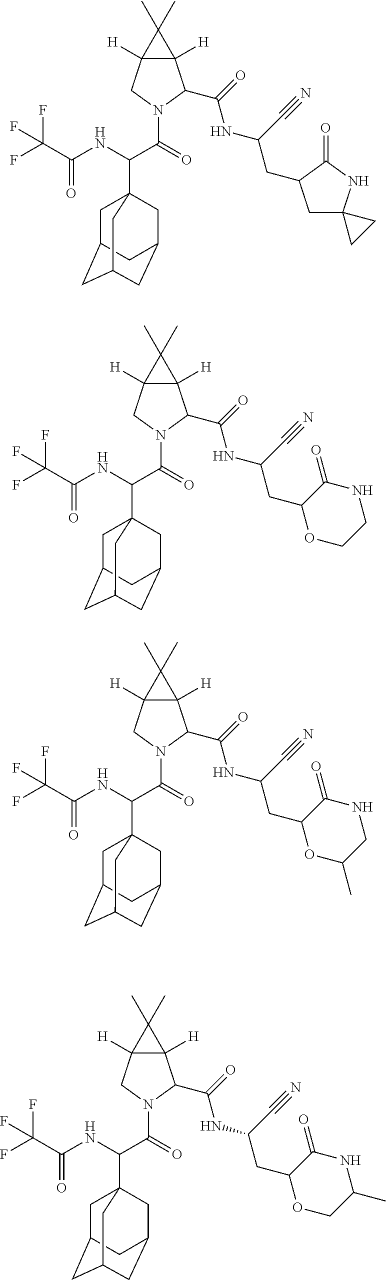

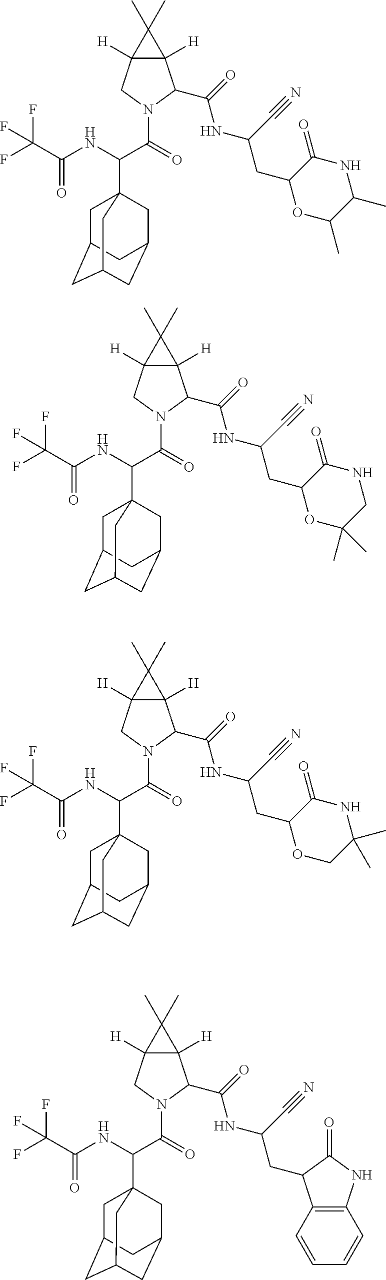

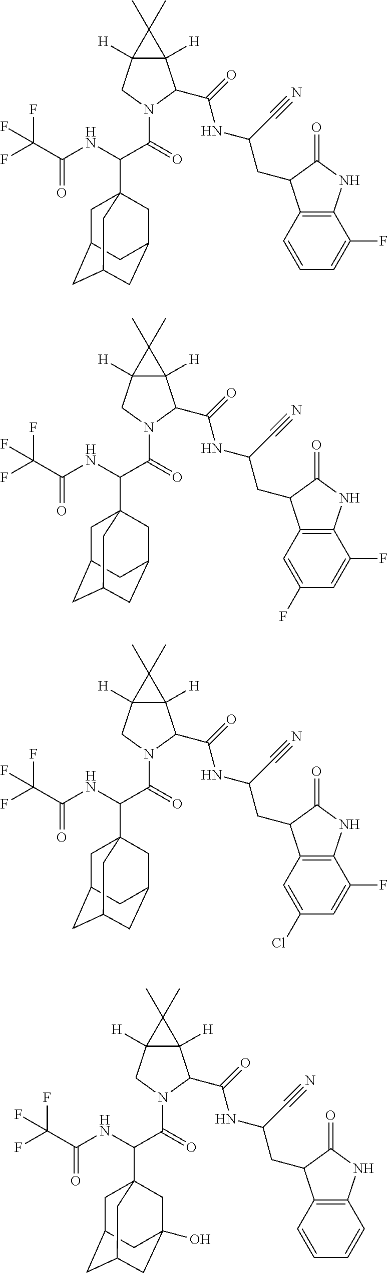

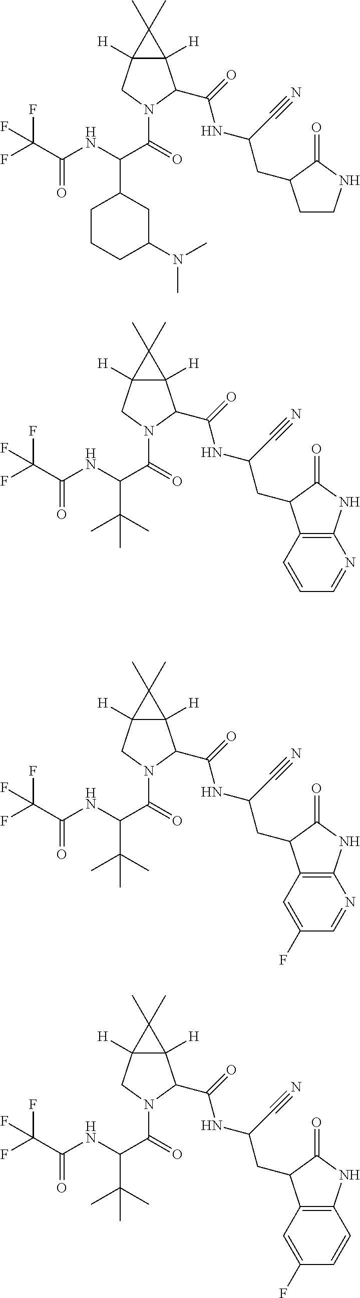

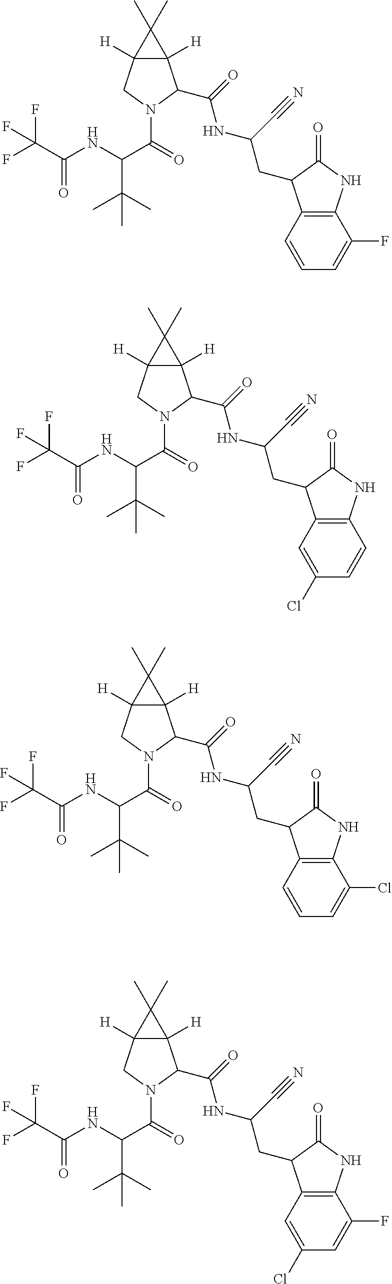 [SMILES: CC(C)(C)C(NC(=O)C(F)(F)F)C(=O)N1CC2C(C1C(=O)NC(C#N)CC1C(=O)Nc3c(F)cccc31)C2(C)C ; CC(C)(C)C(NC(=O)C(F)(F)F)C(=O)N1CC2C(C1C(=O)NC(C#N)CC1C(=O)Nc3ccc(Cl)cc31)C2(C)C ; CC(C)(C)C(NC(=O)C(F)(F)F)C(=O)N1CC2C(C1C(=O)NC(C#N)CC1C(=O)Nc3c(Cl)cccc31)C2(C)C ; CC(C)(C)C(NC(=O)C(F)(F)F)C(=O)N1CC2C(C1C(=O)NC(C#N)CC1C(=O)Nc3c(F)cc(Cl)cc31)C2(C)C]

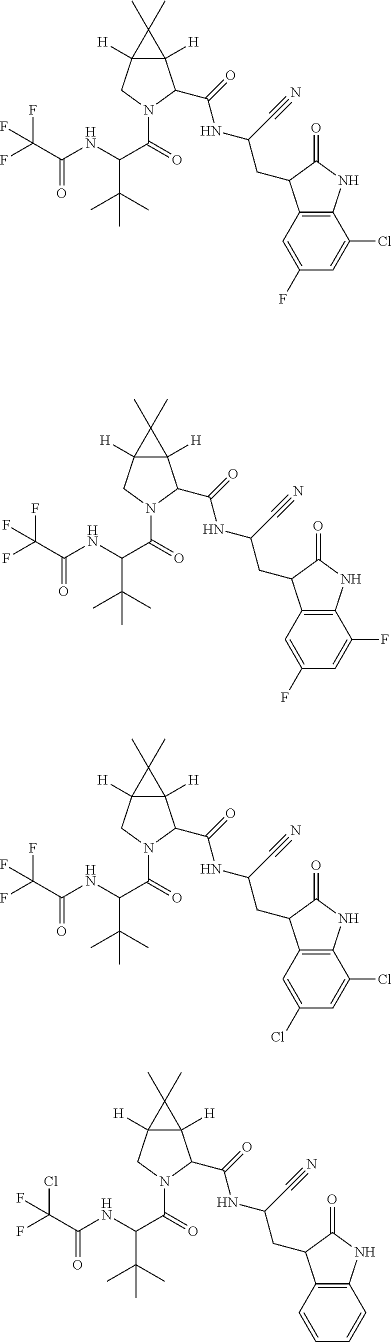 [SMILES: CC(C)(C)C(NC(=O)C(F)(F)F)C(=O)N1CC2C(C1C(=O)NC(C#N)CC1C(=O)Nc3c(Cl)cc(F)cc31)C2(C)C ; CC(C)(C)C(NC(=O)C(F)(F)F)C(=O)N1CC2C(C1C(=O)NC(C#N)CC1C(=O)Nc3c(F)cc(F)cc31)C2(C)C ; CC(C)(C)C(NC(=O)C(F)(F)F)C(=O)N1CC2C(C1C(=O)NC(C#N)CC1C(=O)Nc3c(Cl)cc(Cl)cc31)C2(C)C ; CC(C)(C)C(NC(=O)C(F)(F)Cl)C(=O)N1CC2C(C1C(=O)NC(C#N)CC1C(=O)Nc3ccccc31)C2(C)C]

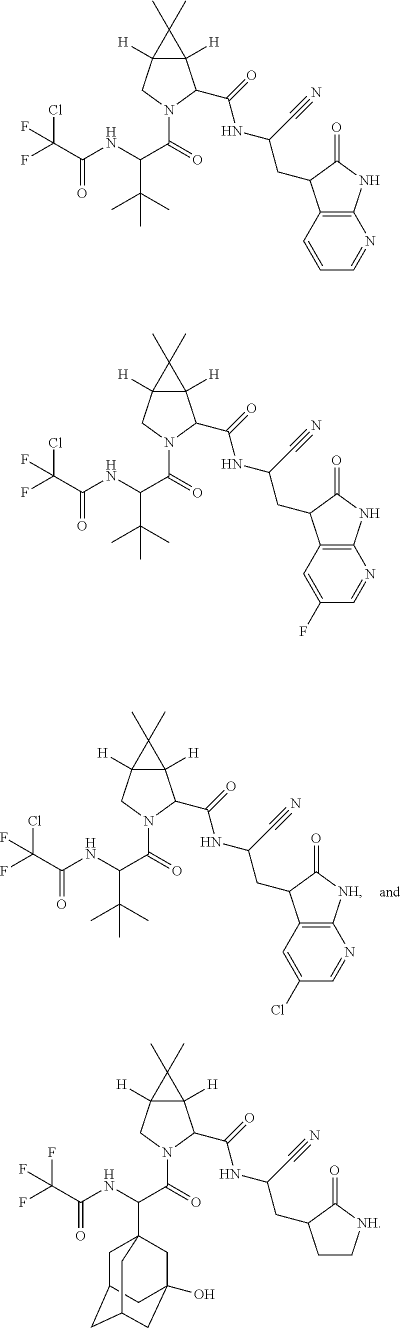 [SMILES: CC(C)(C)C(NC(=O)C(F)(F)Cl)C(=O)N1CC2C(C1C(=O)NC(C#N)CC1C(=O)Nc3ncccc31)C2(C)C ; CC(C)(C)C(NC(=O)C(F)(F)Cl)C(=O)N1CC2C(C1C(=O)NC(C#N)CC1C(=O)Nc3ncc(F)cc31)C2(C)C ; CC(C)(C)C(NC(=O)C(F)(F)Cl)C(=O)N1CC2C(C1C(=O)NC(C#N)CC1C(=O)Nc3ncc(Cl)cc31)C2(C)C ; CC1(C)C2CN(C(=O)C(NC(=O)C(F)(F)F)C34CC5CC(CC(O)(C5)C3)C4)C(C(=O)NC(C#N)CC3CCNC3=O)C21]

In some embodiments, the compound of formula (I) or the pharmaceutically acceptable salt thereof is selected from the following compounds or pharmaceutically acceptable salts thereof:

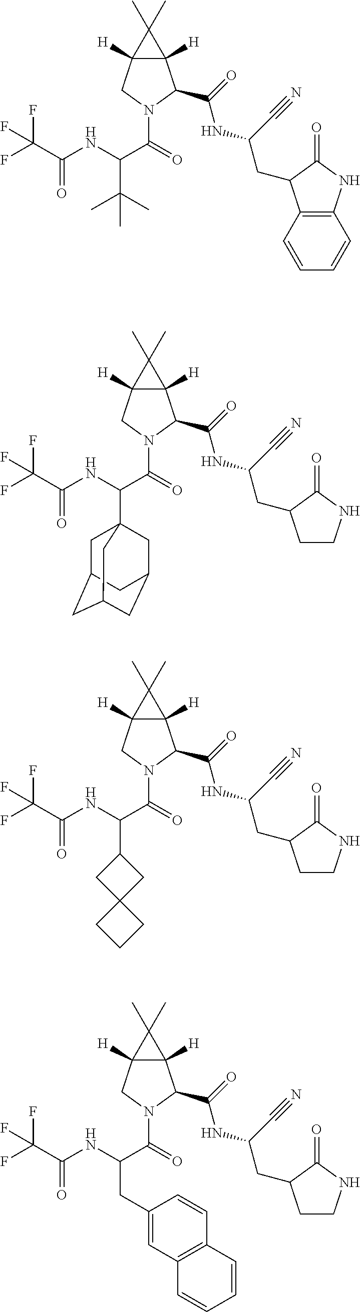

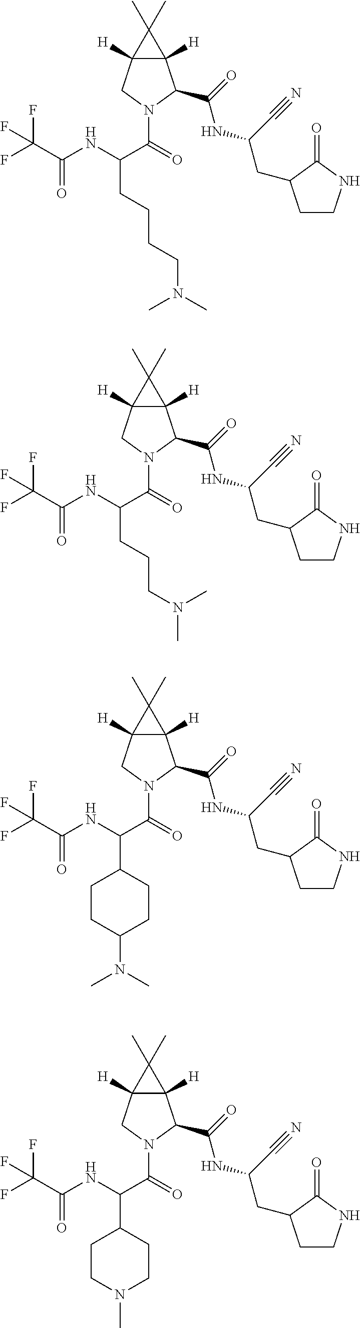

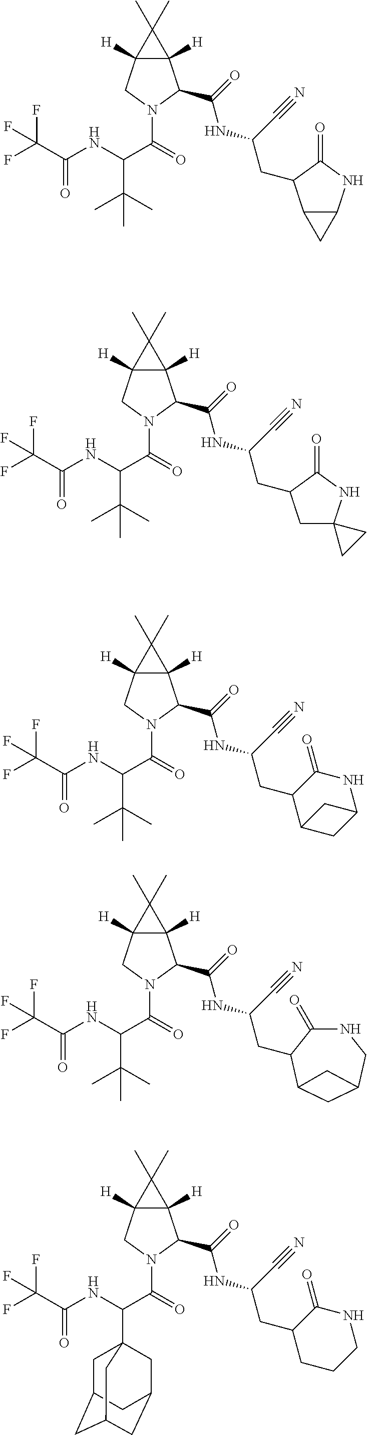

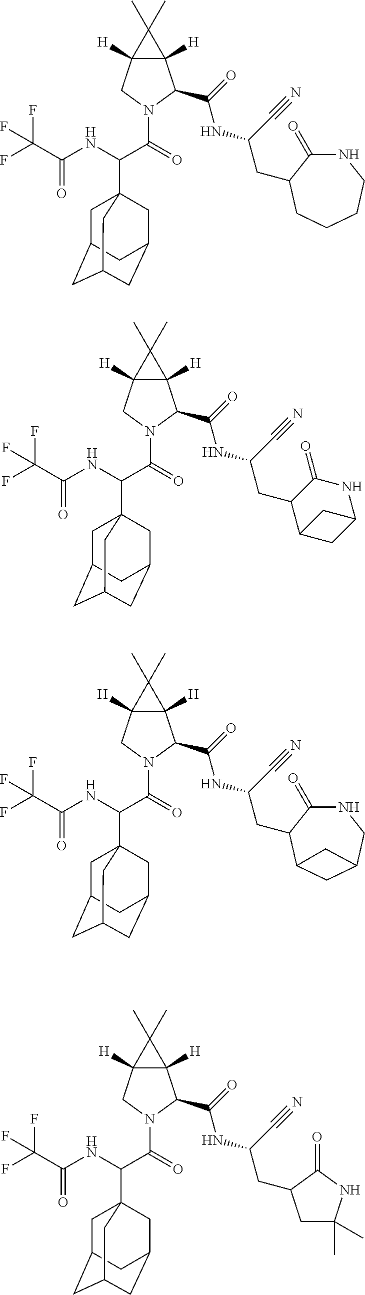

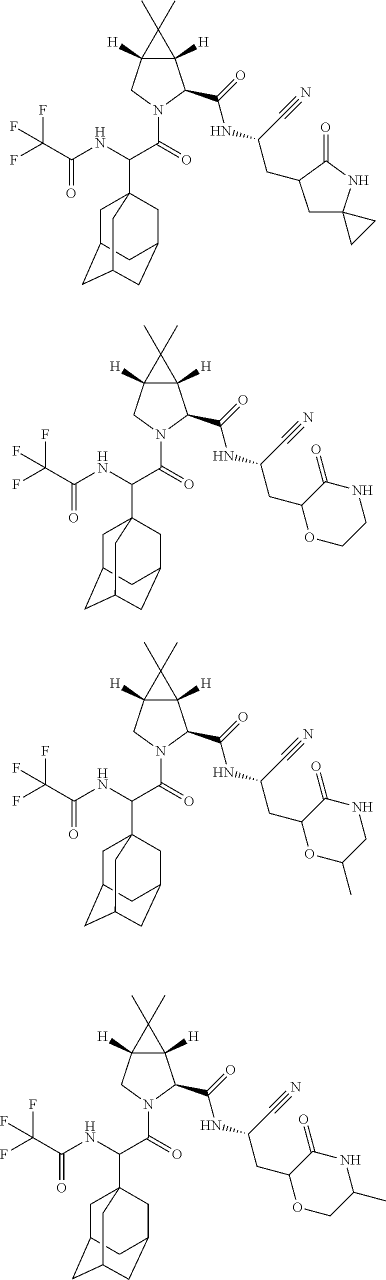

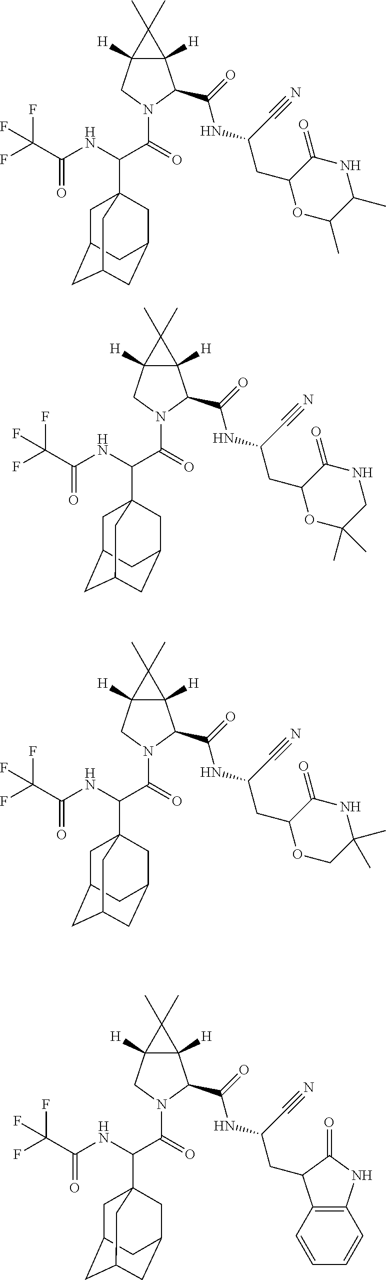

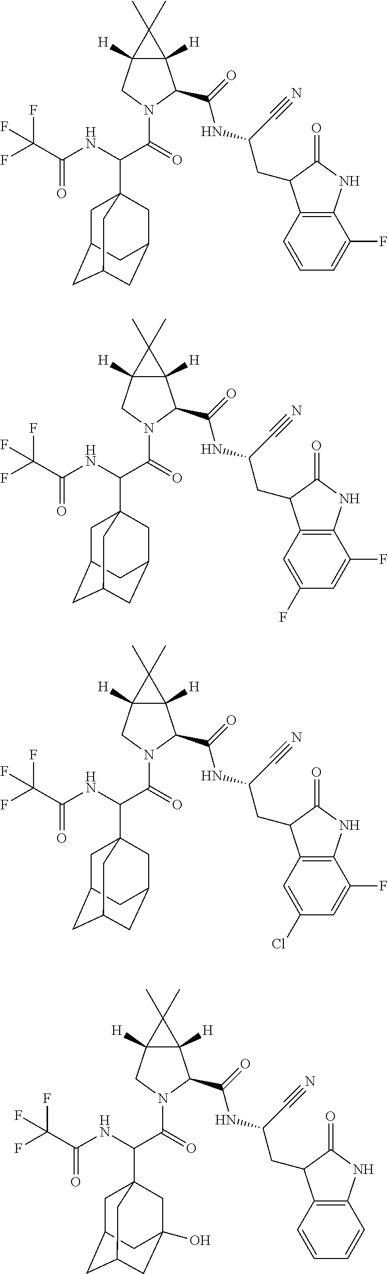

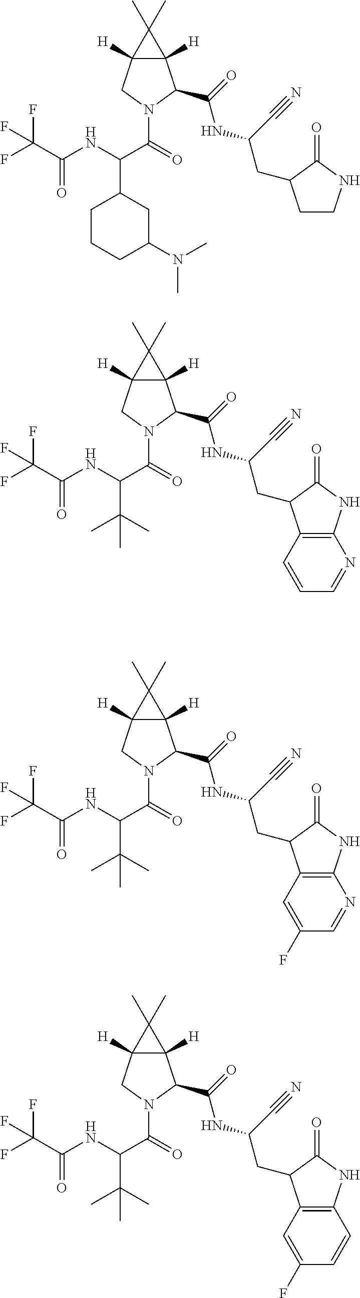

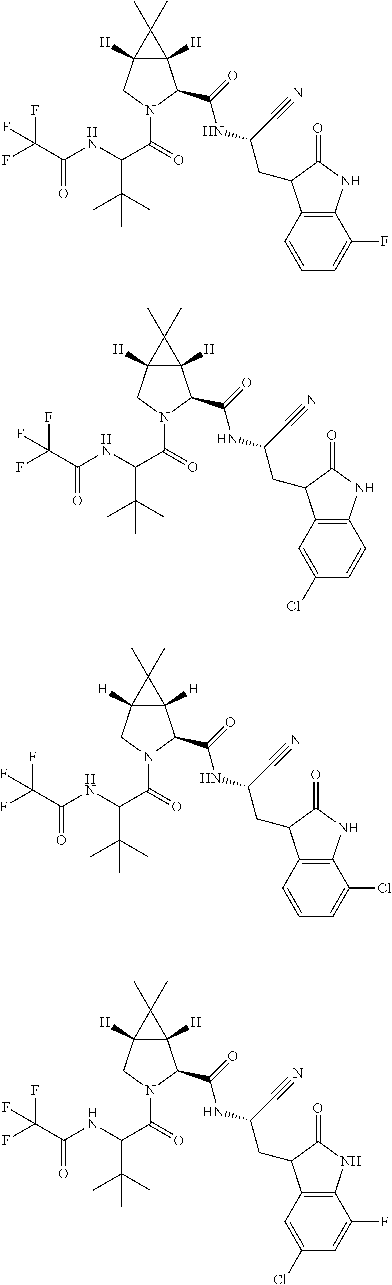 [SMILES: CC(C)(C)C(NC(=O)C(F)(F)F)C(=O)N1C[C@H]2[C@@H]([C@H]1C(=O)N[C@H](C#N)CC1C(=O)Nc3c(F)cccc31)C2(C)C ; CC(C)(C)C(NC(=O)C(F)(F)F)C(=O)N1C[C@H]2[C@@H]([C@H]1C(=O)N[C@H](C#N)CC1C(=O)Nc3ccc(Cl)cc31)C2(C)C ; CC(C)(C)C(NC(=O)C(F)(F)F)C(=O)N1C[C@H]2[C@@H]([C@H]1C(=O)N[C@H](C#N)CC1C(=O)Nc3c(Cl)cccc31)C2(C)C ; CC(C)(C)C(NC(=O)C(F)(F)F)C(=O)N1C[C@H]2[C@@H]([C@H]1C(=O)N[C@H](C#N)CC1C(=O)Nc3c(F)cc(Cl)cc31)C2(C)C]

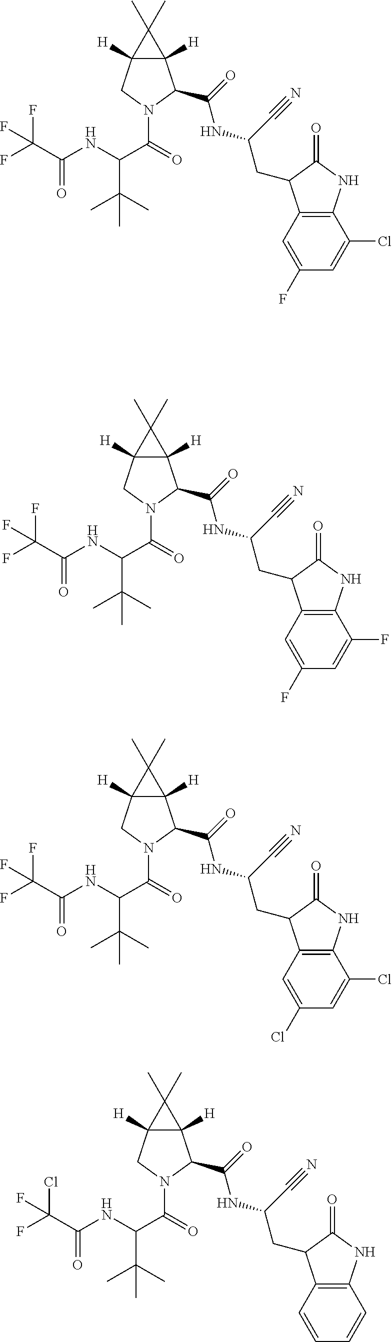 [SMILES: CC(C)(C)C(NC(=O)C(F)(F)F)C(=O)N1C[C@H]2[C@@H]([C@H]1C(=O)N[C@H](C#N)CC1C(=O)Nc3c(Cl)cc(F)cc31)C2(C)C ; CC(C)(C)C(NC(=O)C(F)(F)F)C(=O)N1C[C@H]2[C@@H]([C@H]1C(=O)N[C@H](C#N)CC1C(=O)Nc3c(F)cc(F)cc31)C2(C)C ; CC(C)(C)C(NC(=O)C(F)(F)F)C(=O)N1C[C@H]2[C@@H]([C@H]1C(=O)N[C@H](C#N)CC1C(=O)Nc3c(Cl)cc(Cl)cc31)C2(C)C ; CC(C)(C)C(NC(=O)C(F)(F)Cl)C(=O)N1C[C@H]2[C@@H]([C@H]1C(=O)N[C@H](C#N)CC1C(=O)Nc3ccccc31)C2(C)C]

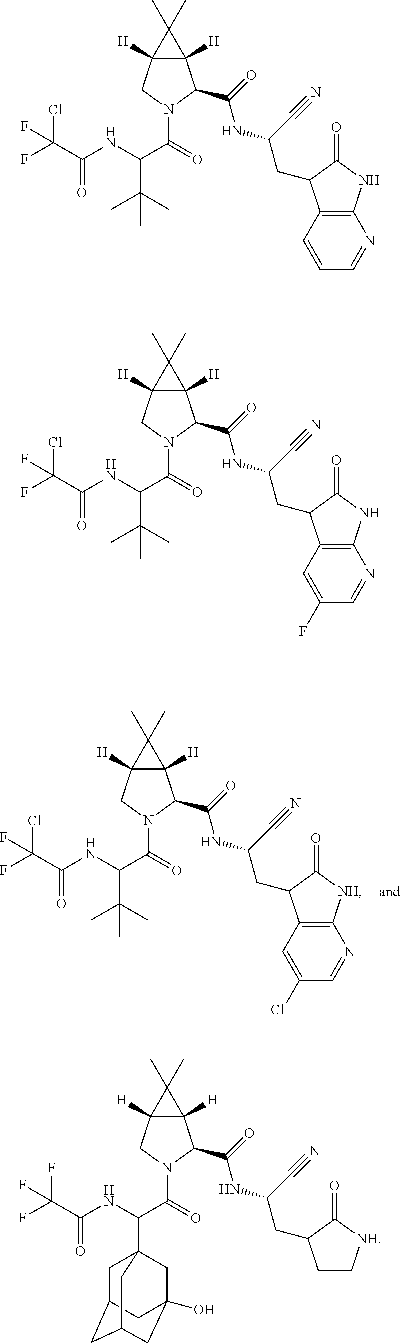 [SMILES: CC(C)(C)C(NC(=O)C(F)(F)Cl)C(=O)N1C[C@H]2[C@@H]([C@H]1C(=O)N[C@H](C#N)CC1C(=O)Nc3ncccc31)C2(C)C ; CC(C)(C)C(NC(=O)C(F)(F)Cl)C(=O)N1C[C@H]2[C@@H]([C@H]1C(=O)N[C@H](C#N)CC1C(=O)Nc3ncc(F)cc31)C2(C)C ; CC(C)(C)C(NC(=O)C(F)(F)Cl)C(=O)N1C[C@H]2[C@@H]([C@H]1C(=O)N[C@H](C#N)CC1C(=O)Nc3ncc(Cl)cc31)C2(C)C ; CC1(C)[C@@H]2[C@@H](C(=O)N[C@H](C#N)CC3CCNC3=O)N(C(=O)C(NC(=O)C(F)(F)F)C34CC5CC(CC(O)(C5)C3)C4)C[C@@H]21]

In some embodiments, the compound of formula (I) or the pharmaceutically acceptable salt thereof is selected from the following compounds or pharmaceutically acceptable salts thereof.

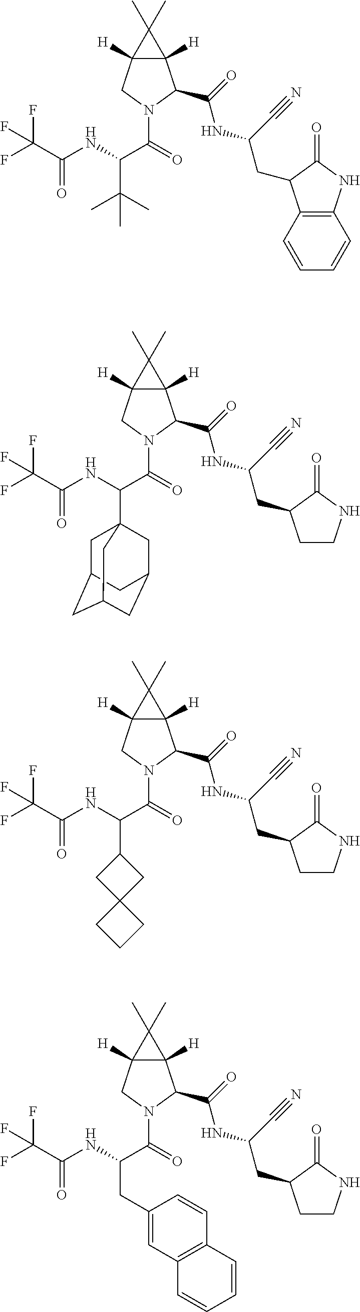

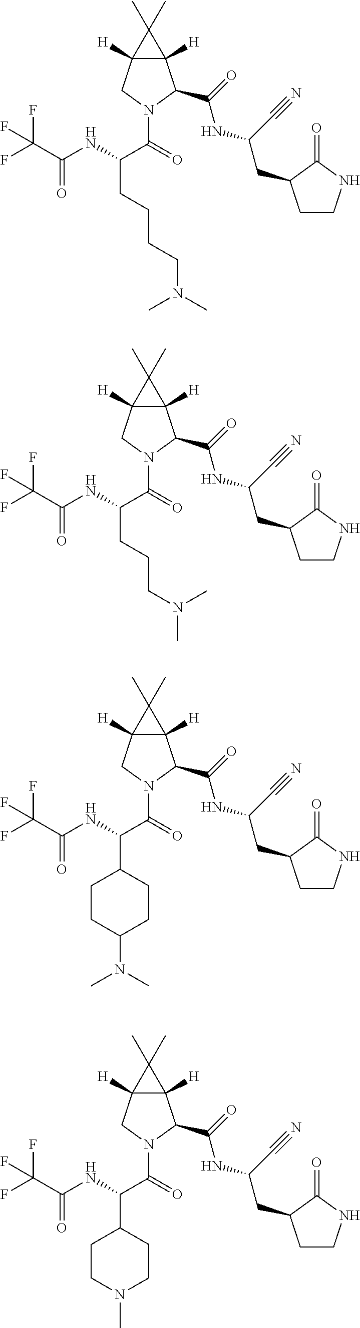

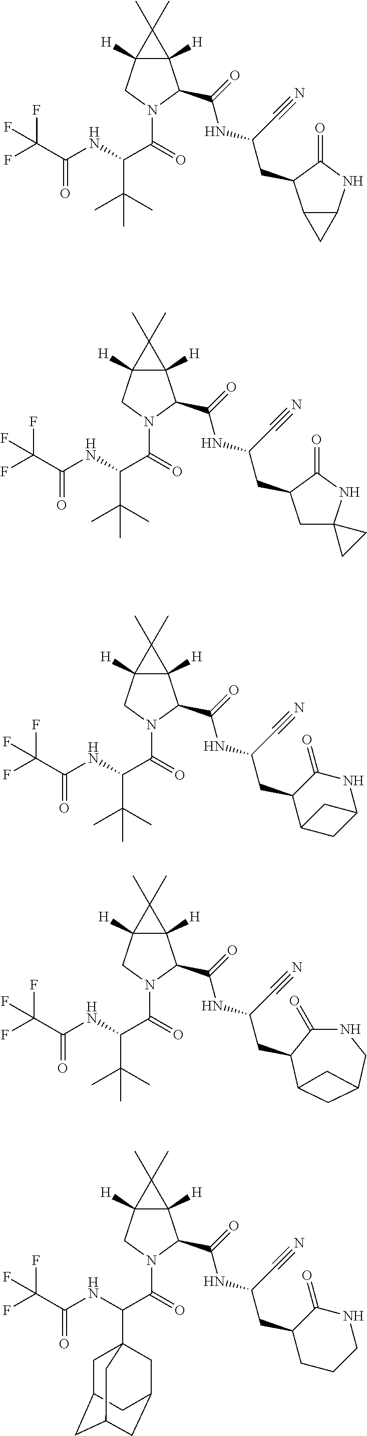

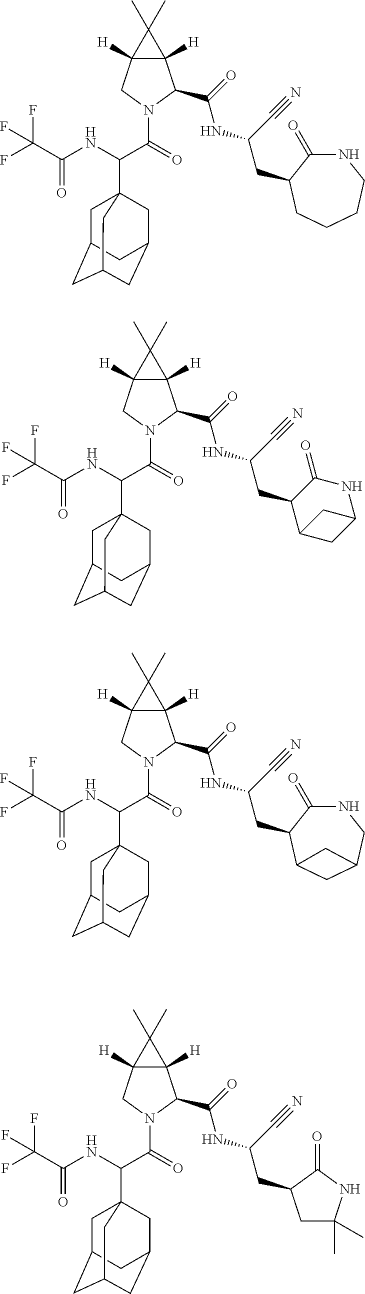

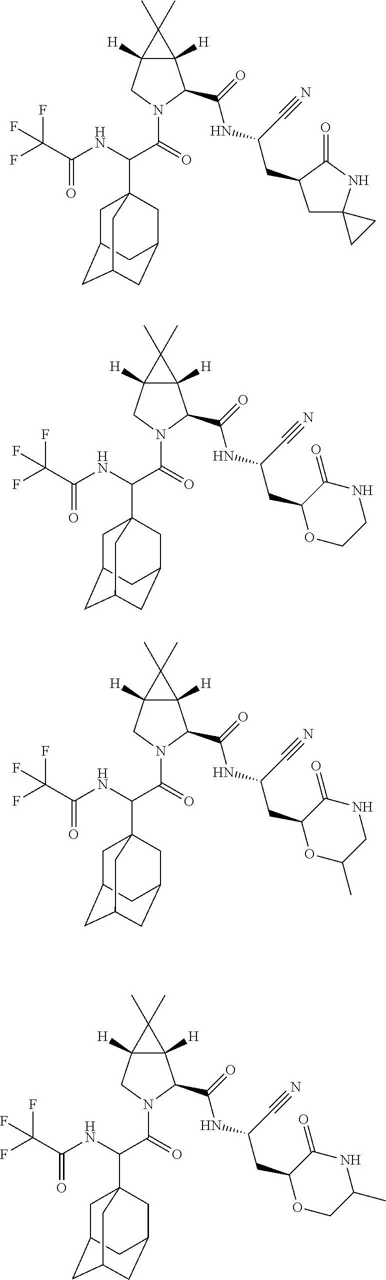

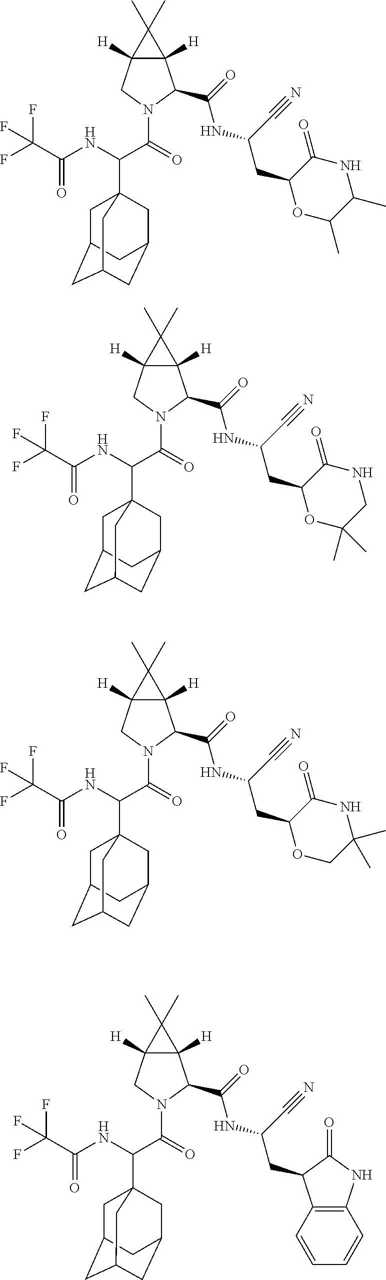

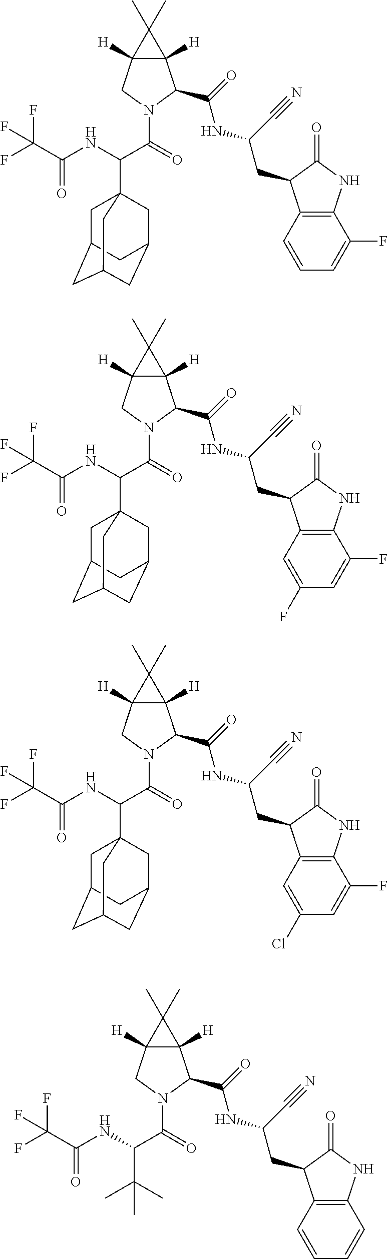

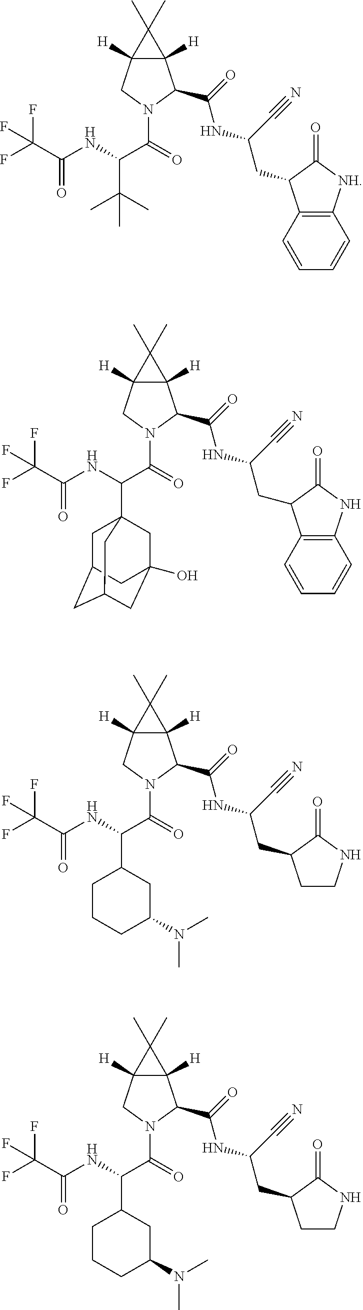 [SMILES: CC(C)(C)[C@H](NC(=O)C(F)(F)F)C(=O)N1C[C@H]2[C@@H]([C@H]1C(=O)N[C@H](C#N)C[C@@H]1C(=O)Nc3ccccc31)C2(C)C ; CC1(C)[C@@H]2[C@@H](C(=O)N[C@H](C#N)CC3C(=O)Nc4ccccc43)N(C(=O)C(NC(=O)C(F)(F)F)C34CC5CC(CC(O)(C5)C3)C4)C[C@@H]21 ; CN(C)[C@@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1 ; CN(C)[C@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1]

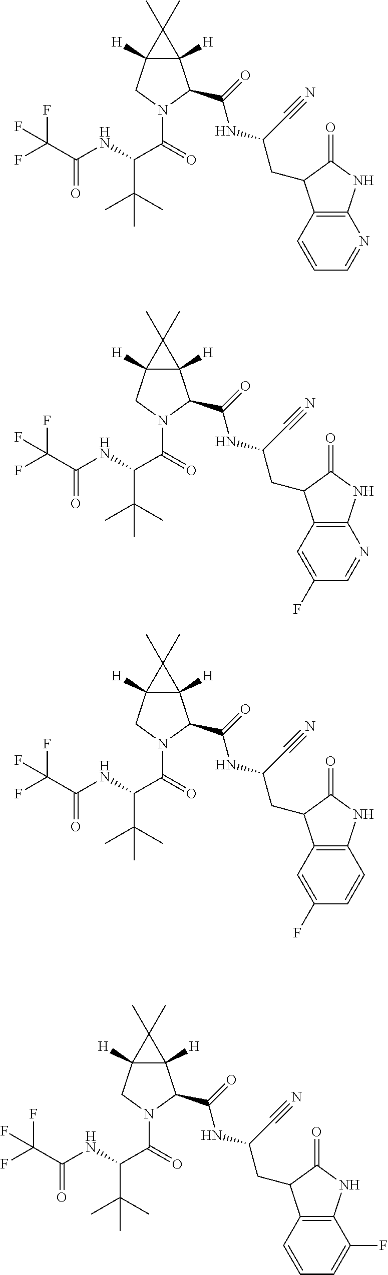 [SMILES: CC(C)(C)[C@H](NC(=O)C(F)(F)F)C(=O)N1C[C@H]2[C@@H]([C@H]1C(=O)N[C@H](C#N)CC1C(=O)Nc3ncccc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3ncc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3ccc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(F)cccc31)C2(C)C]

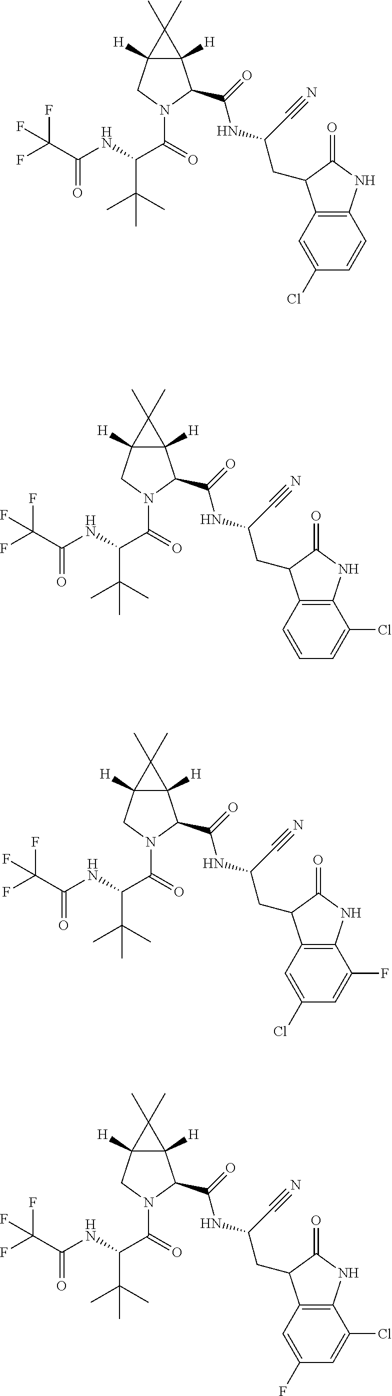 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(Cl)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cccc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(F)cc(Cl)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(F)cc31)C2(C)C]

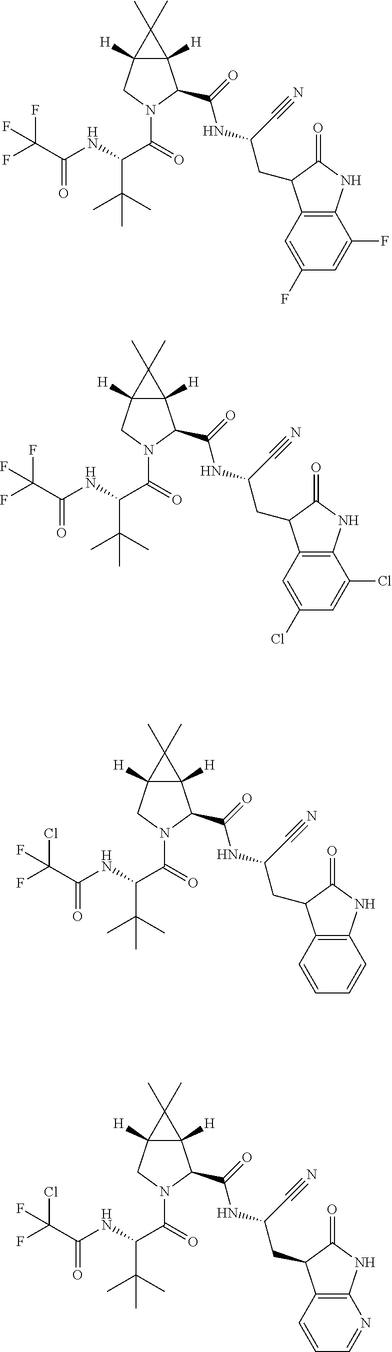 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(F)cc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(Cl)cc31)C2(C)C ; CC(C)(C)[C@H](NC(=O)C(F)(F)Cl)C(=O)N1C[C@H]2[C@@H]([C@H]1C(=O)N[C@H](C#N)CC1C(=O)Nc3ccccc31)C2(C)C ; CC(C)(C)[C@H](NC(=O)C(F)(F)Cl)C(=O)N1C[C@H]2[C@@H]([C@H]1C(=O)N[C@H](C#N)C[C@H]1C(=O)Nc3ncccc31)C2(C)C]

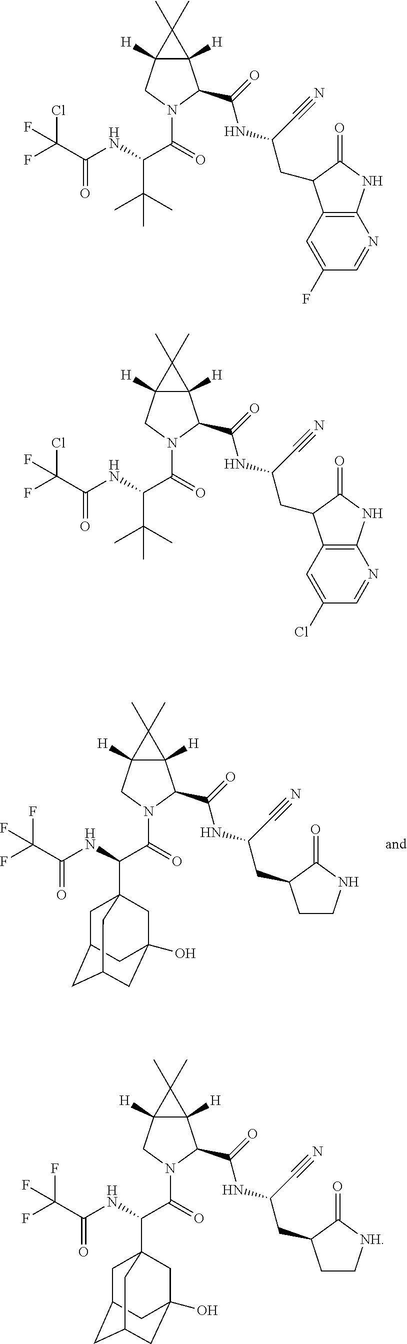 [SMILES: CC(C)(C)[C@H](NC(=O)C(F)(F)Cl)C(=O)N1C[C@H]2[C@@H]([C@H]1C(=O)N[C@H](C#N)CC1C(=O)Nc3ncc(F)cc31)C2(C)C ; CC(C)(C)[C@H](NC(=O)C(F)(F)Cl)C(=O)N1C[C@H]2[C@@H]([C@H]1C(=O)N[C@H](C#N)CC1C(=O)Nc3ncc(Cl)cc31)C2(C)C ; CC1(C)[C@@H]2[C@@H](C(=O)N[C@H](C#N)C[C@@H]3CCNC3=O)N(C(=O)[C@H](NC(=O)C(F)(F)F)C34CC5CC(CC(O)(C5)C3)C4)C[C@@H]21 ; CC1(C)[C@@H]2[C@@H](C(=O)N[C@H](C#N)C[C@@H]3CCNC3=O)N(C(=O)[C@@H](NC(=O)C(F)(F)F)C34CC5CC(CC(O)(C5)C3)C4)C[C@@H]21]

The present disclosure also provides a pharmaceutical composition comprising the compound of formula (I) or the pharmaceutically acceptable salt thereof and a pharmaceutically acceptable excipient.

Further, the present disclosure relates to use of the compound of formula (I) or the pharmaceutically acceptable salt thereof, or the pharmaceutical composition thereof in the preparation of a medicament for preventing or treating a coronavirus 3CL protease-related disease.

Further, the present disclosure relates to use of the compound of formula (I) or the pharmaceutically acceptable salt thereof, or the pharmaceutical composition thereof in the prevention or treatment of a coronavirus 3CL protease-related disease.

Further, the present disclosure relates to the compound of formula (I) or the pharmaceutically acceptable salt thereof, or the pharmaceutical composition thereof for use in the prevention or treatment of a coronavirus 3CL protease-related disease.

The present disclosure also relates to a method for preventing or treating a coronavirus 3CL protease-related disease, comprising administering to a patient a therapeutically effective amount of a pharmaceutical formulation comprising the compound of formula (I) or the pharmaceutically acceptable salt thereof described herein.

Further, the present disclosure relates to use of the compound of formula (I) or the pharmaceutically acceptable salt thereof, or the pharmaceutical composition thereof in the preparation of a medicament for preventing or treating a related disease caused by coronavirus infection.

Further, the present disclosure relates to use of the compound of formula (I) or the pharmaceutically acceptable salt thereof, or the pharmaceutical composition thereof in the prevention or treatment of a related disease caused by coronavirus infection.

Further, the present disclosure relates to the compound of formula (I) or the pharmaceutically acceptable salt thereof, or the pharmaceutical composition thereof for use in the prevention or treatment of a related disease caused by coronavirus infection.

The present disclosure also relates to a method for preventing or treating a related disease caused by coronavirus infection, comprising administering to a patient a therapeutically effective amount of a pharmaceutical formulation comprising the compound of formula (I) or the pharmaceutically acceptable salt thereof described herein.

In some embodiments, the coronavirus is selected from severe acute respiratory syndrome coronavirus (SARS-CoV), Middle East respiratory syndrome coronavirus (MERS-CoV), and 2019 novel coronavirus (2019-nCoV or SARS-CoV-2).

In some embodiments, the coronavirus 3CL protease-related disease is selected from respiratory tract infection, pneumonia, and complications thereof.

In some embodiments, the related disease caused by coronavirus infection is selected from respiratory tract infection, pneumonia, and complications thereof.

### Definitions and Description

Unless otherwise stated, the terms used in the present disclosure have the following meanings, and the definitions of groups and terms described in the present disclosure, including definitions thereof as examples, exemplary definitions, preferred definitions, definitions documented in tables, definitions of specific compounds in the examples, and the like, may be arbitrarily combined and incorporated with each other. A certain term, unless otherwise specifically defined, should not be considered indefinite or unclear, but should be understood according to its common meaning in the field. When referring to a trade name, it is intended to refer to its corresponding commercial product or its active ingredient.

Herein, “ [symbol] ” represents a connection site.

The illustration method for the racemic or enantiomerically pure compounds herein is from Maehr, J Chem. Ed. 1985, 62:114-120. Unless otherwise stated, the absolute configuration of a stereogenic center is represented by a wedged bond and a wedged dashed bond ( [symbol] and [symbol] ), and the relative configuration of a stereogenic center (e.g., cis- or trans-configuration of alicyclic compounds) is represented by a black solid bond and a dashed bond ( [symbol] and [symbol] ).

The term “tautomer” refers to functional isomers resulting from the rapid movement of an atom in a molecule between two positions. The compounds of the present disclosure may exhibit the tautomerism. Tautomeric compounds may exist in two or more interconvertible forms. Tautomers generally exist in an equilibrium form. Trying to separate a single tautomer usually leads to a mixture, the physicochemical properties of which are consistent with the mixture of the compound. The position of the equilibrium depends on the chemical properties of the molecule. For example, in many aliphatic aldehydes and ketones such as acetaldehyde, the keto form predominates; whereas in phenol, the enol form predominates. In the present disclosure, all tautomeric forms of the compound are included.

The term “stereoisomer” refers to isomers resulting from different spatial arrangements of atoms in a molecule, including cis-trans isomers, enantiomers, and diastereoisomers.

The compound of the present disclosure may have an asymmetric atom such as a carbon atom, a sulfur atom, a nitrogen atom, and a phosphorus atom, or an asymmetric double bond, and thus the compound of the present disclosure may exist in the form of a particular geometric isomer or stereoisomer. The form of a particular geometric isomer or stereoisomer may be cis and trans isomers, E and Z geometric isomers, (−)- and (+)-enantiomers, (R)- and (S)-enantiomers, diastereoisomers, (D)-isomers, (L)-isomers, and racemic mixtures or other mixtures thereof, such as an enantiomer or diastereoisomer enriched mixture, and all of the above isomers, as well as mixtures thereof, are encompassed within the definition scope of the compound of the present disclosure. An additional asymmetric carbon atom, asymmetric sulfur atom, asymmetric nitrogen atom, or asymmetric phosphorus atom may be present in substituents such as alkyl. All of these isomers and mixtures thereof referred to in the substituents are also encompassed within the definition scope of the compound of the present disclosure. The compound with asymmetric atoms of the present disclosure can be separated in an optically active pure form or in a racemic form. The optically active pure form can be obtained by resolving a racemic mixture or by synthesis using chiral starting materials or chiral reagents.

The term “substituted” means that any one or more hydrogen atoms on a specific atom are substituted with substituents, as long as the valence of the specific atom is normal and the compound resulting from the substitution is stable. When the substituent is oxo (namely ═O), it means that two hydrogen atoms are substituted, and oxo is not available on an aromatic group.

The term “optional” or “optionally” means that the subsequently described event or circumstance may, but not necessarily, occur. The description includes instances where the event or circumstance occurs and instances where the event or circumstance does not. For example, ethyl being “optionally” substituted with halogen means that the ethyl may be unsubstituted (CH₂CH₃), monosubstituted (CH₂CH₂F, CH₂CH₂Cl, or the like), polysubstituted (CHFCH₂F, CH₂CHF₂, CHFCH₂Cl, CH₂CHCl₂, or the like), or fully substituted (CF₂CF₃, CF₂CCl₃, CCl₂CCl₃, or the like). It will be understood by those skilled in the art that for any group comprising one or more substituents, no substitution or substituting pattern that is spatially impossible and/or cannot be synthesized will be introduced.

When any variable (e.g., R^{a} or R^{b}) occurs more than once in the constitution or structure of a compound, the variable is independently defined in each case. For example, if a group is substituted with 2 R^{b}, the definition of each R^{b} is independent.

When the number of a linking group is 0, such as —(CH₂)₀—, it means that the linking group is a bond.

When one of variables is selected from a chemical bond or is absent, it means that the two groups which it links are linked directly. For example, when L in A-L-Z represents a bond, it means that the structure is actually A-Z.

When the linking direction of the linking group referred to herein is not specified, the linking direction is arbitrary. For example, when L¹ in the structural unit

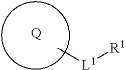

is selected from “C₁-C₃ alkylene-O”, then L¹ may link ring Q and R¹ in a direction from left to right to constitute “ring Q-C₁-C₃ alkylene-O—R¹”, or link ring Q and R¹ in a direction from right to left to constitute “ring Q-O—C₁-C₃ alkylene-R¹”.

Cₘ-Cₙ used herein means that the portion has an integer number of carbon atoms in the range of m-n. For example, “C₁-C₁₀” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “alkyl” refers to a hydrocarbon group with a general formula of CₙH₂ₙ₊₁. The alkyl may be linear or branched. The term “C₁₋₁₀ alkyl” may be understood to represent a linear or branched saturated hydrocarbon group having 1, 2, 3, 4, 5, 6, 7, 8, 9, or 10 carbon atoms. Specific examples of the alkyl include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, or the like. The term “C₁₋₆ alkyl” may be understood to represent an alkyl group having 1 to 6 carbon atoms. Specific examples include, but are not limited to, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like. The term “C₁-C₃ alkyl” may be understood to represent a linear or branched saturated alkyl group having 1 to 3 carbon atoms. The “C₁-C₁₀ alkyl” may include the range of “C₁-C₆ alkyl”, “C₁-C₃ alkyl”, or the like, and the “C₁-C₆ alkyl” may further include “C₁-C₃ alkyl”.

The term “alkoxy” refers to a group derived from a linear or branched alcohol by loss of a hydrogen atom from hydroxy, and may be understood as “alkyloxy” or “alkyl-O—”. The term “C₁-C₁₀ alkoxy” may be understood as “C₁-C₁₀ alkyloxy” or “C₁-C₁₀ alkyl-O—”; the term “C₁-C₆ alkoxy” may be understood as “C₁-C₆ alkyloxy” or “C₁-C₆ alkyl-O—”. The “C₁-C₁₀ alkoxy” may include the ranges of “C₁-C₆ alkoxy”, “C₁-C₃ alkoxy”, and the like, and the “C₁-C₆ alkoxy” may further include “C₁-C₃ alkoxy”.

The term “cycloalkyl” refers to a fully saturated carbon ring group that exists in the form of a monocyclic ring, a fused ring, a bridged ring, a spiro ring, or the like. Unless otherwise indicated, the carbon ring is generally a 3- to 20-membered ring. The term “C₃-C₁₂ cycloalkyl” refers to a cycloalkyl group having 3, 4, 5, 6, 7, 8, 9, 10, 11, or 12 ring carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo[2.2.1]heptyl), bicyclo[2.2.2]octyl, adamantyl, spiro[4.5]decyl, and the like. The term “C₃-C₁₂ cycloalkyl” may include the ranges of “C₅-C₁₂ cycloalkyl”, “C₃-C₁₀ cycloalkyl”, “C₆-C₁₀ cycloalkyl”, “C₃-C₆ cycloalkyl”, and the like. The term “C₅-C₁₂ cycloalkyl” refers to a cycloalkyl group having 5, 6, 7, 8, 9, 10, 11, or 12 ring carbon atoms. The term “C₃-C₁₀ cycloalkyl” refers to a cycloalkyl group having 3, 4, 5, 6, 7, 8, 9, or 10 ring carbon atoms. The term “C₆-C₁₀ cycloalkyl” refers to a cycloalkyl group having 6, 7, 8, 9, or 10 ring carbon atoms. The term “C₃-C₆ cycloalkyl” refers to a cycloalkyl group having 3, 4, 5, or 6 ring carbon atoms.

The term “C₃-C₆ cycloalkyloxy” may be understood as “C₃-C₆ cycloalkyl-O—”.

The term “cycloalkenyl” refers to anon-aromatic carbon ring group that is not fully saturated, has at least one carbon-carbon double bond, and exists in the form of a monocyclic ring, a fused ring, a bridged ring, a spiro ring, or the like. The term “C₄-C₁₂ cycloalkenyl” refers to a cycloalkenyl group with 4, 5, 6, 7, 8, 9, 10, 11, 12, 13, or 14 ring atoms. Specific examples include, but are not limited to, cyclopentenyl, cyclopentadienyl, cyclohexenyl, cyclohexadienyl, cycloheptenyl, cycloheptadienyl, or the like.

The term “heterocyclyl” refers to a fully saturated or partially saturated monocyclic, fused cyclic, spiro cyclic, or bridged cyclic group, and ring atoms of the group include 1-5 heteroatoms or heteroatom groups (i.e., heteroatom-containing atom groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —P(═O)₂—, —P(═O)—, —NH—, —S(═O)(═NH)—, —C(═O)NH—, —NHC(═O)NH—, or the like. The term “4- to 14-membered heterocyclyl” refers to a heterocyclyl group with 4, 5, 6, 7, 8, 9, 10, 11, 12, 13, or 14 ring atoms, and ring atoms of the group include 1-5 heteroatoms or heteroatom groups independently selected from those described above. Specific examples of 4-membered heterocyclyl include, but are not limited to, azetidinyl or oxetanyl; specific examples of 5-membered heterocyclyl include, but are not limited to, tetrahydrofuranyl, dioxolyl, pyrrolidinyl, imidazolidinyl, pyrazolidinyl, pyrrolinyl, 4,5-dihydrooxazolyl, or 2,5-dihydro-1H-pyrrolyl; specific examples of 6-membered heterocyclyl include, but are not limited to, tetrahydropyranyl, piperidinyl, morpholinyl, dithianyl, thiomorpholinyl, piperazinyl, trithianyl, tetrahydropyridyl, or 4H-[1,3,4]thiadiazinyl; and specific examples of 7-membered heterocyclyl include, but are not limited to, diazepanyl. The heterocyclyl may also be a bicyclic heterocyclyl group or a tricyclic heterocyclyl group, wherein specific examples of 5,5-membered bicyclic groups include, but are not limited to, hexahydrocyclopenta[c]pyrrol-2(1H)-yl; and specific examples of 5,6-membered bicyclic groups include, but are not limited to, hexahydropyrrolo[1,2-a]pyrazin-2(1H)-yl, 5,6,7,8-tetrahydro-[1,2,4]triazolo[4,3-a]pyrazinyl, or 5,6,7,8-tetrahydroimidazo[1,5-a]pyrazinyl. Optionally, the heterocyclyl may be a benzo-fused ring group of the 4- to 7-membered heterocyclyl described above. Specific examples include, but are not limited to, dihydroisoquinolyl and the like. The “4- to 14-membered heterocyclyl” may include the range of “4- to 7-membered heterocyclyl”, “5- to 6-membered heterocyclyl”, “4- to 7-membered monocyclic heterocyclyl”, “6- to 14-membered fused heterocyclyl”, “6- to 14-membered spiro heterocyclyl”, “6- to 14-membered bridged heterocyclyl”, or the like. The term “6- to 14-membered fused heterocyclyl” refers to a fused heterocyclyl group with 6, 7, 8, 9, 10, 11, 12, 13, or 14 ring atoms, and ring atoms of the group include 1-5 heteroatoms or heteroatom groups independently selected from those described above. The term “6- to 14-membered spiro heterocyclyl” refers to a spiro heterocyclyl group with 6, 7, 8, 9, 10, 11, 12, 13, or 14 ring atoms, and ring atoms of the group include 1-5 heteroatoms or heteroatom groups independently selected from those described above. The term “6- to 14-membered bridged heterocyclyl” refers to a bridged heterocyclyl group with 6, 7, 8, 9, 10, 11, 12, 13, or 14 ring atoms, and ring atoms of the group include 1-5 heteroatoms or heteroatom groups independently selected from those described above.

The “heterocyclyl” herein may include “heterocycloalkyl”, for example, the “4- to 14-membered heterocyclyl” may include “4- to 14-membered heterocycloalkyl”.

The term “heterocyclyloxy” may be understood as “heterocyclyl-O—”.

The term “heterocycloalkyl” refers to a fully saturated cyclic group that exists in the form of a monocycle, fused cycle, bridged cycle, spiro cycle, or the like, and ring atoms of the group include 1-5 heteroatoms or heteroatom groups (i.e., heteroatom-containing atom groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —NH—, —S(═O)(═NH)—, —C(═O)NH—, —NHC(═O)NH—, or the like. The term “3- to 10-membered heterocycloalkyl” refers to a heterocycloalkyl group with 3, 4, 5, 6, 7, 8, 9, or 10 ring atoms, and ring atoms of the group include 1-5 heteroatoms or heteroatom groups independently selected from those described above. The term “3- to 10-membered heterocycloalkyl” may include “4- to 10-membered heterocycloalkyl” or “4- to 7-membered heterocycloalkyl”, wherein specific examples of 4-membered heterocycloalkyl include, but are not limited to, azetidinyl, oxetanyl, or thietanyl; specific examples of 5-membered heterocycloalkyl include, but are not limited to, tetrahydrofuranyl, tetrahydrothienyl, pyrrolidinyl, isoxazolidinyl, oxazolidinyl, isothiazolidinyl, thiazolidinyl, imidazolidinyl, or tetrahydropyrazolyl; specific examples of 6-membered heterocycloalkyl include, but are not limited to, piperidinyl, tetrahydropyranyl, tetrahydrothiopyranyl, morpholinyl, piperazinyl, 1,4-thioxanyl, 1,4-dioxanyl, thiomorpholinyl, 1,3-dithianyl, or 1,4-dithianyl; and specific examples of 7-membered heterocycloalkyl include, but are not limited to, azepanyl, oxepanyl, or thiepanyl.

The term “heterocycloalkyloxy” may be understood as “heterocycloalkyl-O—”.

The term “aryl” refers to an aromatic all-carbon monocyclic or fused polycyclic group with a conjugated π-electron system. Aryl may have 6-20 carbon atoms, 6-14 carbon atoms, or 6-12 carbon atoms. The term “C₆-C₂₀ aryl” may be understood as an aryl group having 6-20 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl, or a ring having 13 carbon atoms (“C₁₃ aryl”), such as fluorenyl, or a ring having 14 carbon atoms (“C₁₄ aryl”), such as anthryl. The term “C₆-C₁₀ aryl” may be understood as an aryl group having 6-10 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl.

The term “aryloxy” may be understood as “aryl-O—”.

The term “heteroaryl” refers to an aromatic cyclic group having an aromatic monocyclic or fused polycyclic system, which contains at least one ring atom selected from N, O, and S, with the remaining ring atoms being C. The term “5- to 10-membered heteroaryl” may be understood to include an aromatic monocyclic or bicyclic ring system, which has 5, 6, 7, 8, 9, or 10 ring atoms, particularly 5, 6, 9, or 10 ring atoms, and comprises 1-5, preferably 1-3, heteroatoms independently selected from N, O, and S. In particular, the heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, and the like, and benzo derivatives thereof, such as benzofuranyl, benzothienyl, benzothiazolyl, benzoxazolyl, benzoisoxazolyl, benzimidazolyl, benzotriazolyl, indazolyl, indolyl, isoindolyl, and the like; and pyridyl, pyridazinyl, pyrimidinyl, pyrazinyl, triazinyl, and the like, and benzo derivatives thereof, such as quinolyl, quinazolinyl, isoquinolyl, and the like; and azocinyl, indolizinyl, purinyl, and the like, and benzo derivatives thereof; and cinnolinyl, phthalazinyl, quinazolinyl, quinoxalinyl, naphthyridinyl, pteridinyl, carbazolyl, acridinyl, phenazinyl, phenothiazinyl, phenoxazinyl, and the like.

The term “heteroaryloxy” may be understood as “heteroaryl-O—”.

The term “halo” or “halogen” refers to fluorine, chlorine, bromine, or iodine.

The term “hydroxy” refers to the —OH group.

The term “cyano” refers to the —CN group.

The term “sulfydryl” refers to the —SH group.

The term “amino” refers to the —NH₂ group.

The term “nitro” refers to the —NO₂ group.

The term “therapeutically effective amount” refers to an amount of the compound of the present disclosure for (i) treating a specific disease, condition or disorder; (ii) alleviating, ameliorating or eliminating one or more symptoms of a specific disease, condition or disorder, or (iii) delaying the onset of the one or more symptoms of the specific disease, condition or disorder described herein. The amount of the compound disclosed herein constituting the “therapeutically effective amount” varies dependently on the compound, the disease state and its severity, the administration regimen, and the age of the mammal to be treated, but can be determined routinely by those skilled in the art in accordance with their knowledge and the present disclosure.

The term “prevent” or “prevention” means administering the compound or formulation described herein to prevent a disease or one or more symptoms associated with the disease, and includes: preventing the occurrence of the disease or disease state in an individual (e.g., a mammal), particularly when such an individual (e.g., a mammal) is predisposed to the disease state but has not yet been diagnosed with it.

The term “individual” includes mammals and non-mammals. Examples of mammals include, but are not limited to, any member of the class Mammalia: humans, non-human primates (e.g., chimpanzees and other apes and monkeys); livestock animals, such as cattle, horses, sheep, goats, and pigs; domestic animals, such as rabbits, dogs, and cats; laboratory animals, including rodents, such as rats, mice, guinea pigs, and the like. Examples of non-human mammals include, but are not limited to, birds, fish, and the like. In one embodiment associated with the methods and compositions provided herein, the mammal is a human. The terms “patient” and “individual” are used interchangeably.

The term “pharmaceutically acceptable” is used herein for those compounds, materials, compositions, and/or dosage forms which are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to salts of pharmaceutically acceptable acids or bases, including salts formed from the compound and an inorganic or organic acid, and salts formed from the compound and an inorganic or organic base.

The term “pharmaceutical composition” refers to a mixture consisting of one or more of the compounds or the salts thereof of the present disclosure and a pharmaceutically acceptable excipient. The pharmaceutical composition is intended to facilitate the administration of the compound of the present disclosure to an organism.

The term “pharmaceutically acceptable excipients” refers to those that do not have a significant irritating effect on an organism and do not impair the biological activity and properties of the active compound. Suitable excipients are well known to those skilled in the art, such as carbohydrate, wax, water-soluble and/or water-swellable polymers, hydrophilic or hydrophobic materials, gelatin, oil, solvent, water, and the like.

The word “comprise” and variations thereof such as “comprises” or “comprising” may be understood in an open, non-exclusive sense, i.e., “including but not limited to”.

The present disclosure also includes isotopically labeled compounds of the present disclosure which are identical to those documented herein but have one or more atoms replaced by an atom having an atomic mass or mass number different from the atomic mass or mass number usually found in nature. Examples of isotopes that can be incorporated into the compounds of the present disclosure include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N, ¹⁵O, ¹⁷O, ¹⁸O ³¹P, ³²P, ³⁵S, ¹⁸F, ¹²³I, ¹²⁵I and ³⁶Cl, respectively, and the like.

Certain isotopically labeled compounds of the present disclosure (e.g., those labeled with ³H and ¹⁴C) can be used to analyze compounds and/or substrate tissue distribution. Tritiated (i.e., ³H) and carbon-14 (i.e., ¹⁴C) isotopes are particularly preferred for their ease of preparation and detectability. Positron emitting isotopes, such as ¹⁵O, ¹³N, ¹¹C, and ¹⁸F, can be used in positron emission tomography (PET) studies to determine substrate occupancy. Isotopically labeled compounds of the present disclosure can generally be prepared by following procedures analogous to those disclosed in the schemes and/or examples below while substituting a non-isotopically labeled reagent with an isotopically labeled reagent.

The pharmaceutical composition of the present disclosure can be prepared by combining the compound of the present disclosure with a suitable pharmaceutically acceptable excipient, and can be formulated, for example, into a solid, semisolid, liquid or gaseous formulation such as tablet, pill, capsule, powder, granule, ointment, emulsion, suspension, suppository, injection, inhalant, gel, microsphere, aerosol, and the like.

Typical routes of administration of the compound or the pharmaceutically acceptable salt thereof or the pharmaceutical composition thereof of the present disclosure include, but are not limited to, oral, rectal, local, inhalation, parenteral, sublingual, intravaginal, intranasal, intraocular, intraperitoneal, intramuscular, subcutaneous and intravenous administration.

The pharmaceutical composition of the present disclosure can be manufactured by methods well known in the art, such as conventional methods of mixing, dissolving, granulating, emulsifying, lyophilizing, and the like.

In some embodiments, the pharmaceutical composition is in an oral form. For oral administration, the pharmaceutical composition can be formulated by mixing the active compounds with pharmaceutically acceptable excipients well known in the art. These excipients enable the compounds of the present disclosure to be formulated into tablets, pills, lozenges, dragees, capsules, liquids, gels, slurries, suspensions, and the like, for oral administration to patients.

A solid oral composition can be prepared by conventional mixing, filling or tableting. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to: binders, diluents, disintegrants, lubricants, glidants, or flavoring agents, and the like.

The pharmaceutical compositions may also be suitable for parenteral administration, such as sterile solutions, suspensions or lyophilized products in suitable unit dosage forms.

In all of the administration methods for the compound of general formula I described herein, the daily dose administered is from 0.01 mg/kg body weight to 200 mg/kg body weight, preferably from 0.05 mg/kg body weight to 50 mg/kg body weight, and more preferably from 0.1 mg/kg body weight to 30 mg/kg body weight, given in a single dose or divided doses.

The compounds disclosed herein can be prepared by a variety of synthetic methods well known to those skilled in the art, including the specific embodiments listed below, embodiments formed by combinations thereof with other chemical synthetic methods, and equivalents thereof well known to those skilled in the art. The preferred embodiments include, but are not limited to, the examples disclosed herein.

The chemical reactions of the specific embodiments disclosed herein are conducted in a suitable solvent that must be suitable for the chemical changes in the present disclosure and the reagents and materials required. In order to obtain the compounds disclosed herein, it is sometimes necessary for those skilled in the art to modify or select a synthesis procedure or a reaction process based on the existing embodiments.

### DETAILED DESCRIPTION

The present disclosure is described in detail below by way of examples, which, however, are not intended to disadvantageously limit the scope of the present disclosure in any way. Although the present disclosure has been described in detail herein and specific embodiments thereof have also been disclosed, it will be apparent to those skilled in the art that various changes and modifications can be made to the specific embodiments of the present disclosure without departing from the spirit and scope of the present disclosure. All reagents used in the present disclosure are commercially available and can be used without further purification.

Unless otherwise stated, the ratios expressed for mixed solvents are volume mixing ratios.

Unless otherwise stated,% refers to weight percentage wt %.

Compounds are named either manually or by ChemDraw® software, and supplier's catalog names are given for commercially available compounds.

The structures of the compounds are determined by nuclear magnetic resonance (NMR) and/or mass spectrometry (MS). NMR shifts are given in 10⁻⁶ (ppm). The solvents for NMR determination are deuterated dimethyl sulfoxide, deuterated chloroform, deuterated methanol, and the like, and the internal standard is tetramethylsilane (TMS); “IC₅₀” refers to the half maximal inhibitory concentration, which is the concentration at which half of the maximal inhibitory effect is achieved.

The eluent or mobile phase may be a mixed eluent or mobile phase composed of two or more solvents, and the ratio of the mixed eluent or mobile phase is the volume ratio of the solvents. For example, “0-10% methanol/dichloromethane” represents that the volume ratio of methanol to dichloromethane in the mixed eluent or mobile phase is 0:100-10:100.

### Example 1: Preparation of (1R,2S,5S)—N-((1S)-1-cyano-2-(2-carbonylindolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 1)

The synthetic route was as follows:

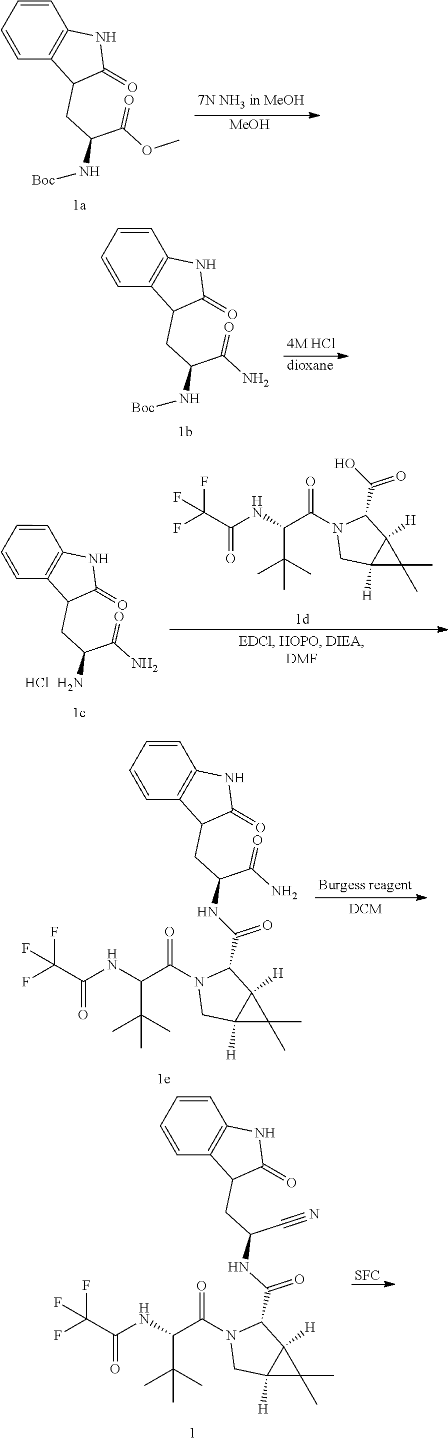

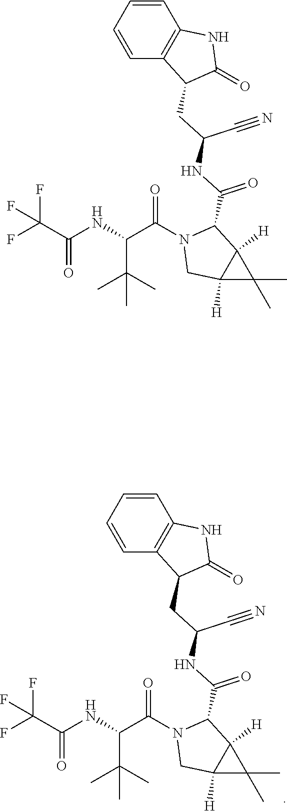 [SMILES: CC(C)(C)[C@H](NC(=O)C(F)(F)F)C(=O)N1C[C@H]2[C@@H]([C@H]1C(=O)N[C@H](C#N)C[C@H]1C(=O)Nc3ccccc31)C2(C)C ; CC(C)(C)[C@H](NC(=O)C(F)(F)F)C(=O)N1C[C@H]2[C@@H]([C@H]1C(=O)N[C@H](C#N)C[C@@H]1C(=O)Nc3ccccc31)C2(C)C]

### Step 1: tert-butyl ((2S)-1-amino-1-oxo-3-(2-oxoindol-3-yl)propan-2-yl)carbamate (1b)

At 20° C., compound 1a (0.30 g, 0.90 mmol) was dissolved in methanol (1 mL), and a solution of ammonia in methanol (7 N, 3 mL) was added. The reaction liquid was allowed to react at 40° C. for 24 h. Then, the reaction liquid was distilled under reduced pressure to remove the solvent. The resulting residue was purified by reverse-phase chromatography (SepaFlash® C₁₈ flash silica gel column, gradient elution with 0.05% aqueous formic acid solution:acetonitrile=20:1-1:2) to obtain compound 1b (0.09 g).

m/z (ESI): 320 [M+H]⁺.

Step 2: (2S)-2-amino-3-(2-carbonylindolin-3-yl)propanamide hydrochloride (1c)

At 20° C., compound 1b (0.090 g, 0.28 mmol) was dissolved in 1,4-dioxane (1 mL), and a solution of hydrochloric acid in 1,4-dioxane (4 M, 1 mL, 4.0 mmol) was added. The reaction liquid was allowed to react at room temperature for 2 h. Then, the reaction liquid was lyophilized to obtain compound 1c (0.070 g).

m/z (ESI): 220 [M+H]⁺.

### Step 3: (1R,2S,5S)—N-((2S)-1-amino-1-carbonyl-3-(2-carbonylindolin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (1e)

At 20° C., 2-hydroxypyridine-1-oxide (0.005 g, 0.082 mmol) was added to a solution of compound 1d (0.10 g, 0.27 mmol) and compound 1c (0.07 g, 0.27 mmol) in N,N-dimethylformamide (2 mL). Then, N,N-diisopropylethylamine (0.11 g, 0.82 mmol) and 1-ethyl-(3-dimethylaminopropyl)carbodiimide hydrochloride (0.079 g, 0.41 mmol) were added sequentially. The reaction liquid was allowed to react at 25° C. for 12 h. Then, the reaction mixture was purified by reverse-phase chromatography (SepaFlash® Cis flash silica gel column, gradient elution with 0.05% aqueous formic acid solution:acetonitrile=20:1-1:1) to obtain compound 1e (0.04 g).

m/z (ESI): 566 [M+H]⁺.

### Step 4: (1R,2S,5S)—N-((1S)-1-cyano-2-(2-carbonylindolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (1)

At 20° C., methyl N-(triethylammoniumsulfonyl)carbamate inner salt (0.025 g, 0.11 mmol) was added to a solution of compound 1e (0.02 g, 0.035 mmol) in dichloromethane (1 mL). The reaction mixture was allowed to react at 25° C. for 12 h. Then, the reaction liquid was concentrated under reduced pressure. The resulting residue was purified by reverse-phase chromatography (SepaFlash® Cis flash silica gel column, gradient elution with 0.05% aqueous formic acid solution:acetonitrile=20:1-1:2) to obtain 1 mg of the first isomer product (compound 1-Pt) and 7 mg of the second isomer product (compound 1-P2) in sequence.

The retention time (RT) of compound 1-P2 under the following high-performance liquid chromatography conditions was 1.64 min.

High-performance liquid chromatography conditions: The column was Xtimate C18 2.1×50 mm; mobile phase A was 0.05% trifluoroacetic acid in water; mobile phase B was 0.05% trifluoroacetic acid in acetonitrile; the flow rate was 0.7 mL/min; and B % was 5%-95%.

The structural characterization data for compound 1-P2 are as follows:

m/z (ESI): 548 [M+H]⁺.

¹H NMR: (400 MHz, DMSO-d6) δ 10.58-10.41 (m, 1H), 9.42-9.00 (m, 2H), 7.39-7.26 (m, 1H), 7.21-7.13 (m, 1H), 7.03-6.78 (m, 2H), 5.30-5.16 (m, 1H), 4.44-4.30 (m, 1H), 4.22 (s, 1H), 4.00-3.86 (m, 1H), 3.75-3.64 (m, 1H), 3.58-3.47 (m, 1H), 2.19-2.05 (m, 1H), 1.63-1.54 (m, 1H), 1.35 (d, J=7.7 Hz, 1H), 1.04 (s, 3H), 1.01-0.96 (m, 2H), 0.94 (s, 2H), 0.90-0.79 (m, 9H).

### Example 2: Preparation of (1R,2S,5S)-3-((S)-2-(adamantan-1-yl)-2-(2,2,2-trifluoroacetamido)acetyl)-N—((S)-1-cyano-2-((S)-2-carbonylpyrrolidin-3-yl)ethyl)-6,6-dimethyl-3-azabicyclo[3.1.0]hexane-2-carboxamide (Compound 2)

The synthetic route was as follows:

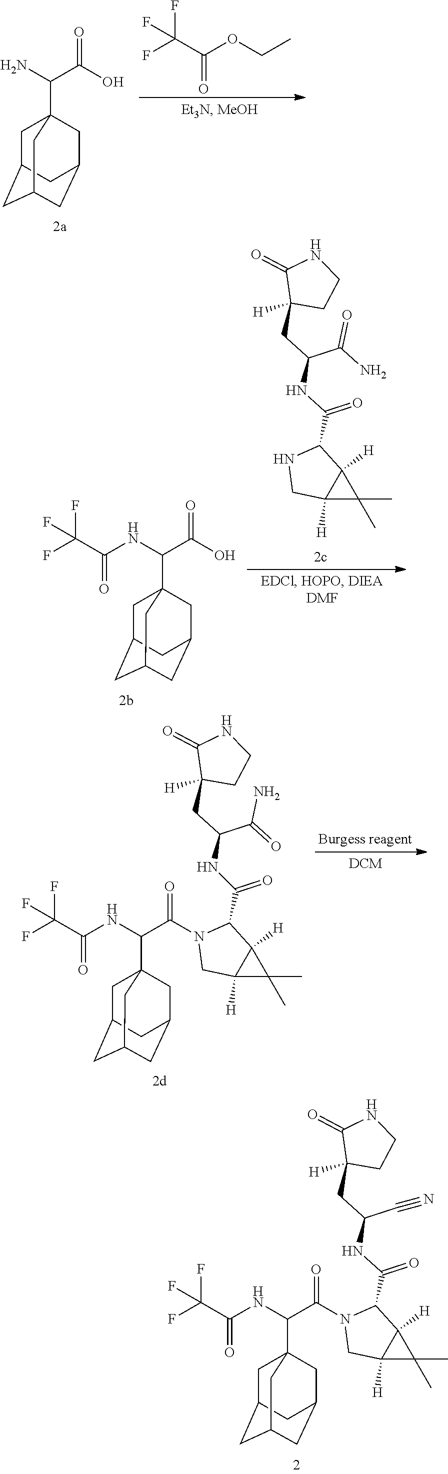

### Step 1: (S)-2-(adamantan-1-yl)-2-(2,2,2-trifluoroacetamido)acetic acid (2b)

At 20° C., compound 2a (0.15 g, 0.72 mmol) was dissolved in methanol (10 mL), and then triethylamine (0.46 g, 3.6 mmol) and ethyl trifluoroacetate (0.51 g, 3.6 mmol) were added. The reaction liquid was heated to 50° C. and allowed to react for 16 h. Then, the reaction liquid was concentrated, adjusted to pH 3-4 by adding 1 M hydrochloric acid, and extracted with ethyl acetate (3×10 mL). The organic phase was dried over anhydrous sodium sulfate, filtered, and concentrated to obtain compound 2b (0.2 g). m/z (ESI): 306 [M+H]⁺.

### Step 2: (1R,2S,5S)-3-((S)-2-(adamantan-1-yl)-2-(2,2,2-trifluoroacetamido)acetyl)-N—((S)-1-amino-1-carbonyl-3-((S)-2-carbonylpyrrolidin-3-yl)propan-2-yl)-6,6-dimethyl-3-azabicyclo[3.1.0]hexane-2-carboxamide (2d)

At 20° C., 2-hydroxypyridine-1-oxide (0.004 g, 0.033 mmol) was added to a solution of compound 2b (0.1 g, 0.33 mmol) and compound 2c (0.11 g, 0.33 mmol) in N,N-dimethylformamide (2 mL), and then N,N-diisopropylethylamine (0.13 g, 0.98 mmol) and 1-ethyl-(3-dimethylaminopropyl)carbodiimide hydrochloride (0.094 g, 0.49 mmol) were added. The reaction liquid was allowed to react at 20° C. for 12 h. Then, the reaction mixture was purified by reverse-phase chromatography (SepaFlash® Cis flash silica gel column, gradient elution with 0.05% aqueous formic acid solution:acetonitrile=20:1-1:1) to obtain compound 2d (0.018 g).

m/z (ESI): 596 [M+H]⁺.

### Step 3: (1R,2S,5S)-3-((S)-2-(adamantan-1-yl)-2-(2,2,2-trifluoroacetamido)acetyl)-N—((S)-1-cyano-2-((S)-2-carbonylpyrrolidin-3-yl)ethyl)-6,6-dimethyl-3-azabicyclo[3.1.0]hexane-2-carboxamide (2)

At 20° C., methyl-N-(triethylammoniumsulfonyl)carbamate inner salt (0.023 g, 0.091 mmol) was added to a solution of compound 2d (0.018 g, 0.03 mmol) in dichloromethane (1 mL). The reaction liquid was allowed to react at 20° C. for 12 h. Then, the reaction liquid was concentrated under reduced pressure. The resulting residue was purified by reverse-phase chromatography (SepaFlash® C₁₈ flash silica gel column, gradient elution with 0.05% aqueous formic acid solution:acetonitrile=20:1-1:2) to obtain compound 2 (0.004 g).

m/z (ESI): 578 [M+H]⁺.

¹HNMR: (400 MHz, Chloroform-d) δ 9.59 (d, J=3.6 Hz, 0.4H), 8.17 (d, J=6.6 Hz, 0.6H), 6.94 (d, J=9.3 Hz, 0.4H), 6.89 (d, J=9.4 Hz, 0.6H), 5.72 (s, 1H), 4.89 (dt, J=10.3, 6.2 Hz, 0.6H), 4.58 (dt, J=12.1, 4.0 Hz, 0.4H), 4.41 (d, J=9.4 Hz, 0.6H), 4.28 (s, 0.6H), 4.26 (s, 0.4H), 4.03-3.96 (m, 0.6H), 3.92-3.84 (m, 0.8H), 3.78 (d, J=10.3 Hz, 0.6H), 3.54 (d, J=12.4 Hz, 0.4H), 3.46-3.33 (m, 2H), 3.26-3.16 (m, 0.4H), 2.63-2.51 (m, 1H), 2.49-2.14 (m, 2H), 2.07-1.88 (m, 5H), 1.73-1.64 (m, 6H), 1.60-1.51 (m, 6H), 1.42-1.25 (m, 2H), 1.07 (s, 1.2H), 1.06 (s, 1.8H), 1.03 (s, 1.2H), 0.86 (s, 1.8H).

### Example 3: Preparation of (1R,2S,5S)—N—((S)-1-cyano-2-((S)-2-carbonylpyrrolidin-3-yl)ethyl)-6,6-dimethyl-3-(2-(spiro[3.3]heptan-2-yl)-2-(2,2,2-trifluoroacetamido)acetyl)-3-azabicyclo[3.1.0]hexane-2-carboxamide (Compound 3)

The synthetic route was as follows:

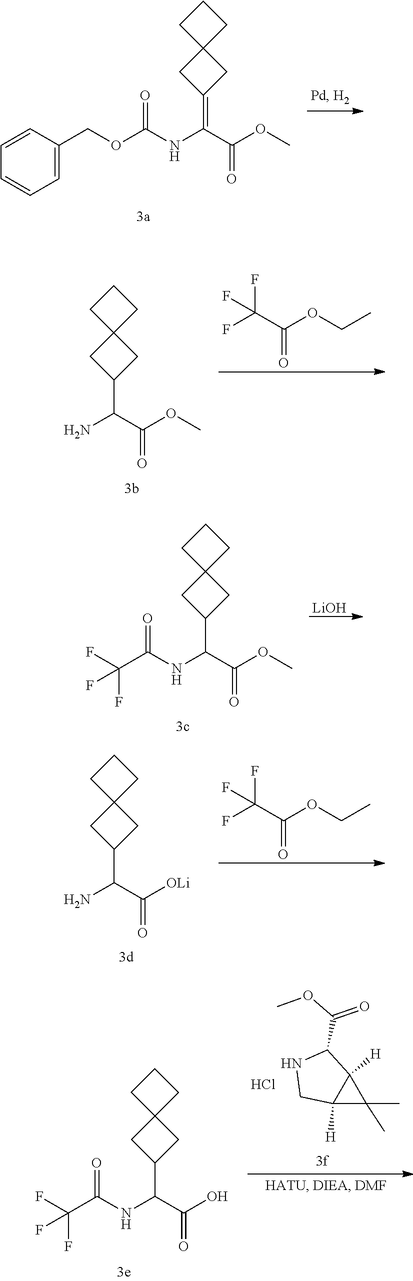

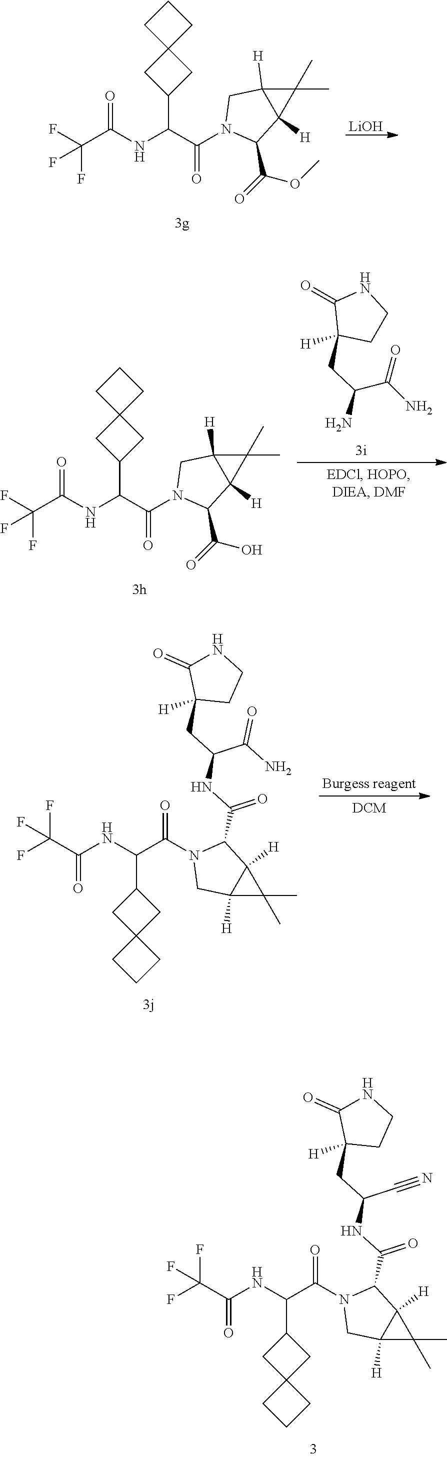

### Step 1: methyl 2-amino-2-(spiro[3.3]heptan-2-yl)acetate (3b)

Under a hydrogen atmosphere, compound 3a (1.6 g, 5.1 mmol) was dissolved in ethyl acetate (100 mL), and then palladium on carbon (0.32 g) was added. The reaction liquid was allowed to react at 20° C. for 16 h. Then, the reaction liquid was filtered and distilled under reduced pressure to remove the solvent to obtain compound 3b (0.81 g).

m/z (ESI): 184 [M+H]⁺.

### Step 2: methyl 2-(spiro[3.3]heptan-2-yl)-2-(2,2,2-trifluoroacetamido)acetate (3c)

At 20° C., compound 3b (0.81 g, 4.41 mmol) was dissolved in methanol (20 mL), and then triethylamine (1.3 g, 13 mmol) and ethyl trifluoroacetate (3.1 g, 22 mmol) were added. The mixture was then heated to 50° C. and allowed to react for 16 h. Then, the reaction liquid was concentrated, adjusted to pH 3-4 by adding 1 M hydrochloric acid, and extracted with ethyl acetate (3×50 mL). The organic phase was dried over anhydrous sodium sulfate, filtered, and concentrated to obtain compound 3c (0.97 g).

m/z (ESI): 280 [M+H]⁺.

### Step 3: lithium 2-amino-2-(spiro[3.3]heptan-2-yl)acetate (3d)

Compound 3c (0.97 g, 3.5 mmol) was dissolved in tetrahydrofuran (5 mL) and water (5 mL), and lithium hydroxide (0.33 g, 14 mmol) was added. The reaction liquid was allowed to react at 20° C. for 2 h. Then, ethyl acetate (30 mL) was added, and the mixture was filtered to obtain compound 3d (0.56 g).

### Step 4: 2-(spiro[3.3]heptan-2-yl)-2-(2,2,2-trifluoroacetamido)acetic acid (3e)

At room temperature, lithium 2-amino-2-(spiro[3.3]heptan-2-yl)acetate (0.56 g, 3.3 mmol) was dissolved in methanol (20 mL), and then triethylamine (1.0 g, 9.9 mmol) and ethyl trifluoroacetate (2.3 g, 16 mmol) were added. The mixture was then heated to 50° C. and allowed to react for 16 h. Then, the reaction liquid was concentrated, adjusted to pH 3-4 by adding 1 M hydrochloric acid, and extracted with ethyl acetate (3×50 mL). The organic phase was dried over anhydrous sodium sulfate, filtered, and concentrated to obtain compound 3e (0.56 g).

m/z (ESI): 266 [M+H]⁺.

### Step 5: methyl (1R,2S,5S)-6,6-dimethyl-3-(2-(spiro[3.3]heptan-2-yl)-2-(2,2,2-trifluoroacetamido)acetyl)-3-azabicyclo[3.1.0]hexane-2-carboxylate (3g)

At 0° C., compound 3f (0.15 g, 0.75 mmol) and compound 3e (0.2 g, 0.75 mmol) were dissolved in N,N-dimethylformamide (3 mL), and O-(7-azabenzotriazol-1-yl)-N,N,N′,N′-tetramethyluronium hexafluorophosphate (0.43 g, 1.1 mmol) was added, followed by the addition of N,N-diisopropylethylamine (0.23 g, 2.3 mmol). The reaction liquid was allowed to react at 20° C. for 2 h. Then, the reaction mixture was purified by reverse-phase chromatography (SepaFlash® Cis flash silica gel column, gradient elution with 0.05% aqueous formic acid solution:acetonitrile=20:1-1:2) to obtain compound 3g (0.23 g).

m/z (ESI): 417 [M+H]⁺.

### Step 6: (1R,2S,5S)-6,6-dimethyl-3-(2-(spiro[3.3]heptan-2-yl)-2-(2,2,2-trifluoroacetamido)acetyl)-3-azabicyclo[3.1.0]hexane-2-carboxylic acid (3h)

Compound 3g (0.23 g, 0.55 mmol) was dissolved in tetrahydrofuran (5 mL) and water (5 mL), and then lithium hydroxide (0.1 g, 2.2 mmol) was added. The reaction liquid was allowed to react at 20° C. for 2 h. Then, the reaction liquid was adjusted to pH=7 by adding 1 M hydrochloric acid and extracted with ethyl acetate (3×50 mL). The organic phase was dried over anhydrous sodium sulfate, filtered, and concentrated to obtain compound 3h (0.18 g).

m/z (ESI): 403 [M+H]⁺.

### Step 7: (1R,2S,5S)—N—((S)-1-amino-1-carbonyl-3-((S)-2-carbonylpyrrolidin-3-yl)propan-2-yl)-6,6-dimethyl-3-(2-(spiro[3.3]heptan-2-yl)-2-(2,2,2-trifluoroacetamido)acetyl)-3-azabicyclo[3.1.0]hexane-2-carboxamide (3j)

At 20° C., 2-hydroxypyridine-1-oxide (0.004 g, 0.041 mmol) was added to a solution of compound 3h (0.16 g, 0.41 mmol) and compound 3i (0.07 g, 0.41 mmol) in N,N-dimethylformamide (2 mL). Then, N,N-diisopropylethylamine (0.12 g, 1.2 mmol) and 1-ethyl-(3-dimethylaminopropyl)carbodiimide hydrochloride (0.096 g, 0.62 mmol) were added. The reaction liquid was allowed to react at 20° C. for 12 h. Then, the reaction mixture was purified by reverse-phase chromatography (SepaFlash® Cis flash silica gel column, gradient elution with 0.05% aqueous formic acid solution:acetonitrile=20:1-1:1) to obtain compound 3j (0.16 g).

m/z (ESI): 556 [M+H]⁺.

### Step 8: (1R,2S,5S)—N—((S)-1-cyano-2-((S)-2-carbonylpyrrolidin-3-yl)ethyl)-6,6-dimethyl-3-(2-(spiro[3.3]heptan-2-yl)-2-(2,2,2-trifluoroacetamido)acetyl)-3-azabicyclo[3.1.0]hexane-2-carboxamide (3)

At room temperature, methyl N-(triethylammoniumsulfonyl)carbamate inner salt (0.1 g, 0.43 mmol) was added to a solution of compound 3j (0.08 g, 0.14 mmol) in dichloromethane (5 mL). The reaction liquid was allowed to react at 20° C. for 12 h. Then, the reaction liquid was distilled under reduced pressure to remove the solvent. The resulting residue was purified by reverse-phase chromatography (SepaFlash® C₁₈ flash silica gel column, gradient elution with 0.05% aqueous formic acid solution:acetonitrile=20:1-1:2) to obtain compound 3 (0.060 g).

m/z (ESI): 538 [M+H]⁺.

¹HNMR: (400 MHz, DMSO-d6) δ 9.70 (d, J=6.1 Hz, 0.6H), 9.64 (d, J=8.3 Hz, 0.4H), 8.95 (d, J=8.4 Hz, 0.6H), 8.79 (d, J=8.3 Hz, 0.4H), 7.75-7.67 (m, 1H), 5.02-4.89 (m, 1H), 4.54-4.47 (m, 0.4H), 4.38-4.33 (m, 0.6H), 4.12 (s, 0.6H), 4.06 (s, 0.4H), 3.91 (d, J=5.6 Hz, 0.4H), 3.88 (d, J=5.5 Hz, 0.6H), 3.75 (d, J=10.3 Hz, 0.6H), 3.66 (d, J=11.1 Hz, 0.4H), 3.21-3.06 (m, 2H), 2.47-2.32 (m, 2H), 2.16-2.02 (m, 2H), 1.97-1.82 (m, 5H), 1.81-1.62 (m, 6H), 1.60-1.41 (m, 2H), 1.35-1.27 (m, 1H), 1.06-1.01 (m, 3H), 0.92 (s, 1.2H), 0.87 (s, 1.8H).

### Example 4: (1R,2S,5S)—N—((S)-1-Cyano-2-((S)-2-carbonylpyrrolidin-3-yl)ethyl)-6,6-dimethyl-3-((S)-3-(naphthalen-2-yl)-2-(2,2,2-trifluoroacetamido)propanoyl)-3-azabicyclo[3.1.0]hexane-2-carboxamide (Compound 4)

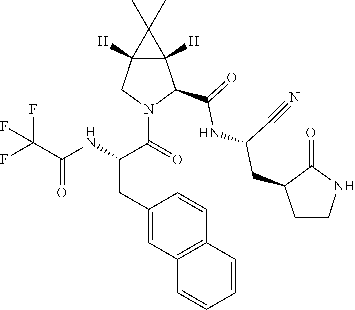 [SMILES: CC1(C)[C@@H]2[C@@H](C(=O)N[C@H](C#N)C[C@@H]3CCNC3=O)N(C(=O)[C@H](Cc3ccc4ccccc4c3)NC(=O)C(F)(F)F)C[C@@H]21]

Compound 4 was prepared by the same preparation method as in Example 2 except for replacing compound 2a with compound 4a.

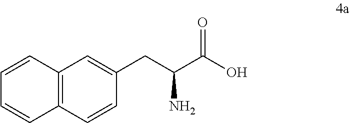 [SMILES: N[C@@H](Cc1ccc2ccccc2c1)C(=O)O]

m/z (ESI): 584 [M+H]⁺.

¹HNMR: (400 MHz, DMSO-d₆) δ 10.0 (d, J=7.2 Hz, 1H), 8.99 (d, J=8.0 Hz, 1H), 7.89-7.75 (m, 5H), 7.50-7.47 (m, 3H), 5.02-4.96 (m, 1H), 4.77-4.69 (m, 1H), 4.19 (s, 1H), 3.92-3.89 (m, 1H), 3.74 (d, J=10.8 Hz, 1H), 3.25-3.05 (m, 4H), 2.47-2.38 (m, 1H), 2.21-2.10 (m, 2H), 1.83-1.69 (m, 2H), 1.60-1.57 (m, 1H), 1.35 (d, J=8.0 Hz, 1H), 1.04 (s, 3H), 0.89 (s, 3H).

### Example 5: (1R,2S,5S)—N-((1S)-1-Cyano-2-(2-carbonylindolin-3-yl)ethyl)-3-((S)-2-((1r,3R,5R,7S)-3-hydroxyadamantan-1-yl)-2-(2,2,2-trifluoroacetamido)acetyl)-6,6-dimethyl-3-azabicyclo[3.1.0]hexane-2-carboxamide (Compound 5)

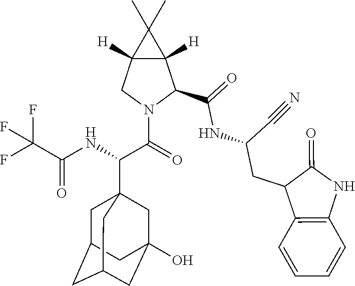 [SMILES: CC1(C)[C@@H]2[C@@H](C(=O)N[C@H](C#N)CC3C(=O)Nc4ccccc43)N(C(=O)[C@@H](NC(=O)C(F)(F)F)C34CC5CC(CC(O)(C5)C3)C4)C[C@@H]21]

The synthetic route for intermediate 5e was as follows:

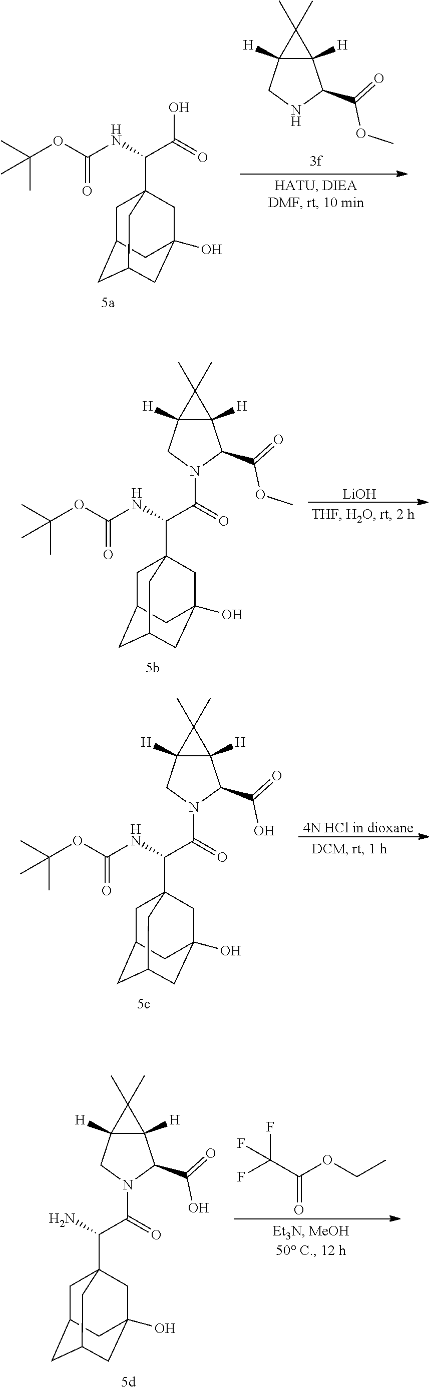

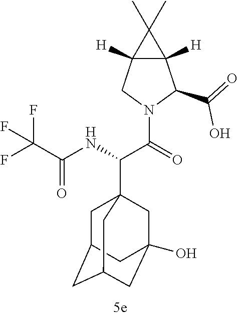 [SMILES: CC1(C)[C@@H]2[C@@H](C(=O)O)N(C(=O)[C@@H](NC(=O)C(F)(F)F)C34CC5CC(CC(O)(C5)C3)C4)C[C@@H]21]

### Step 1: methyl (1R,2S,5S)-3-((S)-2-((tert-butoxycarbonyl)amino)-2-((1r,3R,5R,7S)-3-hydroxyadamantan-1-yl)acetyl)-6,6-dimethyl-3-azabicyclo[3.1.0]hexane-2-carboxylate (5b)

At room temperature, compounds 5a (0.6 g, 1.8 mmol) and 3f (0.28 g, 1.6 mmol) were dissolved in anhydrous N,N-dimethylformamide (5 mL), and O-(7-azabenzotriazol-1-yl)-N,N,N′,N′-tetramethyluronium hexafluorophosphate (0.68 g, 1.8 mmol) was added, followed by the addition of N,N-diisopropylethylamine (0.70 g, 5.4 mmol). The reaction liquid was allowed to react at room temperature for 10 min. Then, the reaction mixture was directly purified by reverse-phase chromatography (SepaFlash® C₁ₛ flash silica gel column, gradient elution with 0.05% aqueous formic acid solution:acetonitrile=20:1-1:20) to obtain compound 5b (0.75 g). m/z (ESI): 477 [M+H]⁺.

### Step 2: (1R,2S,5S)-3-((S)-2-((tert-butoxycarbonyl)amino)-2-((1r,3R,5R,7S)-3-hydroxyadamantan-1-yl)acetyl)-6,6-dimethyl-3-azabicyclo[3.1.0]hexane-2-carboxylic acid (5c)

Compound 5b (0.75 g, 1.6 mmol) was dissolved in tetrahydrofuran (5.0 mL) and water (5.0 mL), and then lithium hydroxide (0.20 g, 4.8 mmol) was added. The reaction liquid was allowed to react at 20° C. for 2 h. Then, the reaction liquid was adjusted to pH=3 by adding 1 M hydrochloric acid and distilled under reduced pressure to remove the solvent. The resulting residue was directly purified by reverse-phase chromatography (SepaFlash® C₁₈ flash silica gel column, gradient elution with 0.05% aqueous formic acid solution:acetonitrile=20:1-1:20) to obtain compound 5c (0.72 g). m/z (ESI): 463 [M+H]⁺.

### Step 3: (1R,2S,5S)-3-((S)-2-amino-2-((1r,3R,5R,7S)-3-hydroxyadamantan-1-yl)acetyl)-6,6-dimethyl-3-azabicyclo[3.1.0]hexane-2-carboxylic acid (5d)

At room temperature, compound 5c (0.72 g, 1.5 mmol) was dissolved in 1,4-dioxane (5.0 mL), and a solution of hydrochloric acid in 1,4-dioxane (4.0 M, 5.0 mL, 20 mmol) was added. The reaction liquid was allowed to react at room temperature for 12 h. Then, the reaction liquid was lyophilized and then directly used in the next step.

### Step 4: (1R,2S,5S)-3-((S)-2-((1R,3R,5R,7S)-3-hydroxyadamantan-1-yl)-2-(2,2,2-trifluoroacetamido) acetyl)-6,6-dimethyl-3-azabicyclo[3.1.0]hexane-2-carboxylic acid (5e)

At room temperature, compound 5d (0.58 g, 1.5 mmol) was dissolved in methanol (10 mL), and then triethylamine (0.76 g, 7.5 mmol) and ethyl trifluoroacetate (1.7 g, 12 mmol) were added. The mixture was then heated to 50° C. and allowed to react for 12 h. Then, the reaction liquid was concentrated. The resulting residue was directly purified by reverse-phase chromatography (SepaFlash® C₁₈ flash silica gel column, gradient elution with 0.05% aqueous formic acid solution:acetonitrile=20:1-1:20) to obtain compound 5e (0.64 g). m/z (ESI): 459 [M+H]⁺.

Compound 5 could be prepared by replacing compound 1d with compound 5e and using a method similar to that in Example 1.

m/z (ESI): 642 [M+H]⁺.

¹1HNMR: (400 MHz, DMSO-d₆) δ 10.5 (s, 1H), 9.36 (d, J=9.2 Hz, 1H), 9.28 (d, J=9.2 Hz, 1H), 7.39 (d, J=8.0 Hz, 1H), 7.18 (t, J=8.0 Hz, 1H), 6.90 (t, J=8.0 Hz, 1H), 6.82 (d, J=8.0 Hz, 1H), 5.28-5.23 (m, 1H), 4.31 (s, 1H), 4.26 (d, J=7.6 Hz, 1H), 4.20 (s, 1H), 3.92-3.89 (m, 1H), 3.69-3.66 (m, 1H), 3.54-3.47 (m, 1H), 2.34-2.27 (m, 1H), 2.14-1.83 (m, 2H), 1.60-1.28 (m, 15H), 1.04 (s, 3H), 0.86 (s, 3H).

### Example 6: Preparation of (1R,2S,5S)—N—((S)-1-cyano-2-((S)-2-oxopyrrolidin-3-yl)ethyl)-3-(2-(4-(dimethylamino)cyclohexane)-2-(2,2,2-trifluoroacetamido)acetyl)-6,6-dimethyl-3-azabicyclo[3.1.0]cyclohexane-2-carboxamide (Compound 6)

The synthetic route was as follows:

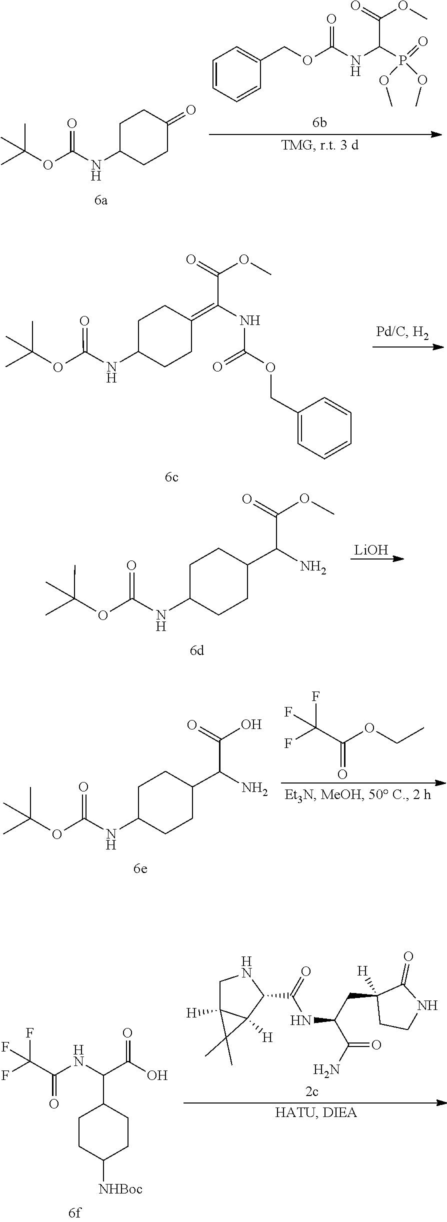

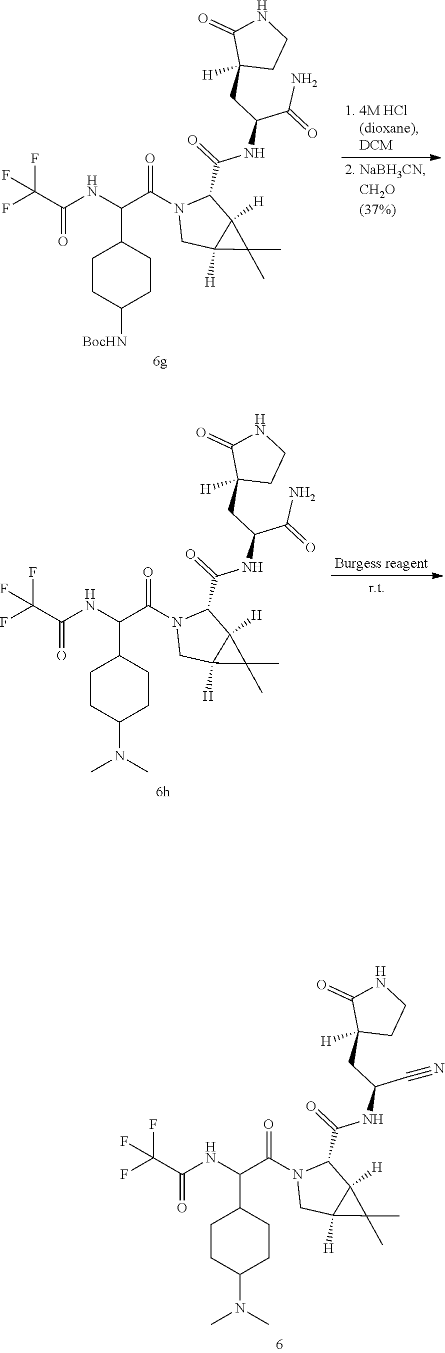 [SMILES: CC(C)(C)OC(=O)NC1CCC(C(NC(=O)C(F)(F)F)C(=O)N2C[C@H]3[C@@H]([C@H]2C(=O)N[C@@H](C[C@@H]2CCNC2=O)C(N)=O)C3(C)C)CC1 ; CN(C)C1CCC(C(NC(=O)C(F)(F)F)C(=O)N2C[C@H]3[C@@H]([C@H]2C(=O)N[C@@H](C[C@@H]2CCNC2=O)C(N)=O)C3(C)C)CC1 ; CN(C)C1CCC(C(NC(=O)C(F)(F)F)C(=O)N2C[C@H]3[C@@H]([C@H]2C(=O)N[C@H](C#N)C[C@@H]2CCNC2=O)C3(C)C)CC1]

### Step 1: methyl 2-((benzyloxy)carbonyl)amino)-2-(4-((tert-butoxycarbonyl)amino) cyclohexylidene acetate (6c)

Benzyloxycarbonyl-α-phosphonoglycine trimethyl ester 6b (1.7 g, 5.3 mmol) was dissolved in ethyl acetate (15 mL), and tetramethylguanidine (0.8 g, 6.8 mmol) was added. The mixture was stirred at room temperature for half an hour, then a solution of 4-N-Boc-aminocyclohexanone 6a (1.7 g, 7.9 mmol) in ethyl acetate (5 mL) was added, and the resulting mixture was allowed to react at room temperature for 3 days. Then, the reaction liquid was concentrated. The resulting residue was directly purified by reverse-phase column chromatography (C18, 0.05% ammonia water:acetonitrile=20:1-1:20) to obtain product 6c (1.1 g, yield: 49%).

m/z (ESI): 419 [M+H]⁺.

### Step 2: methyl 2-amino-2-(4-((tert-butoxycarbonyl)amino)cyclohexyl)acetate (6d)

Compound 6c (0.14 g, 0.3 mmol) was dissolved in ethyl acetate (3 mL) and methanol (3 mL), and 10% palladium on carbon (0.04 g, 0.2 mmol) was added. The mixture was purged with hydrogen 3 times using a hydrogen balloon, then stirred at room temperature under normal pressure for 12 h, and filtered to remove the palladium on carbon. The filtrate was concentrated to dryness by rotary evaporation to obtain a crude product of 6d (90 mg, yield: 93%), which could be directly used in the next step without purification.

m/z (ESI): 287 [M+H]⁺.

### Step 3: 2-amino-2-(4-((tert-butoxycarbonyl)amino)cyclohexyl)acetic acid (6e)

At room temperature, compound 6d (95 mg, 0.33 mmol) and lithium hydroxide (15 mg, 0.66 mmol) were dissolved in a mixed solution of tetrahydrofuran (2 mL)/water (0.5 mL). The mixture was allowed to react at room temperature for 3 h, then concentrated under reduced pressure to remove the THF, and diluted with a small amount of water. The pH was adjusted to neutral with hydrochloric acid (1 M), and the mixture was lyophilized to obtain compound 6e (85 mg, yield: 94%). m/z (ESI): 273 [M+H]⁺.

### Step 4: 2-(4-((tert-butoxycarbonyl)amino)cyclohexyl)-2-(2,2,2-trifluoroacetamido) acetic acid (6f)

Compound 6e (90 mg, 0.33 mmol) was dissolved in methanol (2 mL), and triethylamine (0.20 g, 1.98 mmol) was added. The mixture was stirred for 5 min, then ethyl trifluoroacetate (140 mg, 1.00 mmol) was added, and the resulting mixture was allowed to react at 50° C. for 3 h. The reaction liquid was cooled to room temperature and then distilled under reduced pressure to remove the solvent. The resulting residue was subjected to reverse-phase column chromatography (C18, 0.05% formic acid:acetonitrile=20:1-1:20) to obtain compound 6f (60 mg, yield: 49%).

m/z (ESI): 369 [M+H]⁺.

### Step 5: tert-butyl (4-(2-((1R,2S,5S)-2-(((S)-1-amino-1-oxo-3-((S)-2-oxopyrrolidin-3-yl)propyl-2-yl)carbamoyl)-6,6-dimethyl-3-azabicyclo[3.1.0]hexan-3-yl)-2-oxo-1-(2,2,2-trifluoroacetamido)ethyl)cyclohexyl)-2-carboxamide (6g)

Compound 6f (0.12 g, 0.32 mmol), compound 2c (0.13 g, 0.38 mmol), and O-(7-azabenzotriazol-1-yl)-N,N,N′,N′-tetramethyluronium hexafluorophosphate (0.15 g, 0.38 mmol) were dissolved in anhydrous N,N-dimethylformamide (1.5 mL), and diisopropylethylamine (0.16 g, 1.3 mmol) was added. The mixture was stirred at room temperature for 1 h. Then, the reaction liquid was directly purified by reverse-phase column chromatography (C18, 0.05% ammonia water:acetonitrile=20:1-1:20) to obtain compound 6g (70 mg, yield: 32%).

m/z (ESI): 659.3 [M+H]⁺.

### Step 6: (1R,2S,5S)—N—((S)-1-amino-1-oxo-3-((S)-2-oxopyrrolidin-3-yl)propyl-2-yl)-3-(2-(4-(dimethylamino)cyclohexyl)-2-(2,2,2-trifluoroacetamido)acetyl)-6,6-dimethyl-3-azabicyclo[3.1.0]hexane-2-carboxamide (6h)

Compound 6g (70 mg, 0.11 mmol) was dissolved in dichloromethane (1.0 mL), and a solution of hydrochloric acid in dioxane (1 mL) was added. The mixture was stirred at room temperature for 3 h and then concentrated under reduced pressure to remove the solvent. The resulting residue was redissolved in methanol (2.0 mL), and a 37% aqueous formaldehyde solution (0.1 mL) was added thereto. The mixture was stirred at room temperature for 10 min, and then sodium cyanoborohydride (28 mg, 0.45 mmol) was added. After 1 hour of reaction, the resulting residue was subjected to reverse-phase column chromatography (C18, 0.05% ammonia water:acetonitrile=20:1 to 1:20) to obtain compound 6h (50 mg, yield: 75%).

m/z (ESI): 587 [M+H]⁺.

### Step 7: (1R,2S,5S)—N—((S)-1-cyano-2-((S)-2-oxopyrrolidin-3-yl)ethyl)-3-(2-(4-(dimethylamino) cyclohexane)-2-(2,2,2-trifluoroacetamido)acetyl)-6,6-dimethyl-3-azabicyclo[3.1.0]cyclohexane-2-carboxamide (Compound 6)

At room temperature, 6h (13 mg, 22 μmol) was dissolved in dichloromethane (0.5 mL) and acetonitrile (0.5 mL), and then Burgess reagent (12 mg, 53 μmol) was added. The reaction liquid was stirred for 12 h. The resulting residue was purified by reverse-phase column chromatography (C18, 0.05% ammonia water:acetonitrile=20:1-1:20) to obtain compound 6 (5 mg, yield: 39%).

m/z (ESI): 569 [M+H]⁺.

¹H NMR (400 MHz, DMSO-d₆) δ 9.91 (d, J=7.7 Hz, 1H), 9.04 (d, J=8.5 Hz, 1H), 7.70 (s, 1H), 5.08-4.87 (m, 2H), 4.31 (m, 1H), 4.13 (s, 1H), 3.87 (m, 1H), 3.11-3.04 (m, 4H), 2.27-2.07 (m, 11H), 1.80-1.34 (m, 9H), 1.04-1.01 (m, 4H), 0.84 (s, 3H).

### Example 7: Preparation of (1R,2S,5S)—N—((S)-1-cyano-2-((S)-2-oxopyrrolidin-3-yl)ethyl)-3-(N⁶,N⁶-dimethyl-N²-(2,2,2-trifluoroacetyl)-L-lysyl)-6,6-dimethyl-3-azabicyclo[3.1.0]cyclohexane-2-carboxamide (Compound 7)

The synthetic route was as follows:

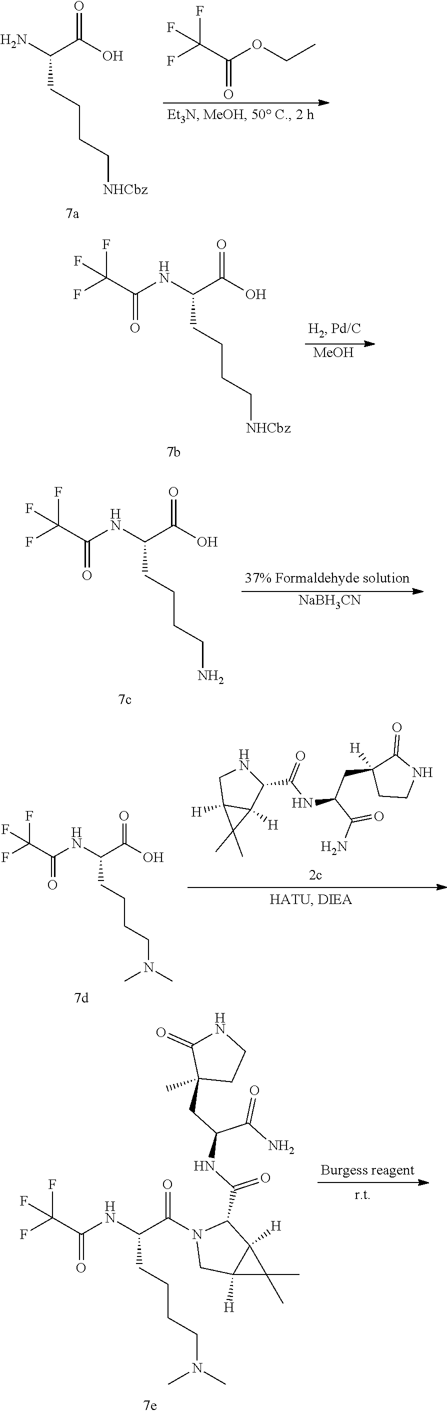

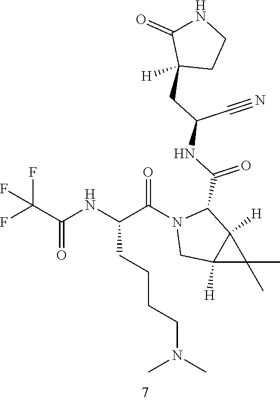 [SMILES: CN(C)CCCC[C@H](NC(=O)C(F)(F)F)C(=O)N1C[C@H]2[C@@H]([C@H]1C(=O)N[C@H](C#N)C[C@@H]1CCNC1=O)C2(C)C]

### Step 1: N⁶-(benzyloxycarbonyl)-N²-(2,2,2-trifluoroacetyl)-L-lysine (7b)

N⁶-Cbz-L-lysine 7a (1.0 g, 3.6 mmol) was dissolved in anhydrous methanol (3.5 mL), and triethylamine (0.7 g, 7.1 mmol) and ethyl trifluoroacetate (0.6 g, 4.6 mmol) were added thereto. The mixture was stirred at room temperature overnight, concentrated under reduced pressure, then diluted with water and adjusted to pH 4, and extracted 3 times with ethyl acetate. The organic phases were combined, washed with saturated brine (100 mL), dried over anhydrous sodium sulfate, and filtered. The filtrate was concentrated to obtain a crude product of 7b (1.0 g, yield: 74%), which was directly used in the next step without purification.

MS (ESI) m/z: 377 [M+H]⁺.

### Step 2: (2,2,2-trifluoroacetyl)-L-lysine (7c)

The crude product of 7b (0.24 g, 0.64 mmol) obtained from the previous step was dissolved in anhydrous methanol (5 mL), and then 10% palladium on carbon (70 mg, 0.63 mmol) was added. The reaction system was purged 3 times using a hydrogen balloon, then stirred at room temperature for 12 h, filtered through celite, and distilled under reduced pressure to remove the solvent to obtain compound 7c (0.14 g, yield: 90%). MS (ESI) m/z: 243 [M+H]⁺.

### Step 3: Synthesis of N⁶,N⁶-dimethyl-N²-(2,2,2-trifluoroacetyl)-L-lysine (7d)

7c (0.14 g, 0.58 mmol) was dissolved in anhydrous methanol (5 mL), and then a 37% aqueous formaldehyde solution (0.5 mL) was added. The mixture was stirred at room temperature for 10 min, and then sodium cyanoborohydride (0.14 g, 2.31 mmol) was added thereto. The resulting mixture was allowed to react at room temperature for 4 h. The resulting residue was purified by reverse-phase column chromatography (C18, 0.05% formic acid:acetonitrile=20:1-1:20) to obtain compound 7d (0.11 g, yield: 70%).

MS (ESI) m/z: 271 [M+H]⁺.

### Step 4: (1R,2S,5S)—N—((S)-1-amino-1-oxo-3-((S)-2-oxopyrrolidin-3-yl)propan-2-yl)-3-(N⁶,N⁶-dimethyl-N²-(2,2,2-trifluoroacetyl)-L-lysyl)-6,6-dimethyl-3-azabicyclo[3.1.0]hexane-2-carboxamide (7e)

Compound 7d (100 mg, 0.26 mmol), compound 2c (89 mg, 0.26 mmol), and O-(7-azabenzotriazol-1-yl)-N,N,N′,N′-tetramethyluronium hexafluorophosphate (117 mg, 0.31 mmol) were dissolved in N,N-dimethylformamide (1.5 mL), and diisopropylethylamine (134 mg, 1.04 mmol) was added. The mixture was stirred at room temperature for 1 h. The resulting residue was subjected to reverse-phase column chromatography (C18, 0.05% formic acid:acetonitrile=20:1-1:20) to obtain compound 7e (56 mg, yield: 38%).

m/z (ESI): 561 [M+H]⁺.

### Step 5: (1R,2S,5S)—N—((S)-1-cyano-2-((S)-2-oxopyrrolidin-3-yl)ethyl)-3-(N⁶,N⁶-dimethyl-N²-(2,2,2-trifluoroacetyl)-L-lysyl)-6,6-dimethyl-3-azabicyclo[3.1.0]cyclohexane-2-carboxamide (7)

At room temperature, 7e (34 mg, 60 μmol) was dissolved in N,N-dimethylacetamide (1 mL), and then Burgess reagent (74 mg, 314 μmol) was added. The mixture was stirred at room temperature for 12 h. The resulting residue was purified by reverse-phase column chromatography (C18, 0.05% formic acid:acetonitrile=20:1-1:20) to obtain compound 7 (15 mg, yield: 45%).

m/z (ESI): 543 [M+H]⁺.

¹H NMR (400 MHz, DMSO-d₆) δ 9.77 (d, J=6.7 Hz, 1H), 8.97 (d, J=8.1 Hz, 1H), 8.20 (s, 1H), 7.73 (s, 1H), 4.95-4.94 (m, 2H), 4.37-4.35 (m, 1H), 4.14 (s, 1H), 3.88-3.80 (m, 1H), 3.74-3.71 (m, 1H), 3.19-3.08 (m, 2H), 2.44-2.28 (m, 3H), 2.26-2.07 (m, 6H), 1.83-1.55 (m, 5H), 1.49-1.19 (m, 5H), 1.03 (s, 3H), 0.90 (s, 3H).

### Example 8: (1R,2S,5S)—N-((1S)-1-Cyano-2-(2-carbonyl-2,3-dihydro-1H-pyrrolo[2,3-b]pyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 8)

The synthetic route was as follows:

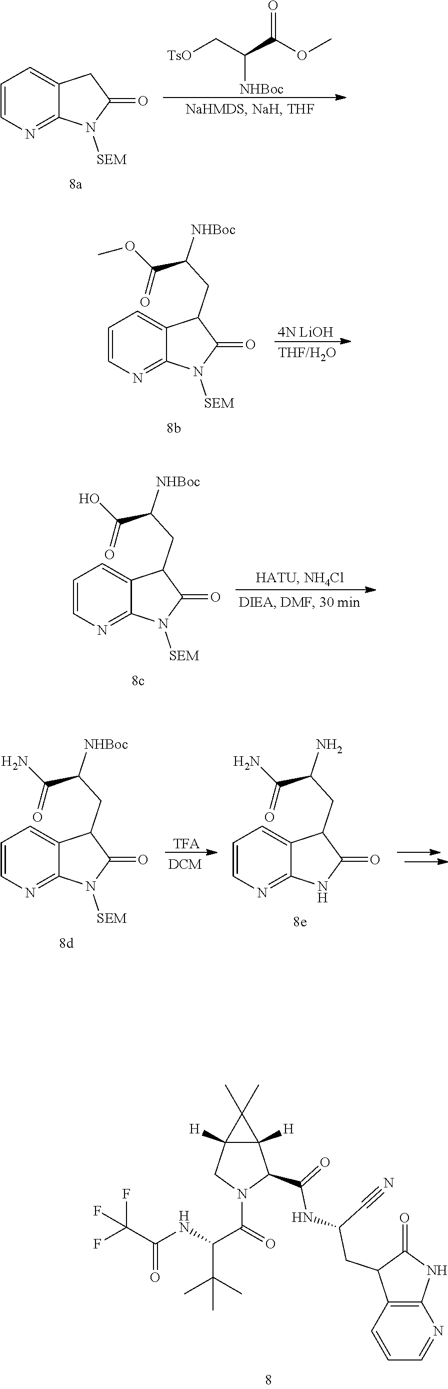

### Step 1: methyl (2S)-2-((tert-butoxycarbonyl)amino)-3-(2-carbonyl-1-((2-(trimethylsilyl)ethoxy)methyl)-2,3-dihydro-1H-pyrrolo[2,3-b]pyridin-3-yl)propanoate (8b)

At room temperature, 1-[[2-(trimethylsilyl)ethoxy]methyl]-1H-pyrrolo[2,3-b]pyridin-2(3H)-one (1.0 g, 3.78 mmol) was dissolved in anhydrous tetrahydrofuran (20 mL), and then the system was purged with nitrogen. A solution of sodium bis(trimethylsilyl)amide in tetrahydrofuran (2.25 mL, 4.5 mmol, 2 M/L) was then added dropwise to the reaction liquid at −78° C., and after the addition, the mixture was allowed to react at this temperature for 30 min. Meanwhile, at −10° C., Boc-Ser(Tos)-OMe (1.4 g, 3.78 mmol) was dissolved in anhydrous tetrahydrofuran, then sodium hydride (0.11 g, 4.54 mmol) was added thereto, and the mixture was allowed to react at this temperature for 30 min. Then, this solution was added dropwise to the above anhydrous tetrahydrofuran solution of 1-[[2-(trimethylsilyl)ethoxy]methyl]-1H-pyrrolo[2,3-b]pyridin-2(3H)-one treated with sodium bis(trimethylsilyl)amide, and the resulting mixture was allowed to react at −78° C. for 2 h, then warmed to room temperature, and allowed to react for another 2 h. The reaction liquid was quenched with a saturated aqueous sodium chloride solution and then concentrated. The resulting residue was directly purified by reverse-phase chromatography (SepaFlash® C₁₈ flash silica gel column, gradient elution with 0.05% aqueous formic acid solution:acetonitrile=20:1-1:20) to obtain compound 8b (1.8 g). m/z (ESI): 466.

### Step 2: (2S)-2-((tert-butoxycarbonyl)amino)-3-(2-carbonyl-1-((2-(trimethylsilyl)ethoxy) methyl)-2,3-dihydro-1H-pyrrolo[2,3-b]pyridin-3-yl)propanoic acid (8c)

At room temperature, compound 8b (0.95 g, 2.0 mmol) was dissolved in an anhydrous tetrahydrofuran solution, and then an aqueous lithium hydroxide solution (2.5 mL, 10 mmol, 4 M/L) was added. The mixture was stirred at room temperature for 30 min. Then, the reaction liquid was adjusted to pH=3 by adding 1 M hydrochloric acid and concentrated. The resulting residue was directly purified by reverse-phase chromatography (SepaFlash® C₁₈ flash silica gel column, gradient elution with 0.05% aqueous formic acid solution:acetonitrile=20:1-1:20) to obtain compound 8c (0.70 g). m/z (ESI): 452.

### Step 3: tert-butyl ((2S)-1-amino-1-oxo-3-(2-oxo-1-((2-(trimethylsilyl)ethoxy)methyl)-2,3-dihydro-1H-pyrrolo[2,3-b]pyridin-3-yl)propan-2-yl)carbamate (8d)

At room temperature, compound 8c (0.7 g, 1.6 mmol) and ammonium chloride (0.16 g, 3.2 mmol) were dissolved in anhydrous N,N-dimethylformamide (5 mL), and then O-(7-azabenzotriazol-1-yl)-N,N,N′,N′-tetramethyluronium hexafluorophosphate (0.88 g, 2.3 mmol) and N,N-diisopropylethylamine (0.6 g, 4.6 mmol) were added. The mixture was allowed to react at room temperature for 1 h. Then, the reaction mixture was directly purified by reverse-phase chromatography (SepaFlash® C₁₈ flash silica gel column, gradient elution with 0.05% aqueous formic acid solution:acetonitrile=20:1-1:20) to obtain compound 8d (0.60 g). m/z (ESI): 451 [M+H]⁺.

### Step 4: (2S)-2-amino-3-(2-oxo-2,3-dihydro-1H-pyrrolo[2,3-b]pyridin-3-yl)acrylamide (8e)

Compound 8d (0.60 g, 1.3 mmol) was dissolved in TFA (5 mL), and the mixture was allowed to react at room temperature for 30 min. Then, the reaction liquid was lyophilized to obtain compound 8e, which could be directly used in the next step without purification.

Compound 8 was prepared by the same preparation method as in Example 1 except for replacing compound 1c with compound 8e.

m/z (ESI): 549 [M+H]⁺.

¹HNMR: (400 MHz, DMSO-d₆) δ 11.1 (s, 1H), 9.40-9.04 (m, 1H), 8.09-8.01 (m, 1H), 7.72-7.70 (m, 1H), 7.01-6.91 (m, 1H), 4.39-4.33 (m, 1H), 4.20 (s, 1H), 3.70-3.28 (m, 1H), 3.05-2.98 (m, 2H), 2.58-2.52 (m, 1H), 2.35-2.31 (m, 1H), 1.66-1.59 (m, 1H), 1.37 (d, J=10.0 Hz, 1H), 1.26-1.20 (m, 2H), 1.04-0.78 (m, 15H).

### Example 9: (1R,2S,5S)-3-((S)-2-(2-Chloro-2,2-difluoroacetamido)-3,3-dimethylbutanoyl)-N-((1S)-1-cyano-2-(2-oxoindol-3-yl)ethyl)-6,6-dimethyl-3-azabicyclo[3.1.0]hexane-2-carboxamide (Compound 9)

The synthetic route was as follows:

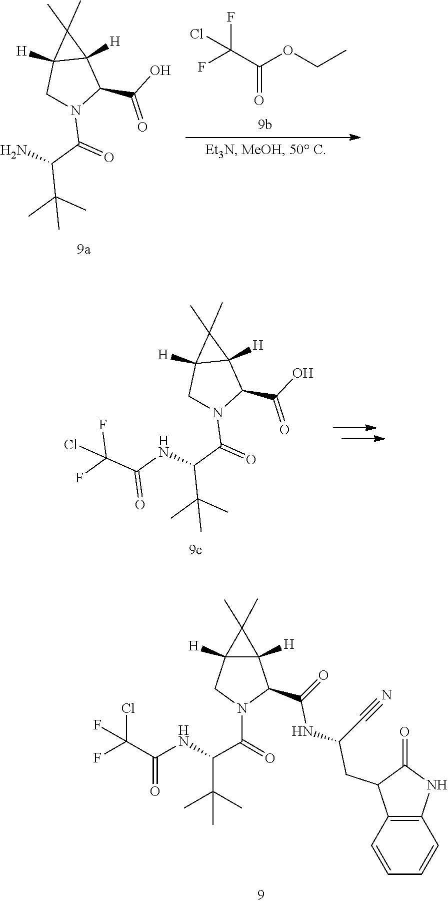

### Step 1: (1R,2S,5S)-3-((S)-2-(2-chloro-2,2-difluoroacetamido)-3,3-dimethylbutanoyl)-6,6-dimethyl-3-azabicyclo[3.1.0]hexane-2-carboxylic acid (9c)

At 20° C., compound 9a (90 mg, 0.3 mmol) was dissolved in methanol (10 mL), and then triethylamine (0.11 g, 1.1 mmol) and ethyl chlorodifluoroacetate 9b (94 mg, 0.6 mmol) were added. The reaction liquid was heated to 50° C. and allowed to react for 16 h. Then, the reaction liquid was concentrated, adjusted to pH 3-4 by adding 1 M hydrochloric acid, and extracted with ethyl acetate (3×10 mL). The organic phase was dried over anhydrous sodium sulfate, filtered, and concentrated to obtain compound 9c (65 mg). m/z (ESI): 338.1 [M+H]⁺.

### Step 2: (1R,2S,5S)-3-((S)-2-(2-chloro-2,2-difluoroacetamido)-3,3-dimethylbutanoyl)-N-((1S)-1-cyano-2-(2-oxoindol-3-yl)ethyl)-6,6-dimethyl-3-azabicyclo[3.1.0]hexane-2-carboxamide (9)

Compound 9 was prepared by the same method as in Example 1 except for replacing compound 1d with compound 9c.

m/z (ESI): 564 [M+H]⁺.

¹HNMR: (400 MHz, DMSO-d₆) δ 10.50 (s, 1H), 9.31 (d, J=8.0 Hz, 1H), 9.18-9.03 (m, 1H), 7.34 (d, J=7.6 Hz, 1H), 7.18 (t, J=7.6 Hz, 1H), 6.89 (t, J=7.6 Hz, 1H), 6.82 (d, J=7.6 Hz, 1H), 5.25-5.20 (m, 1H), 4.40-4.34 (m, 1H), 4.22 (s, 1H), 3.94-3.90 (m, 1H), 3.69 (d, J=10.0 Hz, 1H), 3.56-3.52 (m, 1H), 2.36-2.28 (m, 1H), 2.18-2.10 (m, 1H), 1.61-1.58 (m, 1H), 1.35 (d, J=7.6 Hz, 1H), 1.03-0.94 (m, 6H), 0.83 (s, 9H).

### Example 10: (1R,2S,5S)—N—((S)-1-Cyano-2-((S)-2-oxopyrrolidin-3-yl)ethyl)-3-((S)-2-((1R,3R,5R,7S)-3-hydroxyadamantan-1-yl)-2-(2,2,2-trifluoroacetamido)acetyl)-6,6-dimethyl-3-azabicyclo[3.1.0]hexane-2-carboxamide (Compound 10)

The synthetic route was as follows:

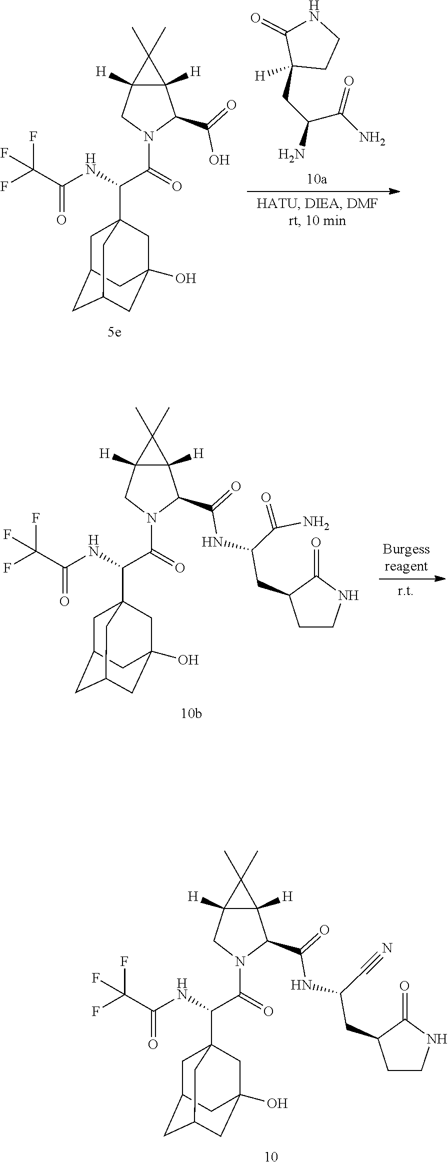

### Step 1: (1R,2S,5S)—N—((S)-1-amino-1-oxo-3-((S)-2-oxopyrrolidin-3-yl)propan-2-yl)-3-((S)-2-((1R,3R,5R,7S)-3-hydroxyadamantan-1-yl)-2-(2,2,2-trifluoroacetamido)acetyl)-6,6-dimethyl-3-azabicyclo[3.1.0]hexane-2-carboxamide (10b)

At room temperature, compounds (1R,2S,5S)-3-((S)-2-((1R,3R,5R,7S)-3-hydroxyadamantan-1-yl)-2-(2,2,2-trifluoroacetamido)acetyl)-6,6-dimethyl-3-azabicyclo[3.1.0]hexane-2-carboxylic acid 5e (92 mg, 0.2 mmol) and (S)-2-amino-3-((S)-2-oxopyrrolidin-3-yl)propanamide 10a (41 mg, 0.24 mmol) were dissolved in anhydrous DMF, then 0-(7-azabenzotriazol-1-yl)-N,N,N′,N′-tetramethyluronium hexafluorophosphate (91 mg, 0.24 mmol) and N,N-diisopropylethylamine (31 mg, 0.24 mmol) were added, and the mixture was allowed to react under this condition for 10 min. Then, the reaction mixture was directly purified by reverse-phase chromatography (SepaFlash® C₁₈ flash silica gel column, gradient elution with 0.05% aqueous formic acid solution:acetonitrile=20:1-1:20) to obtain compound 10b (89 mg, yield: 73%).

m/z (ESI): 612 [M+H]⁺.

### Step 2: (1R,2S,5S)—N—((S)-1-Cyano-2-((S)-2-oxopyrrolidin-3-yl)ethyl)-3-((S)-2-((1R,3R,5R,7S)-3-hydroxyadamantan-1-yl)-2-(2,2,2-trifluoroacetamido)acetyl)-6,6-dimethyl-3-azabicyclo[3.1.0]hexane-2-carboxamide (10)

At room temperature, 10b (89 mg, 0.15 mmol) was dissolved in dichloromethane (0.5 mL), and then Burgess reagent (0.11 g, 0.45 mmol) was added. The mixture was stirred at room temperature for 12 h. Then, the reaction liquid was concentrated. The resulting residue was purified by reverse-phase column chromatography (C18, 0.05% ammonia water:acetonitrile=20:1-1:20) to obtain the target product 10 as a white solid (36 mg, yield: 42%).

m/z (ESI): 594 [M+H]⁺.

¹HNMR: (400 MHz, DMSO-d₆) δ 9.45 (d, J=8.4 Hz, 1H), 9.02 (d, J=8.4 Hz, 1H), 7.68 (s, 1H), 5.08-4.96 (m, 1H), 4.40 (s, 1H), 4.33 (d, J=8.4 Hz, 1H), 4.14 (s, 1H), 3.93-3.89 (m, 1H), 3.67 (d, J=8.8 Hz, 1H), 3.16-3.07 (m, 2H), 2.46-2.33 (m, 1H), 2.16-2.06 (m, 4H), 1.72-1.39 (m, 15H), 1.32 (d, J=7.6 Hz, 1H), 1.03 (s, 3H), 0.85 (s, 3H).

Referring to the synthetic routes in the above examples, the following compounds were synthesized, and their structure and mass spectrometry data are as follows:

| Compound | Structure | m/z [M + H]⁺ |
|---|---|---|
| 11 | 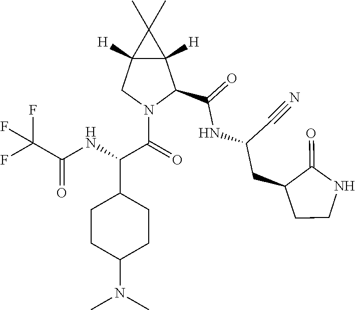 [SMILES: CN(C)C1CCC([C@H](NC(=O)C(F)(F)F)C(=O)N2C[C@H]3[C@@H]([C@H]2C(=O)N[C@H](C#N)C[C@@H]2CCNC2=O)C3(C)C)CC1] | 569 |
| 12 | 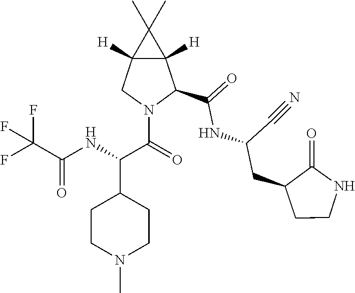 [SMILES: CN1CCC([C@H](NC(=O)C(F)(F)F)C(=O)N2C[C@H]3[C@@H]([C@H]2C(=O)N[C@H](C#N)C[C@@H]2CCNC2=O)C3(C)C)CC1] | 541 |
| 13 | 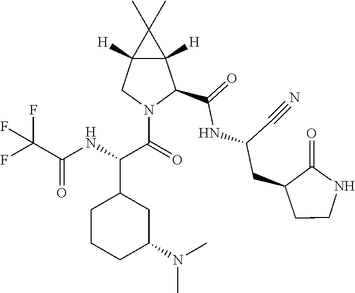 [SMILES: CN(C)[C@@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1] | 569 |
| 14 | 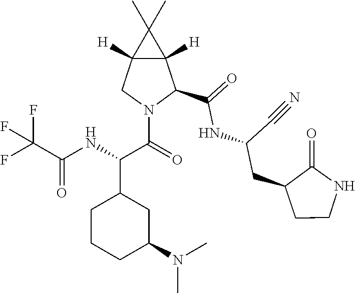 [SMILES: CN(C)[C@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1] | 569 |
| 15 | 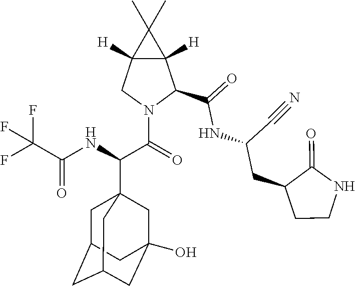 [SMILES: CC1(C)[C@@H]2[C@@H](C(=O)N[C@H](C#N)C[C@@H]3CCNC3=O)N(C(=O)[C@H](NC(=O)C(F)(F)F)C34CC5CC(CC(O)(C5)C3)C4)C[C@@H]21] | 594 |
| 16 | 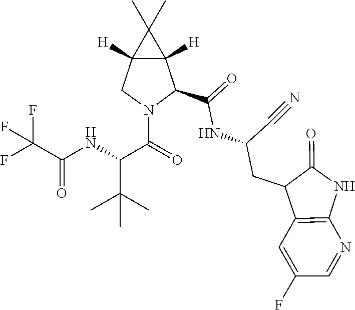 [SMILES: CC(C)(C)[C@H](NC(=O)C(F)(F)F)C(=O)N1C[C@H]2[C@@H]([C@H]1C(=O)N[C@H](C#N)CC1C(=O)Nc3ncc(F)cc31)C2(C)C] | 567 |
| 17 | 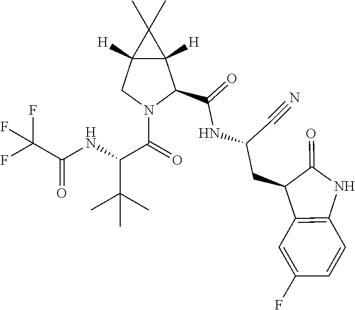 [SMILES: CC(C)(C)[C@H](NC(=O)C(F)(F)F)C(=O)N1C[C@H]2[C@@H]([C@H]1C(=O)N[C@H](C#N)C[C@H]1C(=O)Nc3ccc(F)cc31)C2(C)C] | 566 |
| 18 | 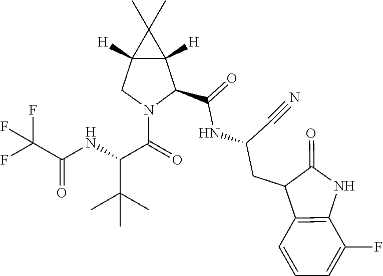 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(F)cccc31)C2(C)C] | 566 |
| 19 | 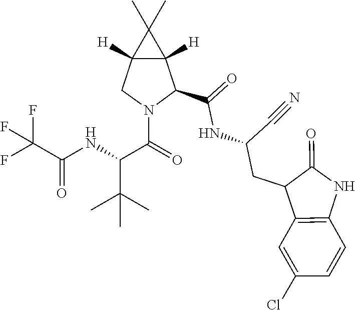 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(Cl)cc31)C2(C)C] | 582 |
| 20 | 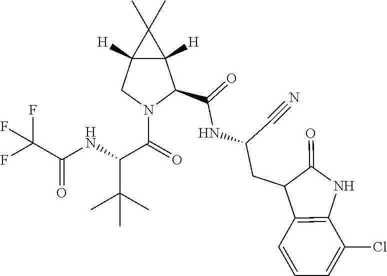 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(Cl)cccc31)C2(C)C] | 582 |
| 21 | 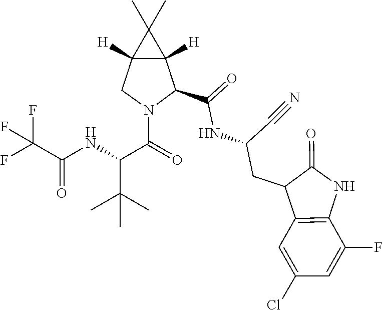 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(F)cc(Cl)cc31)C2(C)C] | 600 |
| 22 | 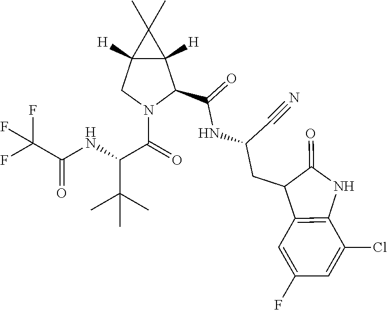 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(F)cc31)C2(C)C] | 600 |
| 23 | 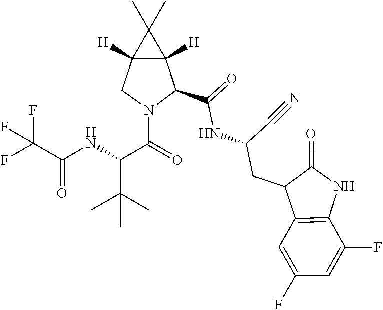 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(F)cc(F)cc31)C2(C)C] | 584 |
| 24 | 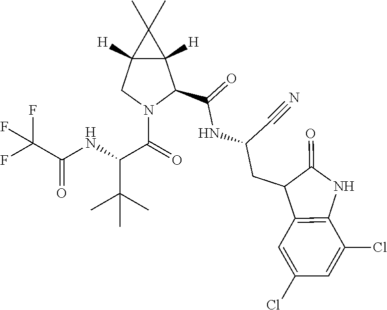 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(Cl)cc31)C2(C)C] | 584 |
| 25 | 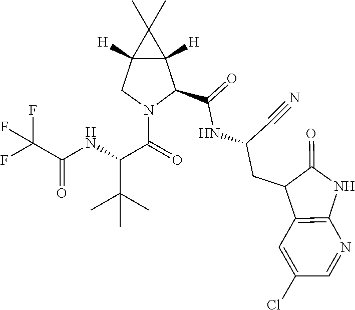 [SMILES: CC(C)(C)[C@H](NC(=O)C(F)(F)F)C(=O)N1C[C@H]2[C@@H]([C@H]1C(=O)N[C@H](C#N)CC1C(=O)Nc3ncc(Cl)cc31)C2(C)C] | 599 |
| 26 | 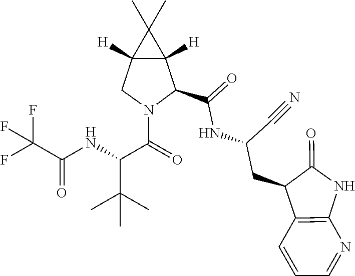 [SMILES: CC(C)(C)[C@H](NC(=O)C(F)(F)F)C(=O)N1C[C@H]2[C@@H]([C@H]1C(=O)N[C@H](C#N)C[C@H]1C(=O)Nc3ncccc31)C2(C)C] | 565 |
| 27 | 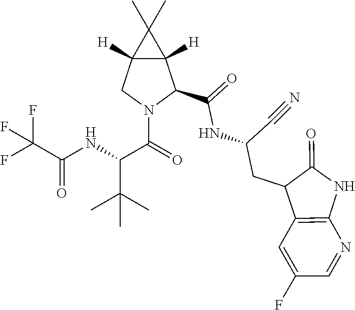 [SMILES: CC(C)(C)[C@H](NC(=O)C(F)(F)F)C(=O)N1C[C@H]2[C@@H]([C@H]1C(=O)N[C@H](C#N)CC1C(=O)Nc3ncc(F)cc31)C2(C)C] | 583 |

### Test Example for Biological Activity and Related Properties

### Test Example 1: Test of Inhibitory Activity Against SARS-COV-2 3CL Protease

### 1. Experimental Instruments and Materials

| Instrument | Manufacturer | Model |
|---|---|---|
| Nano-liter scale acoustic liquid | Labcyte | Echo650 |
| handling system | | |
| Microplate reader | Perkin Elmer | Envision2014 |

The test was performed using the BPS kit (3CL Protease, MBP-tagged (SARS-COV-2) Assay Kit) containing 3CL Protease, a fluorescent substrate, and a reaction buffer. The 3CL protease hydrolyzed the fluorescent substrate to produce a detectable fluorescent product. The protease activity was assessed by detection of the fluorescent product.

The information on the additional reagent and consumable required for the experiment is as follows:

| Reagent | Brand | Catalog No. |
|---|---|---|
| 3CL Protease, MBP-tagged (SARS- | BPS | 79955-2 |
| CoV-2) Assay Kit | | |
| 384-well reaction plate | Perkin Elmer | 6007270 |

### 2. Experimental Procedures

200 nL of 3-fold serially diluted compound (final concentration: 10 μM-0.5 nM) was incubated with 10 μL of 200 nM recombinant 3CL protease (BPS Kit) at room temperature for 30 min. To initiate the reaction, 10 μL of 100 μM fluorescent substrate was added to the reaction plate. Fluorescence signal readings (Ex360/Em460) were taken every 1 minute using Envision (Perkin Elmer) in a continuous reading mode. Fluorescence values were collected over 60 min to calculate the Slope. The inhibition rate of the compound was calculated using the substrate wells as the 100% inhibition control and the enzyme reaction wells as the 0% inhibition control. IC₅₀ was calculated using IDBS XLfit.

### 3. Data Analysis

The inhibition rate of the compound on 3CL protease activity was calculated using the following formula:

$$Inhibition rate \% = ( average value for 0 \% inhibition wells - value for sample well ) / ( average value for 0 \% inhibition wells - average value for 100 \% inhibition wells ) \times 100 \% .$$

Data analysis was performed by XLfit. The concentration-response curves were obtained using non-linear four-parameter fitting, and the IC₅₀ for the compound was calculated:

$$Y = Bottom + ( T op - Bottom ) / ( 1 + 10 \hat{} ( ( Log {IC}_{50} - X ) \times HillSlope ) )$$

where:
X is the Log value of the concentration of the compound;
Y is the percent inhibition rate (% inhibition);
Bottom is the minimum percent inhibition;
Top is the maximum percent inhibition; and
HillSlope is the slope factor of the curve.

The inhibitory effects of the compounds of the present disclosure on 3CL protease activity were determined by the above assay, and the IC₅₀ values obtained are shown in Table 1.

TABLE 1

| Compound No. | IC₅₀ (nM) | IC₅₀ (nM) |
|---|---|---|
| 1-P2 | A | 35.3 |
| 2 | A | 74.1 |
| 3 | B | 525 |
| 4 | B | 273.37 |
| 5 | A | 69.82 |
| 6 | B | 449.44 |
| 7 | C | 1110.29 |
| 8 | A | 33.4 |
| 9 | A | 25.24 |
| 10 | A | 69.8 |

A: <100 nM; B: ≥100 nM and <1000 nM; C: ≥1000 nM and <10000 nM.

1. A compound of formula (I) or a pharmaceutically acceptable salt thereof: 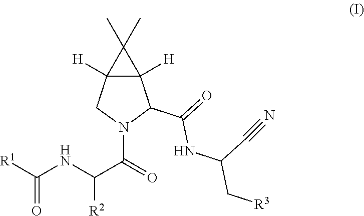
wherein
R¹ is selected from C₁-C₁₀ alkyl, wherein the C₁-C₁₀ alkyl is optionally substituted with halogen;
R² is selected from C₁-C₁₀ alkyl and (CH₂)ₙ—R⁴, wherein n is selected from 0 and 1, and R⁴ is selected from C₃-C₁₂ cycloalkyl, C₄-C₁₂ cycloalkenyl, 4- to 14-membered heterocyclyl, C₆-C₁₀ aryl, and 5- to 10-membered heteroaryl, wherein the C₁-C₁₀ alkyl, C₃-C₁₂ cycloalkyl, C₄-C₁₂ cycloalkenyl, 4- to 14-membered heterocyclyl, C₆-C₁₀ aryl, or 5- to 10-membered heteroaryl is optionally substituted with R^{2a};
R³ is selected from 4- to 14-membered heterocyclyl and 5- to 10-membered heteroaryl, wherein the 4- to 14-membered heterocyclyl or 5- to 10-membered heteroaryl is optionally substituted with R^{3a};
R^{2a} and R^{3a} are each independently selected from F, Cl, Br, I, CN, ═O, OH, NH₂, C₁-C₆ alkyl, C₃-C₆ cycloalkyl, and 4- to 7-membered heterocyclyl, wherein the OH, NH₂, C₁-C₆ alkyl, C₃-C₆ cycloalkyl, or 4- to 7-membered heterocyclyl is optionally substituted with R^{b};
each R^{b} is independently selected from F, Cl, Br, I, CN, ═O, OH, NH₂, C₁-C₆ alkyl, C₃-C₆ cycloalkyl, and 4- to 7-membered heterocyclyl, wherein the OH, NH₂, C₁-C₆ alkyl, C₃-C₆ cycloalkyl, or 4- to 7-membered heterocyclyl is optionally substituted with R^{c};
each R^{c} is independently selected from F, Cl, Br, I, CN, ═O, OH, NH₂, C₁-C₆ alkyl, C₃-C₆ cycloalkyl, 4- to 7-membered heterocyclyl, C₁-C₆ alkoxy, C₃-C₆ cycloalkyloxy, and 4- to 7-membered heterocyclyloxy;
the proviso is that the compound of formula (I) does not comprise

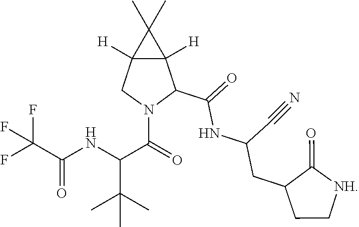 [SMILES: CC(C)(C)C(NC(=O)C(F)(F)F)C(=O)N1CC2C(C1C(=O)NC(C#N)CC1CCNC1=O)C2(C)C]

2. The compound of formula (I) or the pharmaceutically acceptable salt thereof according to claim 1, wherein R¹ is selected from C₁-C₃ alkyl, wherein the C₁-C₃ alkyl is optionally substituted with halogen.

3. The compound of formula (I) or the pharmaceutically acceptable salt thereof according to claim 1, wherein R² is selected from C₁-C₆ alkyl and (CH₂)ₙ—R⁴, wherein n is selected from 0 and 1, and R⁴ is selected from C₅-C₁₂ cycloalkyl, 4- to 7-membered heterocyclyl, and C₆-C₁₀ aryl, wherein the C₁-C₆ alkyl, C₅-C₁₂ cycloalkyl, 4- to 7-membered heterocyclyl, or C₆-C₁₀ aryl is optionally substituted with R^{2a}; or
R² is selected from C₁-C₄ alkyl, C₆-C₁₀ cycloalkyl, 5- to 6-membered heterocyclyl, and CH₂—(C₆-C₁₀ aryl), wherein the C₁-C₄ alkyl, C₆-C₁₀ cycloalkyl, 5- to 6-membered heterocyclyl, or CH₂—(C₆-C₁₀ aryl) is optionally substituted with R^{2a}; or
R² is selected from C₁-C₄ alkyl, C₆-C₁₀ cycloalkyl, and CH₂—(C₆-C₁₀ aryl), wherein the C₁-C₄ alkyl, C₆-C₁₀ cycloalkyl, or CH₂—(C₆-C₁₀ aryl) is optionally substituted with R^{2a}.

4. The compound of formula (I) or the pharmaceutically acceptable salt thereof according to claim 1, wherein R^{2a} is selected from F, Cl, Br, I, CN, ═O, OH, NH₂, and C₁-C₆ alkyl, wherein the OH, NH₂, or C₁-C₆ alkyl is optionally substituted with R^{b}; or
R^{2a} is selected from OH, C₁-C₃ alkyl, and NH₂, wherein the NH₂ is optionally substituted with R^{b}, and R^{b} is selected from C₁-C₃ alkyl.

5. The compound of formula (I) or the pharmaceutically acceptable salt thereof according to claim 1, wherein R² is selected from

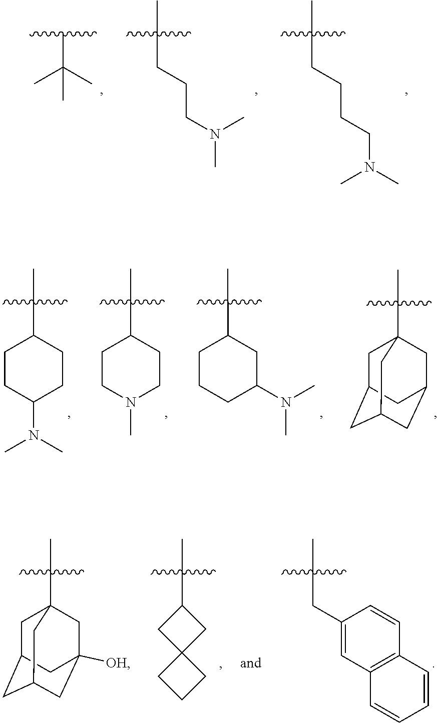

6. The compound of formula (I) or the pharmaceutically acceptable salt thereof according to claim 1, wherein R³ is selected from 4- to 7-membered monocyclic heterocyclyl, 6- to 14-membered fused heterocyclyl, 6- to 14-membered spiro heterocyclyl, and 6- to 14-membered bridged heterocyclyl, wherein the 4- to 7-membered monocyclic heterocyclyl, 6- to 14-membered fused heterocyclyl, 6- to 14-membered spiro heterocyclyl, or 6- to 14-membered bridged heterocyclyl is optionally substituted with R^{3a}; or
R³ is selected from 4- to 7-membered monocyclic heterocyclyl and 6- to 14-membered fused heterocyclyl, wherein the 4- to 7-membered monocyclic heterocyclyl or 6- to 14-membered fused heterocyclyl is optionally substituted with R^{3a}.

7. The compound of formula (I) or the pharmaceutically acceptable salt thereof according to claim 1, wherein R³ is selected from 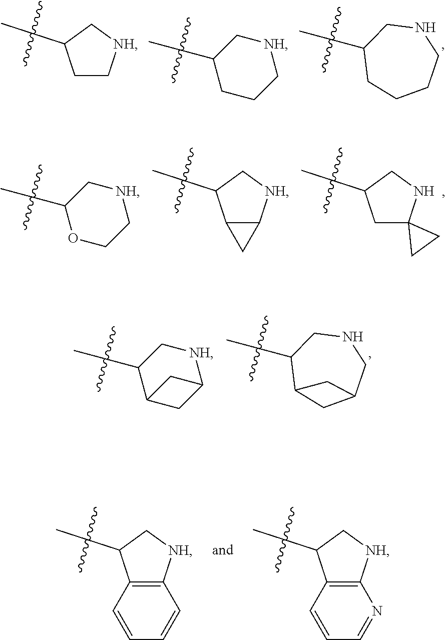
wherein the 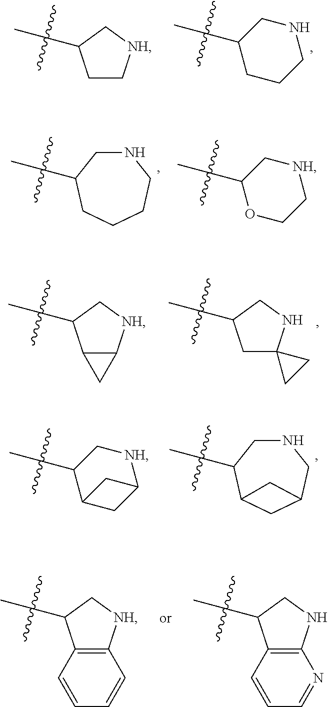
is optionally substituted with R^{3a}; or
R³ is selected from 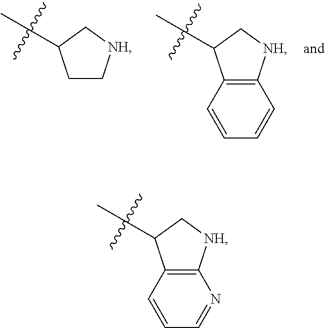 [SMILES: *C1CCNC1 ; *C1CNc2ccccc21 ; *C1CNc2ncccc21]
wherein the 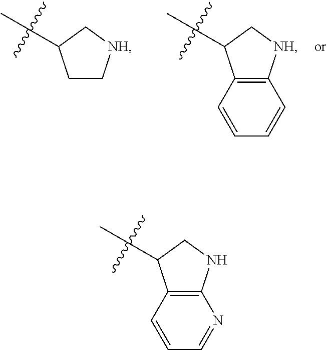 [SMILES: *C1CCNC1 ; *C1CNc2ccccc21 ; *C1CNc2ncccc21]
is optionally substituted with R^{3a}.

8. The compound of formula (I) or the pharmaceutically acceptable salt thereof according to claim 1, wherein R^{3a} is independently selected from F, Cl, Br, I, CN, ═O, OH, NH₂, C₁-C₆ alkyl, C₃-C₆ cycloalkyl, and 4- to 7-membered heterocyclyl; or
R^{3a} is independently selected from F, Cl, Br, I, CN, ═O, and C₁-C₆ alkyl; or
R^{3a} is independently selected from F, Cl, and ═O.

9. The compound of formula (I) or the pharmaceutically acceptable salt thereof according to claim 1, wherein R³ is selected from 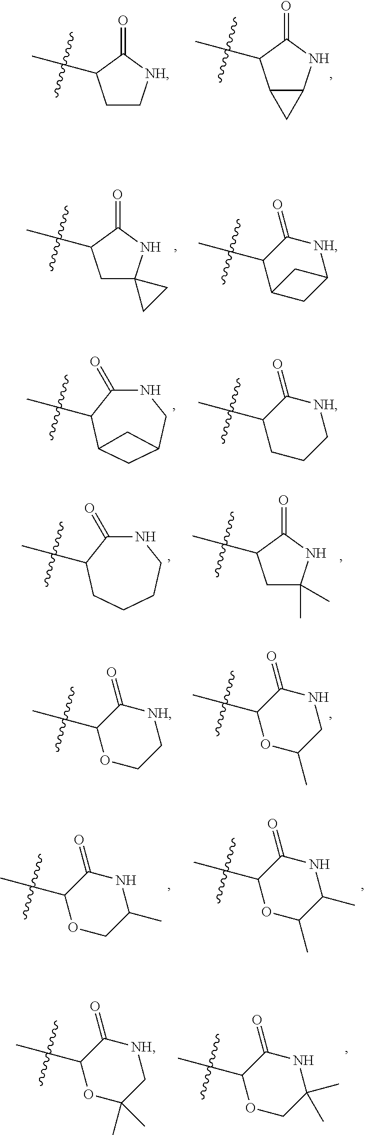 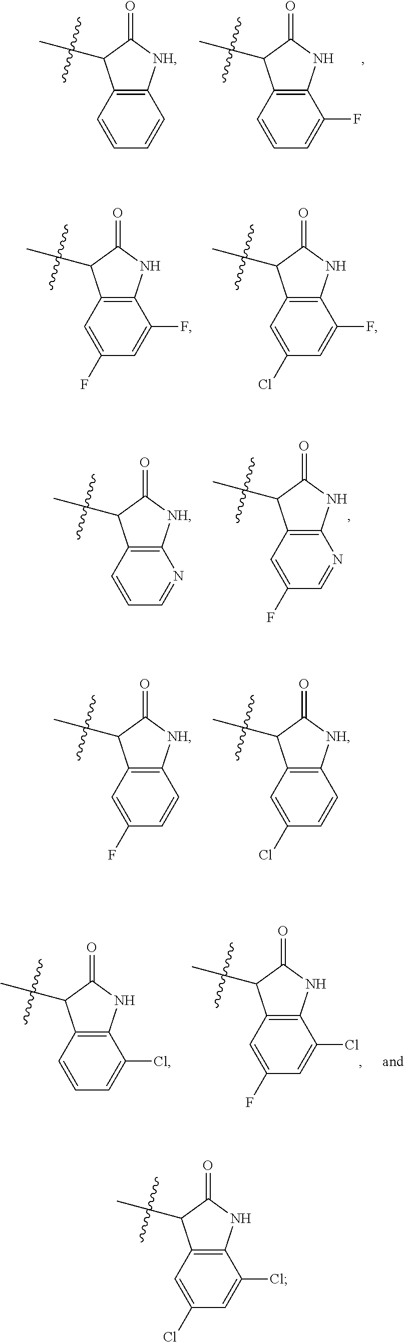
R³ is selected from

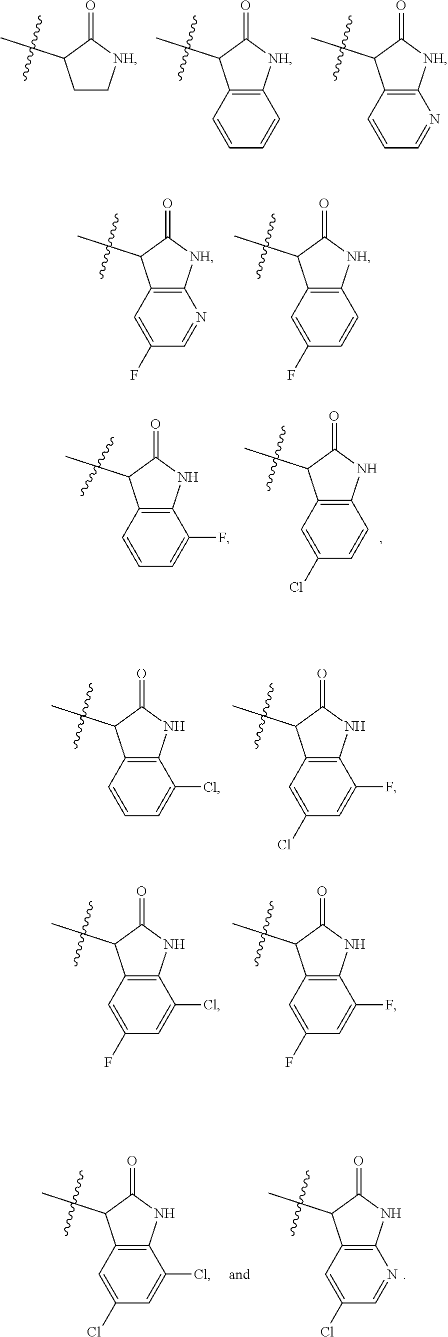

10. The compound of formula (I) or the pharmaceutically acceptable salt thereof according to claim 1, wherein the compound of formula (I) or the pharmaceutically acceptable salt thereof is selected from a compound of formula (Ia) or a pharmaceutically acceptable salt thereof:

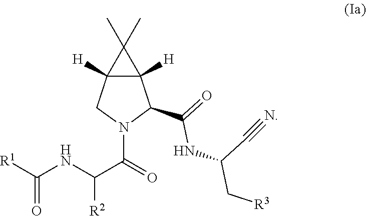

11. The compound of formula (I) or the pharmaceutically acceptable salt thereof according to claim 1, wherein the compound of formula (I) or the pharmaceutically acceptable salt thereof is selected from the following compounds or pharmaceutically acceptable salts thereof:

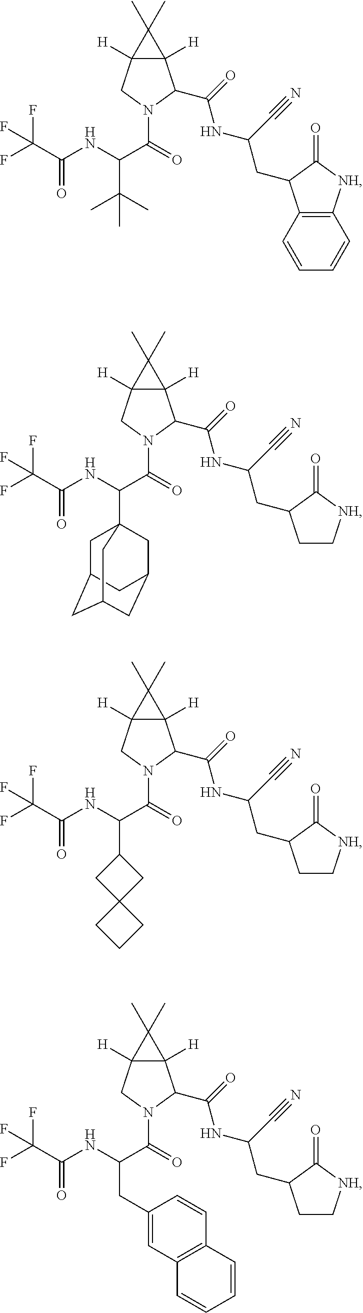

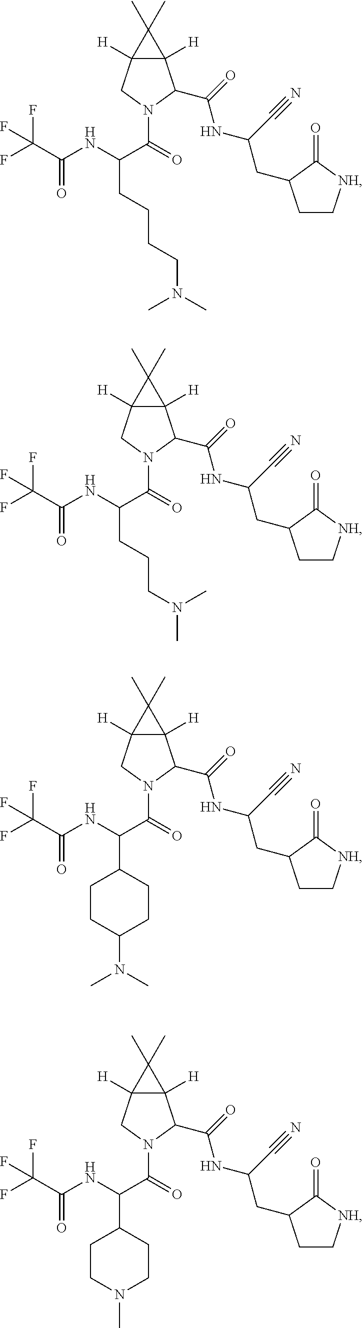

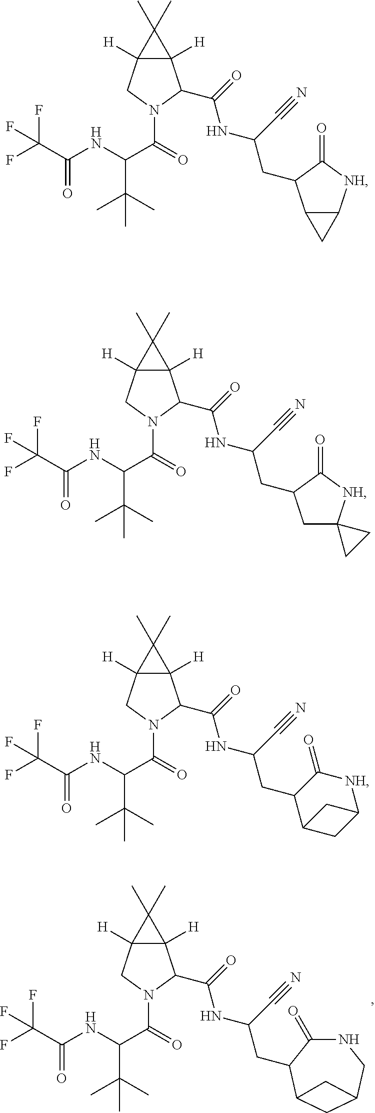

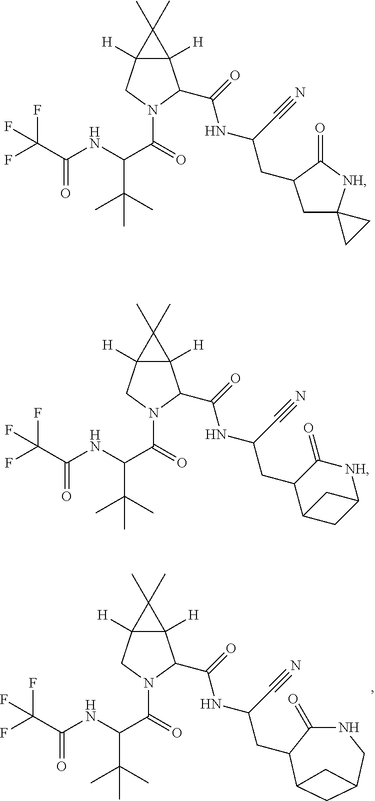

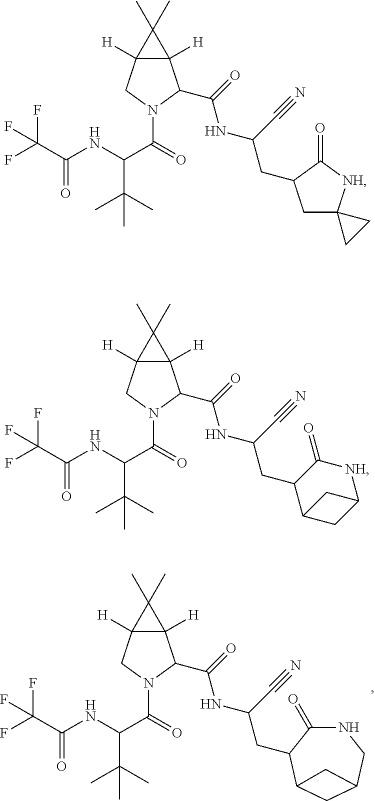

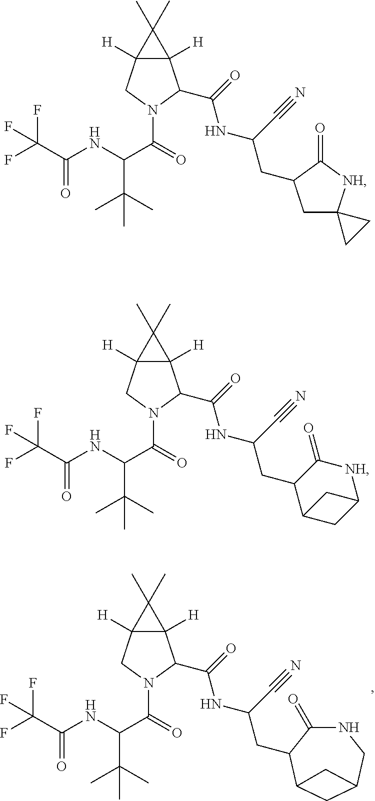

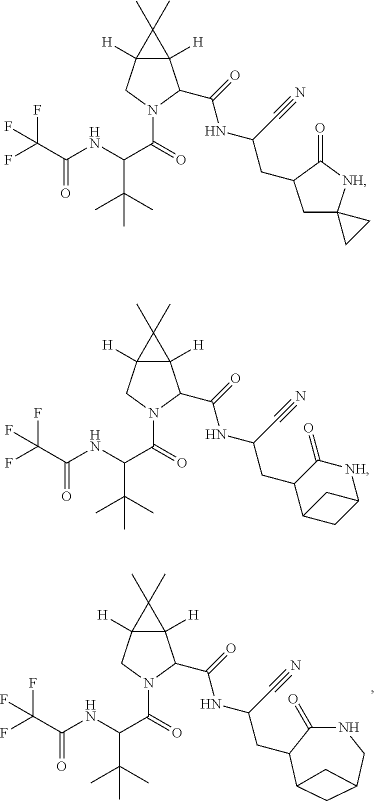

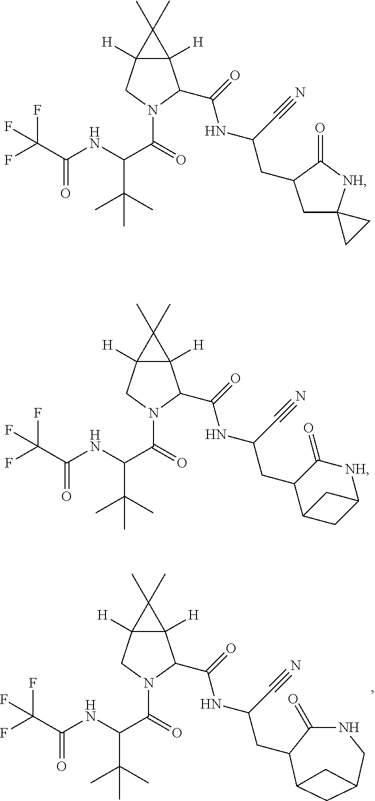

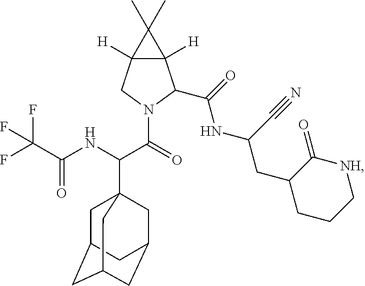 [SMILES: CC1(C)C2CN(C(=O)C(NC(=O)C(F)(F)F)C34CC5CC(CC(C5)C3)C4)C(C(=O)NC(C#N)CC3CCCNC3=O)C21]

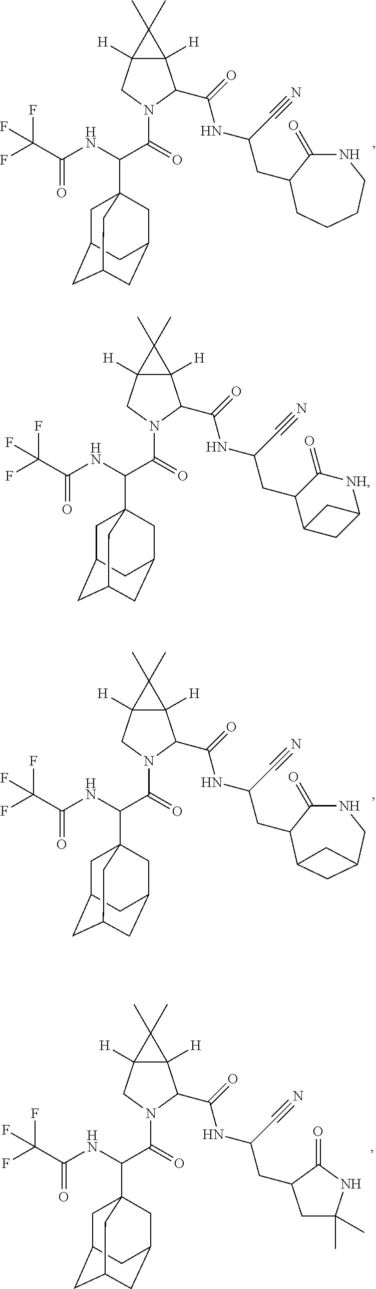

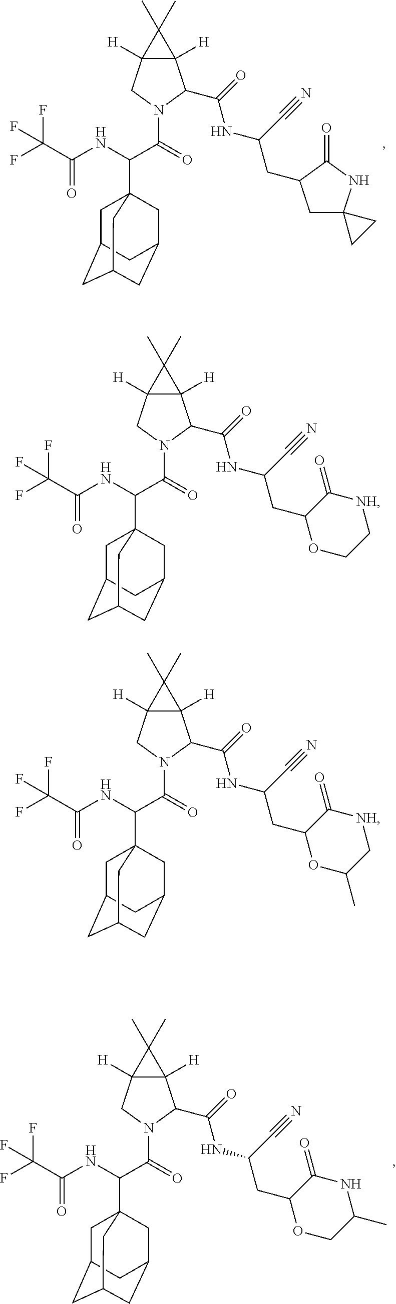

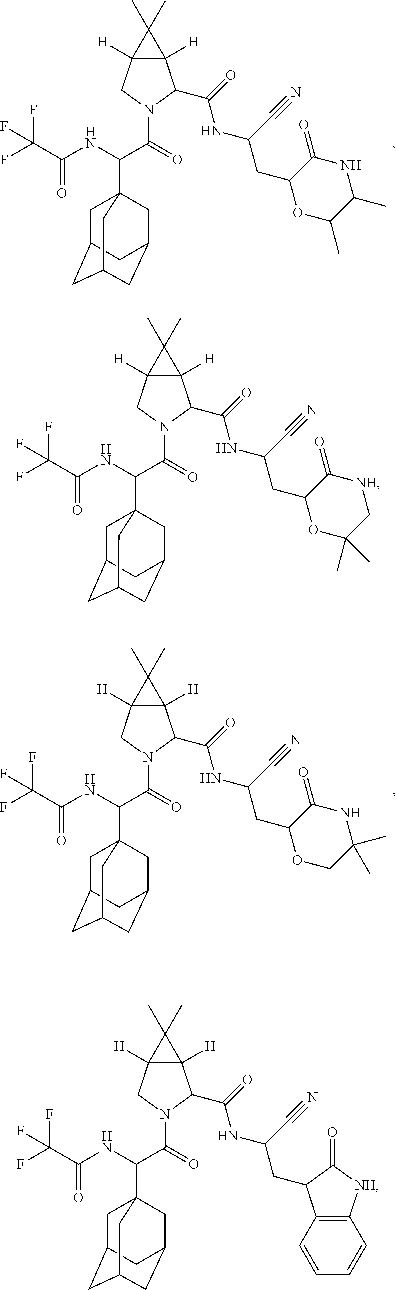

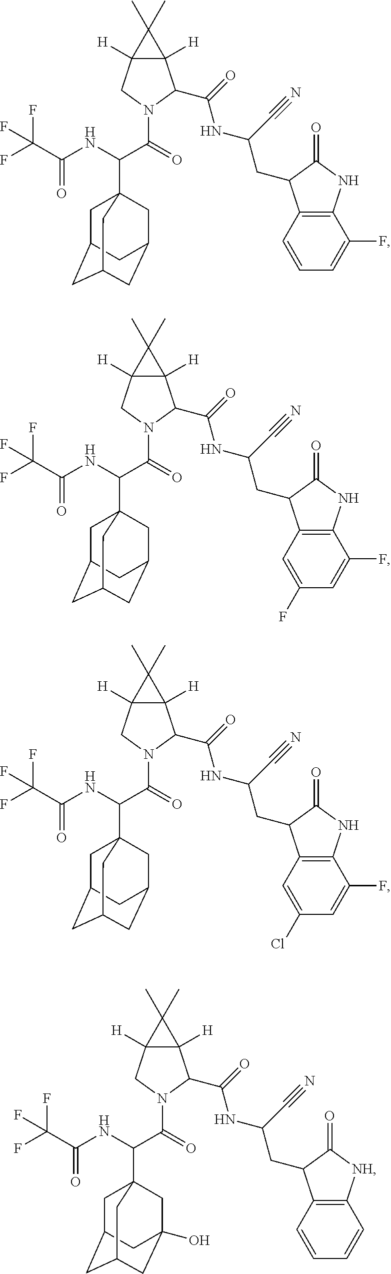

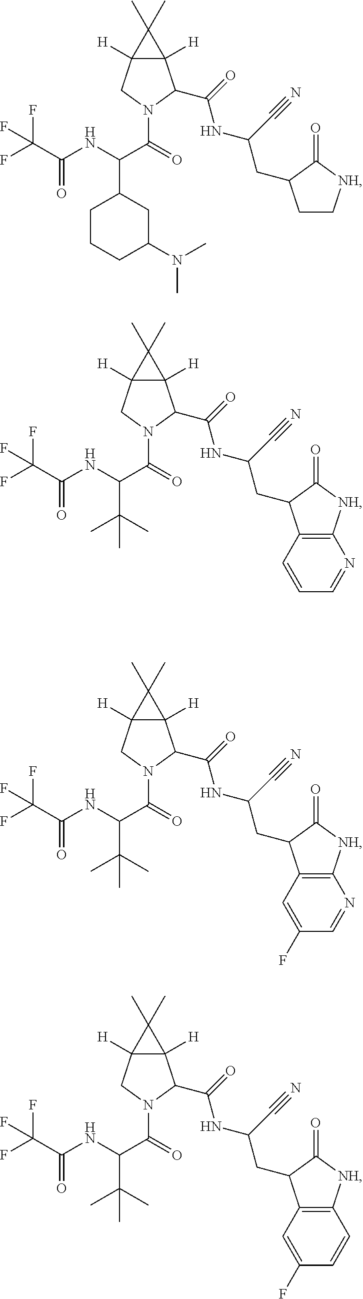

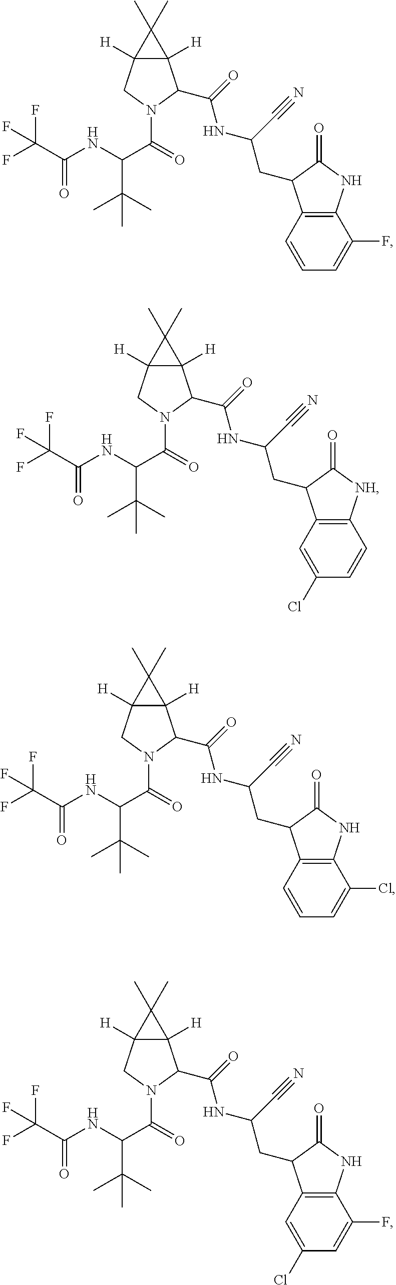 [SMILES: CC(C)(C)C(NC(=O)C(F)(F)F)C(=O)N1CC2C(C1C(=O)NC(C#N)CC1C(=O)Nc3c(F)cccc31)C2(C)C ; CC(C)(C)C(NC(=O)C(F)(F)F)C(=O)N1CC2C(C1C(=O)NC(C#N)CC1C(=O)Nc3ccc(Cl)cc31)C2(C)C ; CC(C)(C)C(NC(=O)C(F)(F)F)C(=O)N1CC2C(C1C(=O)NC(C#N)CC1C(=O)Nc3c(Cl)cccc31)C2(C)C ; CC(C)(C)C(NC(=O)C(F)(F)F)C(=O)N1CC2C(C1C(=O)NC(C#N)CC1C(=O)Nc3c(F)cc(Cl)cc31)C2(C)C]

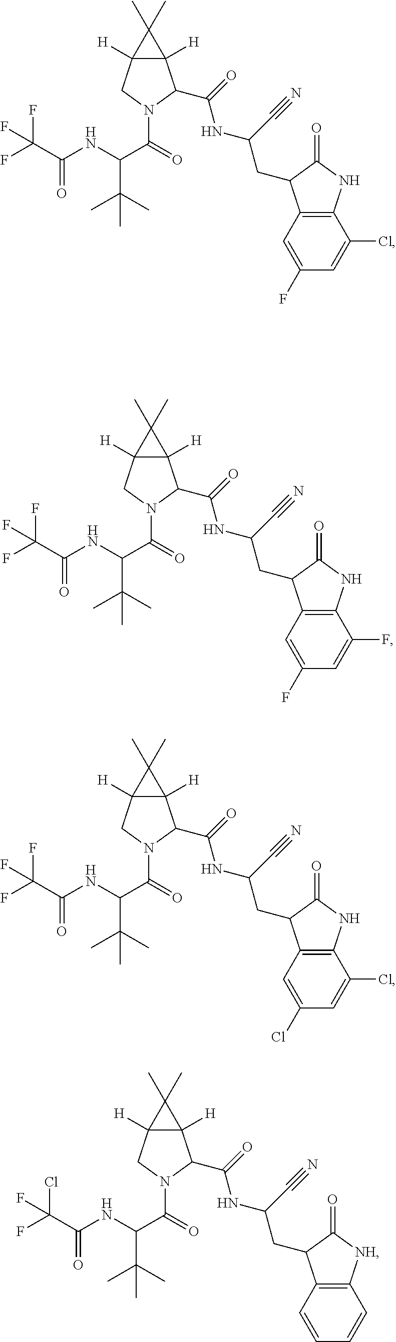 [SMILES: CC(C)(C)C(NC(=O)C(F)(F)F)C(=O)N1CC2C(C1C(=O)NC(C#N)CC1C(=O)Nc3c(Cl)cc(F)cc31)C2(C)C ; CC(C)(C)C(NC(=O)C(F)(F)F)C(=O)N1CC2C(C1C(=O)NC(C#N)CC1C(=O)Nc3c(F)cc(F)cc31)C2(C)C ; CC(C)(C)C(NC(=O)C(F)(F)F)C(=O)N1CC2C(C1C(=O)NC(C#N)CC1C(=O)Nc3c(Cl)cc(Cl)cc31)C2(C)C ; CC(C)(C)C(NC(=O)C(F)(F)Cl)C(=O)N1CC2C(C1C(=O)NC(C#N)CC1C(=O)Nc3ccccc31)C2(C)C]

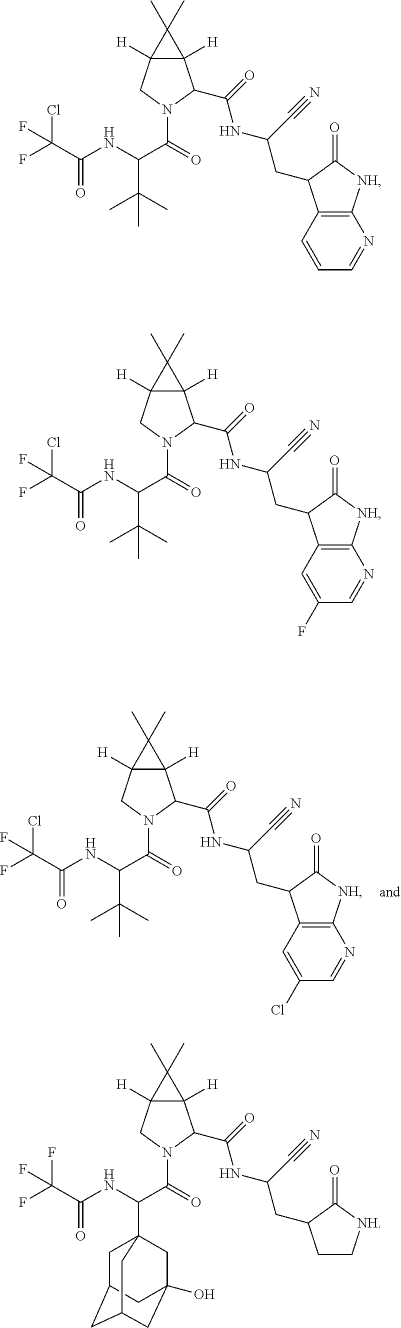 [SMILES: CC(C)(C)C(NC(=O)C(F)(F)Cl)C(=O)N1CC2C(C1C(=O)NC(C#N)CC1C(=O)Nc3ncccc31)C2(C)C ; CC(C)(C)C(NC(=O)C(F)(F)Cl)C(=O)N1CC2C(C1C(=O)NC(C#N)CC1C(=O)Nc3ncc(F)cc31)C2(C)C ; CC(C)(C)C(NC(=O)C(F)(F)Cl)C(=O)N1CC2C(C1C(=O)NC(C#N)CC1C(=O)Nc3ncc(Cl)cc31)C2(C)C ; CC1(C)C2CN(C(=O)C(NC(=O)C(F)(F)F)C34CC5CC(CC(O)(C5)C3)C4)C(C(=O)NC(C#N)CC3CCNC3=O)C21]

12. The compound of formula (I) or the pharmaceutically acceptable salt thereof according to claim 1, wherein the compound of formula (I) or the pharmaceutically acceptable salt thereof is selected from the following compounds or pharmaceutically acceptable salts thereof:

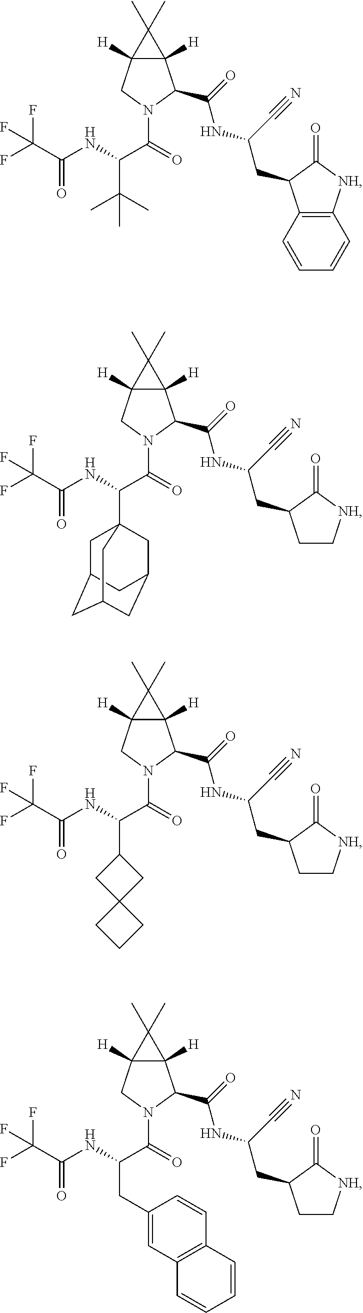

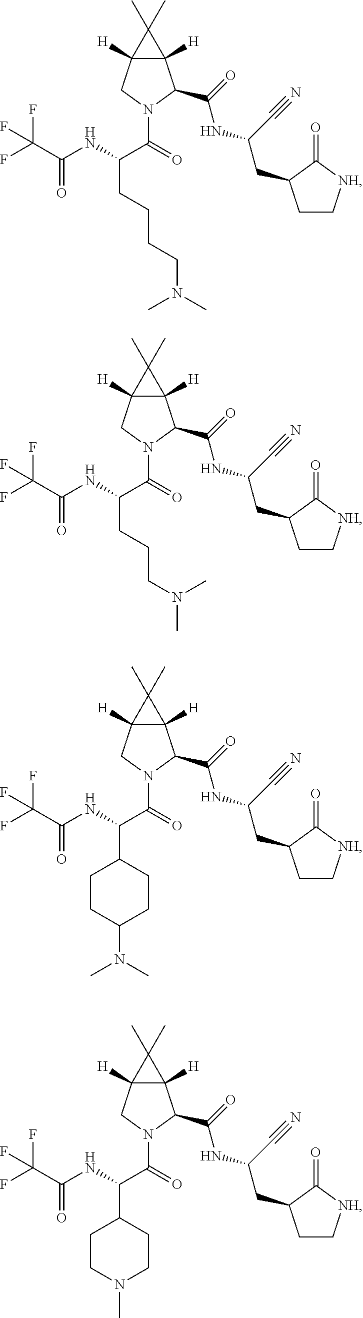

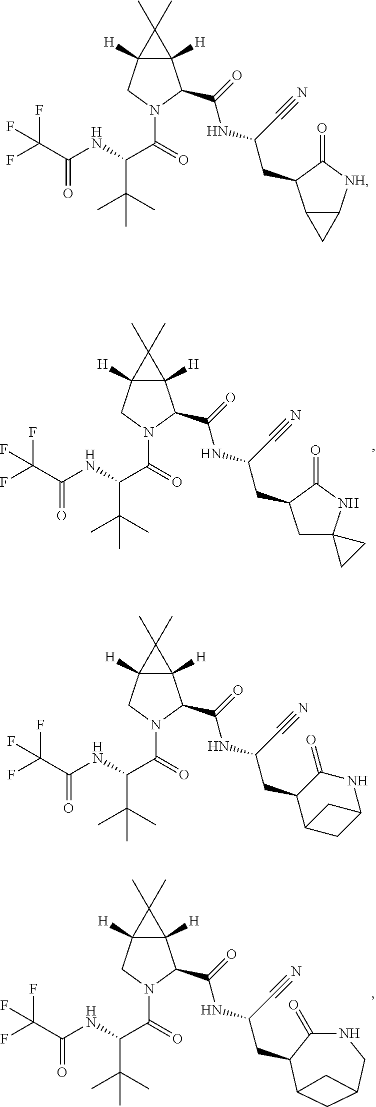

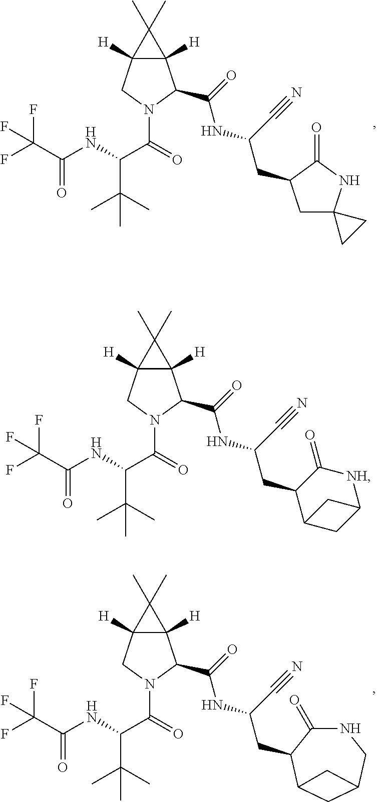

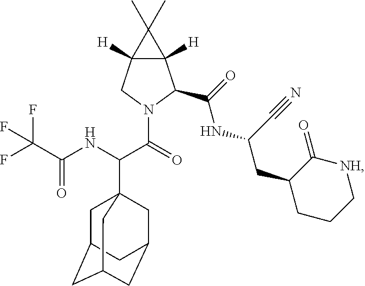 [SMILES: CC1(C)[C@@H]2[C@@H](C(=O)N[C@H](C#N)C[C@@H]3CCCNC3=O)N(C(=O)C(NC(=O)C(F)(F)F)C34CC5CC(CC(C5)C3)C4)C[C@@H]21]

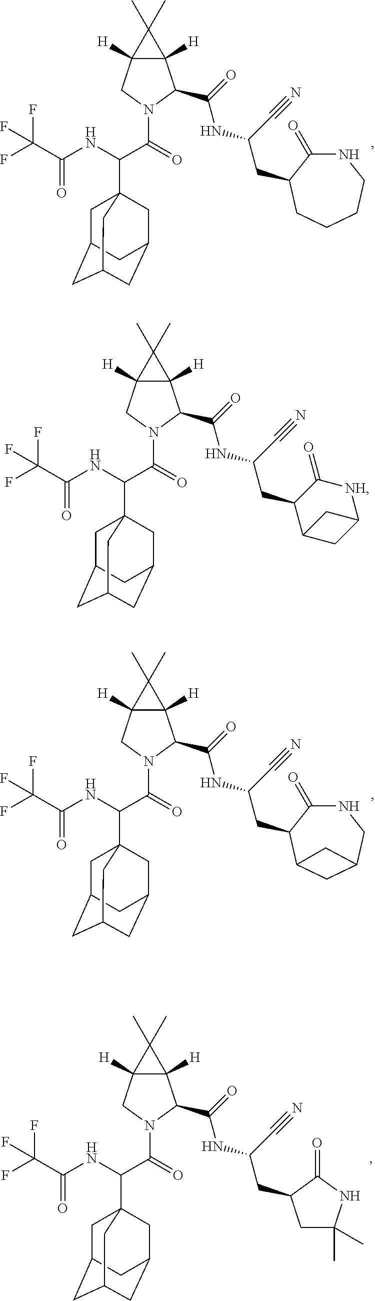

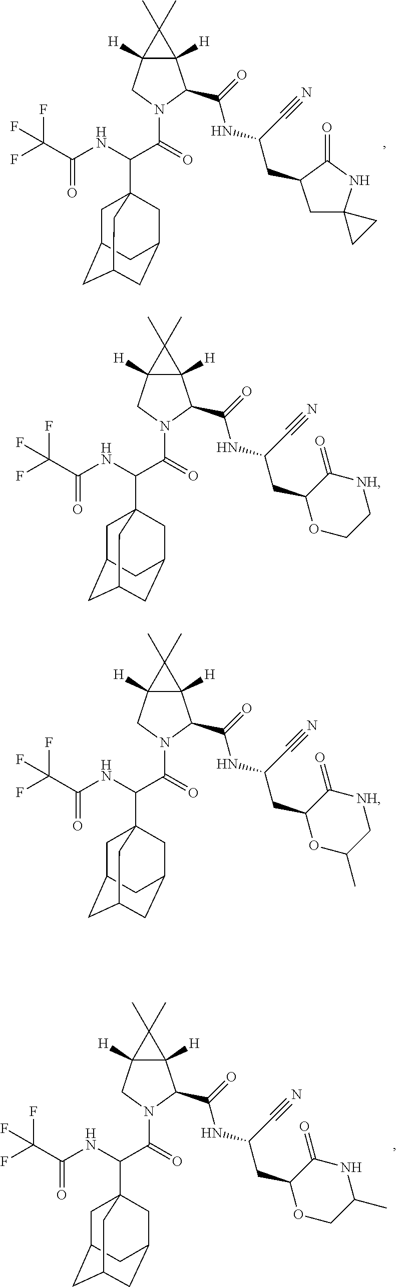

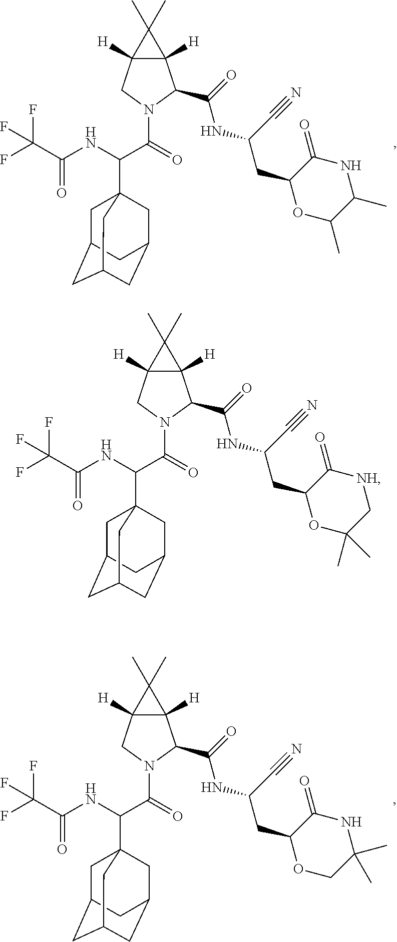

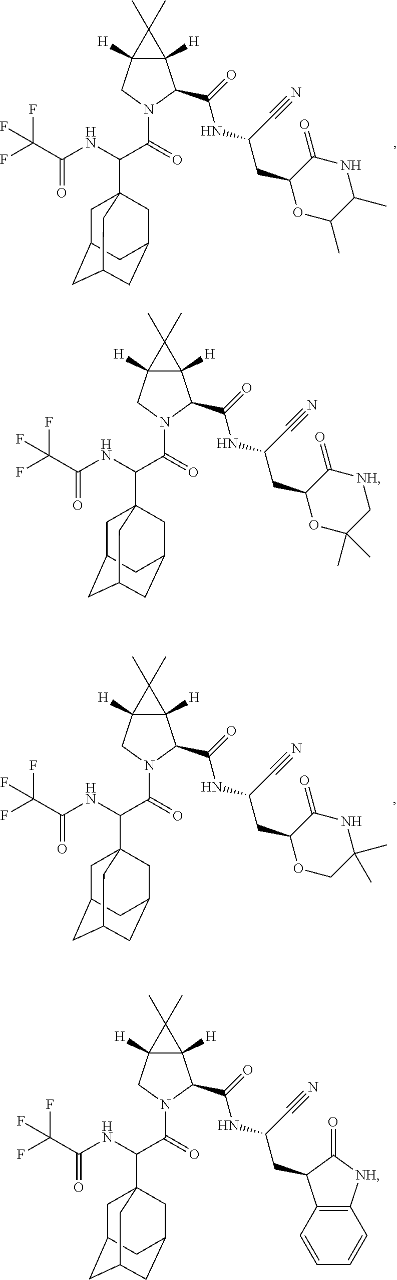

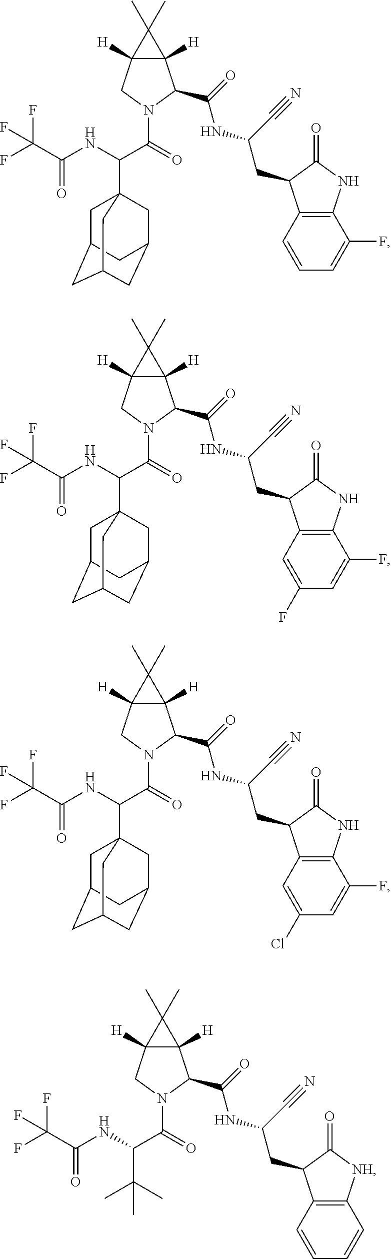

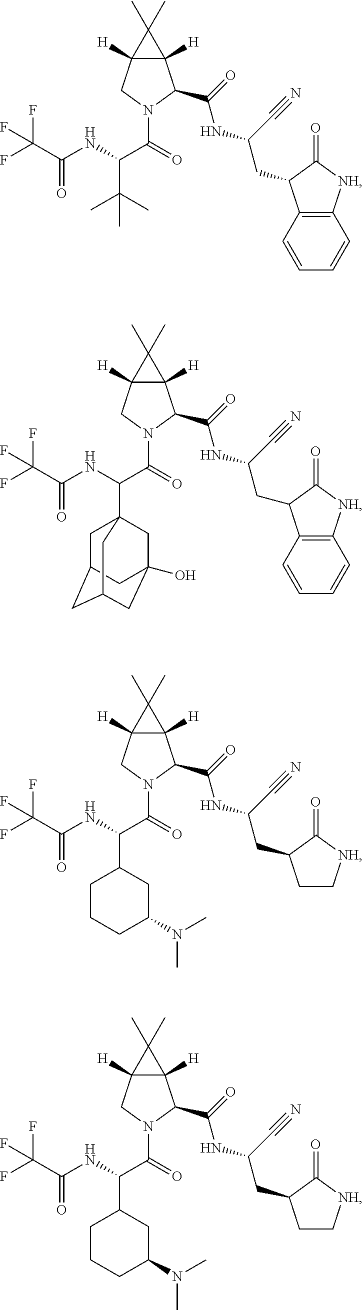 [SMILES: CC(C)(C)[C@H](NC(=O)C(F)(F)F)C(=O)N1C[C@H]2[C@@H]([C@H]1C(=O)N[C@H](C#N)C[C@@H]1C(=O)Nc3ccccc31)C2(C)C ; CC1(C)[C@@H]2[C@@H](C(=O)N[C@H](C#N)CC3C(=O)Nc4ccccc43)N(C(=O)C(NC(=O)C(F)(F)F)C34CC5CC(CC(O)(C5)C3)C4)C[C@@H]21 ; CN(C)[C@@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1 ; CN(C)[C@H]1CCCC([C@H](NC(=O)C(F)(F)F)C(=O)N2C[C@H]3[C@@H]([C@H]2C(=O)N[C@H](C#N)C[C@@H]2CCNC2=O)C3(C)C)C1]

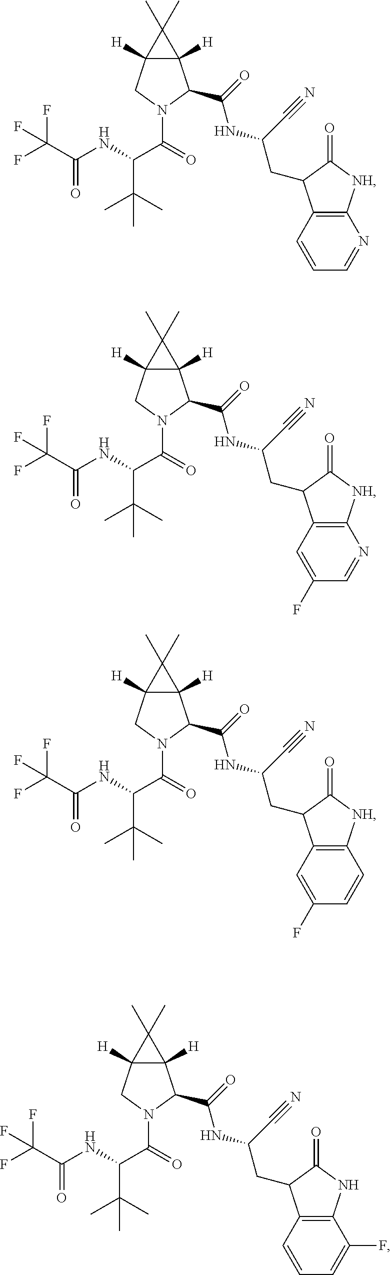 [SMILES: CC(C)(C)[C@H](NC(=O)C(F)(F)F)C(=O)N1C[C@H]2[C@@H]([C@H]1C(=O)N[C@H](C#N)CC1C(=O)Nc3ncccc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3ncc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3ccc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(F)cccc31)C2(C)C]

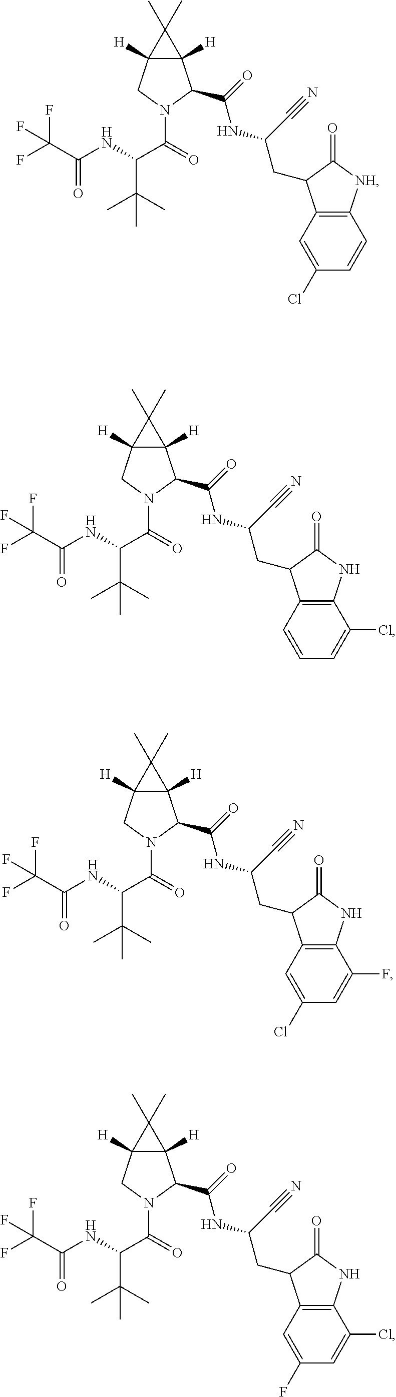 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(Cl)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cccc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(F)cc(Cl)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(F)cc31)C2(C)C]

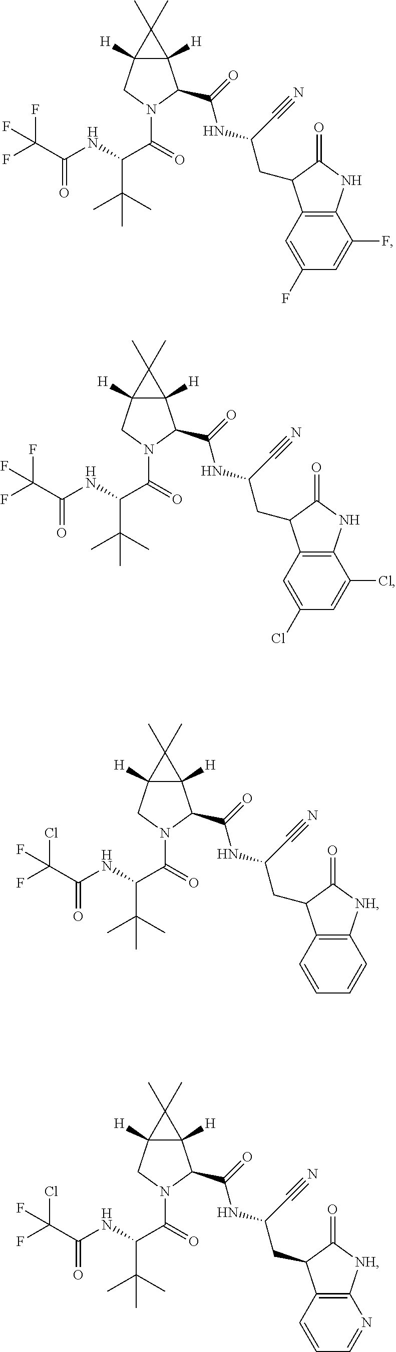 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(F)cc(F)cc31)C2(C)C ; CC(C)(C)[C@H](NC(=O)C(F)(F)F)C(=O)N1C[C@H]2[C@@H]([C@H]1C(=O)N[C@H](C#N)CC1C(=O)Nc3c(Cl)cc(Cl)cc31)C2(C)C ; CC(C)(C)[C@H](NC(=O)C(F)(F)Cl)C(=O)N1C[C@H]2[C@@H]([C@H]1C(=O)N[C@H](C#N)CC1C(=O)Nc3ccccc31)C2(C)C ; CC(C)(C)[C@H](NC(=O)C(F)(F)Cl)C(=O)N1C[C@H]2[C@@H]([C@H]1C(=O)N[C@H](C#N)C[C@H]1C(=O)Nc3ncccc31)C2(C)C]

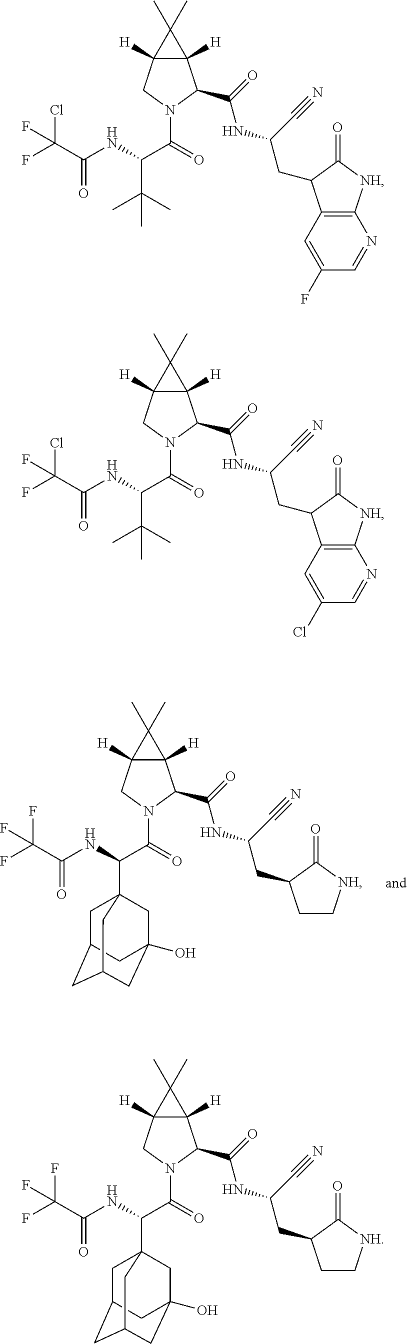 [SMILES: CC(C)(C)[C@H](NC(=O)C(F)(F)Cl)C(=O)N1C[C@H]2[C@@H]([C@H]1C(=O)N[C@H](C#N)CC1C(=O)Nc3ncc(F)cc31)C2(C)C ; CC(C)(C)[C@H](NC(=O)C(F)(F)Cl)C(=O)N1C[C@H]2[C@@H]([C@H]1C(=O)N[C@H](C#N)CC1C(=O)Nc3ncc(Cl)cc31)C2(C)C ; CC1(C)[C@@H]2[C@@H](C(=O)N[C@H](C#N)C[C@@H]3CCNC3=O)N(C(=O)[C@H](NC(=O)C(F)(F)F)C34CC5CC(CC(O)(C5)C3)C4)C[C@@H]21 ; CC1(C)[C@@H]2[C@@H](C(=O)N[C@H](C#N)C[C@@H]3CCNC3=O)N(C(=O)[C@@H](NC(=O)C(F)(F)F)C34CC5CC(CC(O)(C5)C3)C4)C[C@@H]21]

13. A pharmaceutical composition, comprising the compound or the pharmaceutically acceptable salt thereof according to claim 1 and a pharmaceutically acceptable excipient.

14. A method for preventing or treating a coronavirus 3CL protease-related disease, comprising administering to a patient Use-of-the compound or the pharmaceutically acceptable salt thereof according to claim 1.

15. A method for preventing or treating a coronavirus 3CL protease-related disease, comprising administering to a patient the pharmaceutical composition according to claim 13.
